(19) United States
(12) Patent Application Publication
(10) Pub. No.: US 2024/0156931 A1
(43) Pub. Date: May 16, 2024

(54) COLLECTION AND USES OF TISSUE RESIDENT INTERSTITIAL EXTRACELLULAR VESICLES

(71) Applicant: GEORGE MASON UNIVERSITY, Fairfax, VA (US)

(72) Inventors: Marissa Ashton Howard, Richmond, VA (US); Lance Allen Liotta, Bethesda, MD (US); Fatah Kashanchi, Potomac, MD (US); Amanda Nicole Haymond Still, Manassas, VA (US); James Lawrence Erickson, Bristow, VA (US); Alessandra Luchini Kunkel, Burke, VA (US); Rachel Carter, Vienna, VA (US); Purva Vinayak GADE, Chantilly, VA (US)

(21) Appl. No.: 18/490,053

(22) Filed: Oct. 19, 2023

Related U.S. Application Data
(60) Provisional application No. 63/417,485, filed on Oct. 19, 2022.

(51) Int. Cl. A61K 39/00; G01N 1/10; G01N 1/40; G01N 33/68

(52) U.S. Cl. CPC A61K 39/0011; G01N 1/10; G01N 1/4077; G01N 33/68; G01N 2333/70596

(57) ABSTRACT

The present invention relates to a non-destructive method comprising placing a tissue sample on a porous matrix, centrifuging the tissue sample under a negative pressure, applying a vacuum on the tissue sample, and collecting extracted extracellular vesicles from the tissue sample. The method does not cause physical rupturing of the tissue sample and maintains integrity of the tissue sample for further histopathological analysis. Moreover, the invention also provides a method of inducing an immune response of a host by immune sensitization using harvested extracellular vesicles.

### RELATED APPLICATION

This application claims priority from U.S. provisional application 63/417,485; filed on Oct. 19, 2022, entitled as, “COLLECTION AND USES THEREOF OF TISSUE RESIDENT INTERSTITIAL EXTRACELLULAR VESICLES”, which herein incorporated by reference in its entirety.

### GOVERNMENT GRANT

This invention was made with government support under grant numbers AR068436 and AI099851 awarded by the National Institutes of Health. The government has certain rights in the invention.

### FIELD OF THE INVENTION

This invention relates to a method to isolate extracellular vesicles from the interstitial space of a tissue, and possible application of the extracellular vesicles.

### BACKGROUND OF INVENTION

Extracellular vesicles (EVs) released from healthy and diseased tissue interstitial fluid (IF) into the lymphatic drainage are poorly understood aspects of EV biology. EVs circulating in the lymph constitute an information highway between tissues where the lymph is a portal of entry for tissue EVs to reach the blood. EVs may not be readily able to penetrate the blood vessels surrounding the tissue (Stacker et al., 2014) due to the vessel wall's barrier function. lymphatic vessels have high permeability to EV-sized particles (30-5000 nm), making it likely that a high proportion of EVs shed into the IF enter the draining lymph (Stacker et al., 2014) for direct delivery to the sentinel lymph node (SLN).

EVs are shed from cells embedded within the complex in vivo tissue microenvironment passively enter the interstitial space of the tissue where they are swept into the adjacent interstitial fluid (IF). The tissue IF samples from various tissue regions merge with the lymphatic drainage for downstream filtration and immune cell examination within the local sentinel lymph node (SLN) (FIG. 1a).

A portion of the EVs that enter the SLN continue on to be captured in the venous drainage and ultimately enter the general blood circulation. Despite the critically important biologic role of EVs shed into tissue IF, the molecular composition, function, and traffic of EVs from tissue into the lymphatic drainage through the IF are very poorly understood (Stacker et al., 2014).

Specifically, cancer tumor cells shed EVs over a wide range of sizes (30-5000 nm) and molecular characteristics to facilitate tumor pathogenesis, growth, immune recognition, and tumor invasion and metastasis (Lowry et al., 2015; Muller et al., 2016; Raposo and Stoorvogel, 2013; Thomas et al., 2014; Vader et al., 2014). Consequently, a high percentage of the EVs elaborated by the tumor would first encounter the innate immune system at the SLN (FIG. 1a).

Cancer EVs can be involved in anti-apoptosis, antigen presentation, immune recognition, and immune evasion (Fleming et al., 2014; Lowry et al., 2015; Pleet et al., 2018; Vlassov et al., 2012), a majority of studies have used EVs collected from cultured cells.

Extracellular vesicles (EV) are potent mediators of signaling, transferring a myriad of molecules including nucleic acids to recipient cells and promoting diverse phenotypes such as tumor progression and metastasis.

Routine histopathological disease analysis begins in a hospital when a surgeon takes a biopsy of patient tumor for example, biopsy of a suspicious mammography or suspicious polyp following colon polyp screening. The fresh tissue is then taken to the pathology lab where it is inspected, fixed, and paraffin-embedded stained and sectioned for microscope examination and analysis. In addition, special stains and tests can be performed on the tissue.

This process usually takes two to six days for a diagnostic interpretation to come back.

WO2019/236123 provides insights into the biogenesis of EVs containing high DNA cargo and the identification of one or more functions of these unique EVs, which includes the horizontal transfer of genetic material in cancer. It is demonstrated that cells A1 undergoing oxidative stress via intermittent hypoxia, mimicking a tumor's microenvironment, produced large amounts of DNA:RNA hybrids both in the cytoplasm and EVs. ChIP-DNA seq studies showed that the EV-DNA:RNA content was enriched in mitochondrial, centromeric, pericentromeric, ribosomal and circular DNA sequences.

CN107532195B discloses the invention relates to a method for separating extracellular vesicles by using an aqueous two-phase system.

US20200171084A1 discloses methods, compositions, devices, and kits for the isolation of brain-specific exosomes. Specifically, methods, compositions, devices, and kits comprising an isolated brain-specific extracellular vesicle or exosome joined to a first binding agent that is specific for tau, β-amyloid, S100 β, neuron-specific enolase, glycoprotein A2B5, CD133, NQ01, synaptophysin, neuronal nuclei, MAB1569, polysialic acid-neural cell adhesion molecule (PSA-NCAM), or neurogenic differentiation 1 (NeuroD or Beta2), or glycosylated or phosphorylated forms of these molecules, are provided.

U.S. Pat. No. 6,997,886B2 discloses enhanced interstitial fluid collection The sampling apparatus for interstitial fluid includes a pressure ring surrounding a collection needle. The pressure ring and needle are movable relative to one another for the ring to first engage a patient's skin surface prior to insertion of the needle. Single needle extraction from dermis no EVS.

U.S. Pat. No. 9,380,964B2 discloses device for interstitial fluid extraction, production process thereof and analyzing process of interstitial fluid using the device. The device for interstitial fluid extraction, having a base material formed from a synthetic resin film, a pressure sensitive adhesive layer, a hydrogel layer formed from at least one hydrophilic polymer selected from the group consisting of polyvinyl alcohol and polyvinyl pyrrolidone, and a release layer, wherein the hydrogel layer has an area of a size that the pressure sensitive adhesive layer is exposed from around the hydrogel layer, does substantially not contain a sodium ion and causes no water separation. Microneedle extraction from skin/dermis no EVs

US20190274599A1 discloses in vivo extraction of interstitial fluid using hollow microneedles. The transdermal and/or intradermal diagnostic device comprising a combined hollow microneedle interstitial fluid (IF) extraction device and a detector can monitor biomarkers in-situ. For example, electrode transducers with optimally arrayed and designed microneedles can be combined with a suitable pumping method to determine biomarker levels in human subjects under intense physical exertion to monitor metabolic stress and fatigue. Microneedles for glucose and chemical testing no EVs.

U.S. Pat. No. 8,226,558B2 discloses analyte monitoring device and methods of use. The analyte monitor includes a sensor, a sensor control unit, and a display unit. The sensor has, for example, a substrate, a recessed channel formed in the substrate, and conductive material disposed in the recessed channel to form a working electrode. The sensor control unit typically has a housing adapted for placement on skin and is adapted to receive a portion of an electrochemical sensor. The sensor control unit also includes two or more conductive contacts disposed on the housing and configured for coupling to two or more contact pads on the sensor. A transmitter is disposed in the housing and coupled to the plurality of conductive contacts for transmitting data obtained using the sensor. The display unit has a receiver for receiving data transmitted by the transmitter of the sensor control unit and a display coupled to the receiver for displaying an indication of a level of an analyte. The analyte monitor may also be part of a drug delivery system to alter the level of the analyte based on the data obtained using the sensor. Microneedles for glucose and chemical testing no EVs.

CN113234677A discloses method for extracting exosome from in-vitro tumor tissue. The invention relates to the field of biomedicine, in particular to a method for extracting exosomes from in-vitro tumor tissues. The method comprises the following steps: cutting tumor tissue, and digesting; after digestion is stopped, centrifuging and reserving supernate; centrifuging the supernatant at 4-10° C. for 10-20 min at 3000 g of 2000-one, and retaining the supernatant; centrifuging the supernatant at 4-10° C. at 10000; filtering the supernatant, collecting the filtrate, and ultrafiltering; and (4) performing ultracentrifugation on the liquid obtained after ultrafiltration for multiple times, and collecting the precipitate to obtain the product.

CN112538459A discloses method for separating exosome in liver cancer tissue. The method for isolating exosomes from liver cancer tissue, comprising: immediately soaking a fresh HCC tissue specimen after operation in PBS containing streptomycin and penicillin double antibody after the tissue specimen is separated from the body; under the aseptic operation condition, cutting the tissue blocks into tissue fragments with the size of 1-3 mm by using scissors; rinsing blood with PBS containing double antibodies, placing the blood in a DMEM/F12 culture medium, adding type I and type IV collagenase and hyaluronidase to degrade extracellular matrix, and adding DNase to digest genomic DNA released by dead cells; incubating for 30-60 minutes by using a constant-temperature shaking table; filtering the digestive juice with 70 μm filter to remove undigested tissue debris; obtaining the exosome by a gradient centrifugation method. The method of the invention can be directly separated from the liver cancer tissue specimen after the operation and can obtain the exosome which accurately reflects the characteristics of the HCC tumor.

US20210253511A1 discloses compounds and uses for the treatment and prevention of diseases and conditions associate with or aggravated by impaired mitophagy. The present invention provides compounds and methods for the treatment and prevention of diseases and conditions associate with or aggravated by impaired mitophagy.

JP2019513019A states that much work has been done to date, but the area of extracellular vesicles has not been adequately explored. The greatest therapeutic potential of such vesicles has not yet been recognized.

Previous attempts to collect tissue derived EVs have employed organ culture or the addition of tissue damaging enzymes. Unfortunately, the EV contents released post-excision into culture media may be altered by hypoxia and physiologic shock of tissue fragment survival in the ex vivo environment (Gallart-Palau et al., 2016; Jang et al., 2019; Mincheva-Nilsson et al., 2016; Teng et al., 2017; Vader et al., 2014; Vella et al., 2017), and may not accurately reflect the true resident EVs within the tumor IF at the time of ex vivo procurement

The current prior art suggests the only methods to extract EVs from an in vivo tissue sample rely on enzymatic or physical cutting/destruction of the tissue.

Further, existing research focuses almost entirely on blood samples as the clinical source for tumor-derived EVs and suffers from low yield and difficulty authenticating a tumor-derived origin. Despite early promise, the biomarker HER2, for example, was not found to be predictive of anti-HER2 therapy outcomes when measured in dilute blood EVs. Some groups have attempted to isolate EVs from dissociated tumor biopsy organ cultures. This method greatly perturbs the physiologic state of the tissue so that it does not reflect the in vivo state at the time of the biopsy and destroys the sample for histopathological analysis.

### SUMMARY OF INVENTION

An embodiment relates to a method comprising: taking a tissue sample from a human or an animal subject, placing the tissue sample onto a collection device; applying a negative pressure on the tissue sample; harvesting extracellular vesicles (EVs) ex vivo having a size in a range about 30 nm-5000 nm from a region within the tissue sample having a tissue interstitial fluid (IF) such that harvesting of the EVs retains integrity of the tissue sample for subsequent histopathological examination; and administrating a composition containing the EVs to travel to a lymph node to remodel or activate a lymph node.

In an embodiment, the EVs comprises an immune check-point inhibitor, an autophagy pathway constituent, a mitophagy pathway constituent or combinations thereof.

In an embodiment, the EVs comprise exosomes.

In an embodiment, the lymph node comprises sentinel lymph node (SLN).

In an embodiment, the EVs comprise a signal pathway phosphoprotein.

In an embodiment, the EVs comprises the mitophagy pathway constituent comprising PINK1, p62, CD81, ARIH1, and/or LC3.

In an embodiment, the EVs have a size range of about 80 nm to about 500 nm.

In an embodiment, the exosomes comprise CD81 and PINK1.

In an embodiment, the EVs harvested from the tissue sample are sensitized.

In an embodiment, the tissue sample is a tumour.

In an embodiment, irradiating the tumour of the human or an animal subject with a radiation.

In an embodiment, the radiation is configured to upregulate production of an autophagy or a mitophagy protein in the tumour.

In an embodiment, the radiation is configured to increase production of the EVs in the human or the animal subject.

In an embodiment, the method does not add tissue damaging enzymes to harvest EVs from the tissue sample.

In an embodiment, the EVs harvested from the tissue sample comprised of Exosomes (100 K EVs), endosomes (10 K EVs) and multivesicular bodies (2K EVs).

In an embodiment, the method further comprises a density gradient separation of the EVs harvested from the tissue sample.

In an embodiment, the exosomes comprise a mitophagy fission pathway constituent.

In an embodiment, the exosome comprise the mitophagy fission pathway constituent more compared to a mitophagy fusion pathway constituent or a mitochondria-associated apoptosis pathway constituent.

In an embodiment, a proteomic content of the EVs recapitulate at least 25% or more of proteins present in EVs isolated using an in vitro method as measured by mass spectrometer (MS).

In an embodiment, a proteomic analysis of the multivesicular bodies comprises an inflammatory pathway protein more than an immune cell activation pathway protein, as measured by MS.

In an embodiment, the composition comprises a cytokine.

In an embodiment, the composition the cytokine and the exosomes.

In an embodiment, administering the EVs comprising exosomes in a patient is configured to reduce a tumour progression.

In an embodiment, the mitophagy pathway constituent comprises a parkin independent mitophagy protein and/or parkin dependent mitophagy protein.

In an embodiment, the cytokine comprises CCL18/PARC or CXCL9/MIG.

In an embodiment, the tissue sample is unstained and untreated.

In an embodiment, the tissue sample is a living tissue.

In an embodiment, the method is configured not to physically rupture cells present within the tissue sample.

In an embodiment, a fluid containing harvested EVs are collected in a collection vessel containing a preservative to reduce enzymatic degradation of the fluid.

In an embodiment, method comprises purification of the EVs harvested from the tissue sample.

In an embodiment, the patient comprises the human or the animal subject from which EVs were harvested.

In an embodiment, the composition is a vaccine.

In an embodiment, the tissue sample is an ex-vivo tissue.

An embodiment relates to a composition comprising extracellular vesicles (EVs) comprising proteins that are components of mitochondria produced during cellular mitophagy of a cell, wherein the proteins are modified by exposure of the cell under a stress, wherein on administration of the composition in an animal or a human subject, it is configured to induce immune remodelling of a lymph node.

In an embodiment, a stimulant that is configured to recruit immune cells to recognize the EVs administered in the animal or the human subject.

In an embodiment, the stimulant comprises a cytokine.

In an embodiment, the cytokine comprises CCL18/PARC or CXCL9/MIG.

In an embodiment, the proteins comprise of Parkin-independent pathway.

In an embodiment, the stress is an oxidative stress or a nutritive stress or a mitochondrial DNA damage.

In an embodiment, composition comprises a carrier.

In an embodiment, the EVs comprising of 100 K EVs.

In an embodiment, the components comprise of mitochondria fragments derived from one or more stages of a mitochondrial fission.

In an embodiment, the mitochondrial fission comprises a mitochondrial peripheral fission.

In an embodiment, the composition is configured to treat a neurodegenerative, a muscular dysfunction, or a neoplastic disorder.

In an embodiment, the carrier comprises a nanoparticle carrier.

In an embodiment, the cytokine and EVs are loaded together on a carrier.

In an embodiment, the cytokine and EV are loaded onto a first carrier and a second carrier; wherein the first carrier and the second carrier are not same.

In an embodiment, the composition further contains an excipient.

In an embodiment, the composition is configured for a personalized pharmaceutical for an individual patient.

An embodiment relates to a method of treating, comprising: taking a composition a composition comprising extracellular vesicles (EVs) comprising proteins that are components of mitochondria produced during cellular mitophagy of a cell, wherein the proteins are modified by exposure of the cell under a stress, and administrating the composition in a lymph node of an animal or a human subject, wherein the method is configured to treat a disease comprising a neurodegenerative, muscular dysfunction, cardiac or neoplastic disorder.

In an embodiment, collecting EVs from an interstitial space of a tissue.

In an embodiment, the tissue comprises tumour.

In an embodiment, the composition is configured induce immune remodelling of a lymph node.

In an embodiment, the EVs containing the components of the mitochondria are configured to be taken up by a recipient cell contributing to the state of the disease.

In an embodiment, the animal or the human subject having the tumour is configured to undergo a therapy comprising a radiation therapy, a molecular targeted therapy, and/or a chemotherapy prior to biopsy of the tumour.

In an embodiment, the composition is configured to induce an abscopal effect in the animal or a human subject.

In an embodiment, the composition is configured to modulate an immune recognition of a disease either by interacting with an immunomodulatory protein or influencing inflammatory reaction of the animal or a human subject.

In an embodiment, the EVs comprises exosomes having a size in a range of 80 to 150 nm comprising PINK and CD81.

In an embodiment, the composition is configured to reduce progression of a tumour growth.

An embodiment related to a method comprising taking a tissue, collecting extracellular vesicles (EVs) ex vivo from an interstitial space of the tissue without using a tissue degrading enzyme and retaining integrity of the tissue for a histological examination of the tissue post the extraction of the EVs from it, and evaluating a mitophagy-associated proteins within the EVs collected from the tissue to determine a real-time metabolic state of the tissue.

In an embodiment, the tissue is subjected to a treatment.

In an embodiment, the treatment is treat an animal or a human subject suffering with a disease comprising a neurodegenerative, muscular dysfunction, cardiac or neoplastic disorder.

In an embodiment, the mitophagy associated proteins comprise at least one or more PTEN-induced kinase 1 (PINK1), dynamin-related protein 1 (DRP1), mitochondrial fission 1 protein (FIS1), Ariadne RBR E3 Ubiquitin Protein Ligase 1 (ARIH1), HUWE1, HECT, UBA, WWE Domain Containing E3 Ubiquitin Protein Ligase 1, Smad ubiquitin regulatory factor 1 (SMURF1), p62, sequestome 1, LC3, Microtubule-associated protein 1A/1B-light chain 3, phosphatidylethanolamine (PE), Sec22B, SEC22 Homolog B, Vesicle Trafficking Protein, Ras-Related Protein Rab-33B (Rab33B), HSP90/CDC37, 90 kDa heat shock protein, Cell Division Cycle 37, Voltage-dependent anion-selective channel 1 (VDAC), Translocase of Outer Mitochondrial Membrane 34 (TOM34) or Vacuolar protein sorting ortholog 35 (VPS-35).

An embodiment, further comprising evaluating a phosphorylated state of the cellular signaling pathway selected from the group consisting of HER-2, ERB family receptors, Tyrosine kinase receptors, G protein receptors, Immune related cell surface signaling.

An embodiment, further evaluating an autophagy-associated protein within the EVs, wherein the autophagy-associated protein comprises at least one or more Sequestosome-1 (p62), Microtubule-associated proteins 1A/1B light chain 3B (LC3b).

An embodiment, comprising isolating proteins from the EVs and characterizing the proteins.

An embodiment, comprising separating the EVs collected from the tissue according to their size.

An embodiment, comprising separating free proteins from the EVs extracted from the tissue.

An embodiment, the mitophagy-associated proteins within the EVs are not associated with the free proteins.

An embodiment, the autophagy-associated proteins within the EVs are not associated with the free proteins.

In an embodiment, a personalized pharmaceutical composition for an individual patient comprising extracellular vesicles (EVs) harvested from a tissue of the individual patient, wherein the individual patient is subjected to a therapy to treat a disease in the individual patient.

In an embodiment, the disease comprises a cancer.

In an embodiment, the therapy comprises a radiation therapy to treat the cancer.

In an embodiment, the tissue is a tumour tissue subjected to the radiation therapy.

In an embodiment, the EVs comprise exosomes.

In an embodiment, the EVs comprise particles having 30-150 nm diameter size.

In an embodiment, the EVs comprise CD81 and PINK1.

In an embodiment, the EVs in the composition after administration of the composition in the individual patient are configured to travel to a lymph node to sensitize an immune response in the individual patient.

In an embodiment, the lymph node comprises a sentinel lymph node.

In an embodiment, an administration of the composition in the individual patient is configured to reduce metastasis in the individual patient.

In an embodiment, composition comprising extracellular vesicles (EVs) harvested from a tissue, comprising proteins comprising mitophagy associated mitochondrial proteins produced during a cellular mitophagy of one or more cells of the tissue, wherein the tissue is subjected to a stress, wherein the EVs are configured to provide an indicator on a cellular metabolic state of the tissue.

In an embodiment, a stress on the living tissue comprises an oxidative stress or a nutritive stress.

In an embodiment, the tissue comprises a tumour tissue comprising a living tumour tissue.

In an embodiment, the EVs comprises particles having diameter is a range of about 30 nm-about 5000 nm

In an embodiment, the EVs comprise the particles having about 30 nm to about 150 nm diameter size.

In an embodiment, the EVs comprises a mitophagy associated mitochondrial proteins comprising at least one of PTEN-induced kinase 1 (PINK1), dynamin-related protein 1 (DRP1), mitochondrial fission 1 protein (FIS1), Ariadne RBR E3 Ubiquitin Protein Ligase 1 (ARIH1), HUWE1, HECT, UBA, WWE Domain Containing E3 Ubiquitin Protein Ligase 1, Smad ubiquitin regulatory factor 1 (SMURF1), p62, sequestome 1, LC3, Microtubule-associated protein 1A/1B-light chain 3, phosphatidylethanolamine (PE), Sec22B, SEC22 Homolog B, Vesicle Trafficking Protein, Ras-Related Protein Rab-33B (Rab33B), HSP90/CDC37, 90 kDa heat shock protein, Cell Division Cycle 37, Voltage-dependent anion-selective channel 1 (VDAC), Translocase of Outer Mitochondrial Membrane 34 (TOM34) or Vacuolar protein sorting ortholog 35 (VPS-35).

In an embodiment, the EVs containing CD81 and PINK1 are configured to provide information about a metabolic state of the tumour tissue.

In an embodiment, EVs produced from the tumour tissue comprises one or more tumour tissue specific biomarker.

In an embodiment, the one or more tumour tissue specific biomarker configured to indicate health of a specific region of the tumour tissue.

In an embodiment, the mitophagy associated mitochondrial proteins comprises mitochondrial fragments derived from one or more stages of a mitochondrial fission.

In an embodiment, the mitochondrial fission comprises a mitochondrial peripheral fission.

In an embodiment, the EVs comprise an autophagy associated proteins.

In an embodiment, the autophagy associated proteins comprising at least one of Sequestosome-1 (p62) or a Rab protein.

In an embodiment, one or more biomarkers present on the EVs determine cellular or spatial origin of the EVs.

In an embodiment, the mitophagy associated mitochondrial proteins comprises macromolecules associated with a Parkin independent pathway.

In an embodiment, the mitophagy associated mitochondrial proteins comprises macromolecules associated Parkin dependent pathway.

In an embodiment, the tissue is subjected to a therapy comprising a radiation therapy, immunotherapy, molecular targeted therapy, and/or a chemotherapy prior to biopsy of the tissue.

In an embodiment, A method of treating, comprising: taking a composition comprising extracellular vesicles (EVs) comprising proteins comprising mitophagy associated mitochondrial proteins, wherein the proteins are modified by exposure of the cell under a stress, and administrating the composition in a lymph node of an animal or a human subject, wherein the method is configured to treat a disease.

In an embodiment, the disease comprising a neurodegenerative, muscular dysfunction, cardiac or neoplastic disorder.

In an embodiment, the composition comprises an adjuvant comprising a cytokine.

In an embodiment, the cytokine comprises one or more of CCL18, CXCL9, IL-8 or MIP1-alpha.

In an embodiment, the IL-8 and the MIP1-alpha are in about 1:1 ratio.

An embodiment, comprising one or more carriers configured to load one or more components of the composition.

In an embodiment, one or more carriers comprising one or more particles comprising nanoparticles.

In an embodiment, comprising a pharmaceutically acceptable excipient.

In an embodiment, a vaccine comprising the composition prepared according an embodiment of present invention.

In an embodiment, the composition is configured to induce an abscopal effect in the animal or a human subject.

In an embodiment, the EVs are harvested from an in vivo tissue or ex vivo tissue.

In an embodiment, the EVs are produced using an in vitro method.

### BRIEF DESCRIPTION OF THE FIGURES

The patent or application file contains at least one drawing executed in color. Copies of this patent or patent application publication with color drawing(s) will be provided by the Office upon request and payment of the necessary fee.

The figures are furnished with the application to understand the invention sought to be patented. It shall not be construed as the only way to perform the invention sought to be patented.

FIG. 1A show methodology of collecting and characterizing tumour interstitial fluid EVs. Tumour cells shed EVs into the interstitial fluid (IF) of the tumour microenvironment. The IF becomes lymphatic drainage that carries the EVs to the sentinel lymph node (SLN).

FIG. 1B shows show methodology of collecting and characterizing tumour interstitial fluid EVs. IF resident EVs (green particles) are harvested from fresh, solid tumour tissue by low speed centrifugation.

FIG. 1C shows show methodology of collecting and characterizing tumour interstitial fluid EVs. molecular characterization of EVs includes isolation and purification by differential ultracentrifugation, density gradient ultracentrifugation, and immunoprecipitation with downstream analysis by western blot, mass spectrometry, electron microscopy, and fluorescent imaging of cellular molecular tags that become EV cargo.

FIG. 2A show all the major categories of IF and in vitro EVs contain the tumour cell specific eGFP marker, verifying EV tumour cell origin in vivo. eGFP-4T1 mammary carcinoma cells cultured to confluency release GFP-expressing EVs into all the common EV subtypes (2K, 10K, and 100K). The GFP-expressing EVs contain specific EV markers on their surface with the highest expression levels in the 100K fraction.

FIG. 2B shows show all the major categories of IF and in vitro EVs contain the tumour cell specific eGFP marker, verifying EV tumour cell origin in vivo. 106 GFP-4T1 cells/ml were injected into the mammary fat pad of the syngeneic BALB/c mouse model. After 2 weeks, tumours were excised and placed in an EconoSpin column lined with glass wool and gently centrifuged at 8160×g for 10 min to isolate bulk EVs from the tumour IF.

FIG. 2C show all the major categories of IF and in vitro EVs contain the tumour cell specific eGFP marker, verifying EV tumour cell origin in vivo. Bulk EVs isolated from the tissue were fractionated by ultracentrifugation at 2000×g (2K) for 45 min and 10,000×g (10K) for 45 min. Smaller diameter EVs (100K*) were isolated via nanoparticle capture technology from the resulting 10K fraction supernatant. Expression of GFP and common EV markers were Western Blotted.

FIG. 3A show high number and diversity of in situ IF EV cargo proteins compared to in vitro culture. Culture-derived EVs were analysed by subpopulation via mass spectrometry. The proteins returned were analysed via InteractiVenn software (Heberle et al., 2015). A majority of culture-derived EVs' proteins were shared among all subpopulations.

FIG. 3B shows analysis of IF proteins returned from the T1 tumour show that the 10K and 100K subpopulations share a greater number of proteins than between any other set of subpopulations. The 100K EVs contained the greatest proportion and number of unique proteins.

FIG. 3C shows comparison of culture EVs and T1 derived EVs indicate that tumour-derived EVs of the 10K and 100K populations contain greatest number of distinct, different proteins by a factor of five.

FIG. 3D shows total number of in vitro versus in vivo EV proteins derived via MS demonstrate IF EVs rich repertoire of proteomic contents.

FIG. 4 show protein pathways analysis reveal glycolytic, neurodegenerative, and pro-angiogenic pathways are the most similar comparing in vitro to in vivo. (a) Fewer proteins are shared between the culture (green) and tumour-derived (orange) EVs for the 2K population; however, the shared pathways enriched are similar to the 10K and 100K populations. (b) 10K tumour-derived EVs contain a majority of unique peptides compared to their culture counterparts. However, the 10K and 100K EVs are both enriched for peptides associated with T cell activation and Ras pathways. (c) Similar to the 10K comparison, the 100K EVs from the tumour have a majority of unique peptides. Nevertheless, all shared pathways enriched between the EVs are indicative of neurodegenerative processes, as seen by the Parkinson's and Huntington's disease pathways, and all subpopulations are enriched for pro-angiogenic factors as seen by the fibroblast growth factor signalling pathway and angiotensin-II stimulated signalling.

FIG. 5A shows IF EVs and in vitro EVs exhibit characteristics of autophagosomes and mitophagy by TEM and Western Blotting. Western Blot analysis of culture-derived EVs reveal secreted autophagy and mitophagy related structures within all EVs. The presence of LC3-I/II and p62 indicate that these EVs are autophagosomes. Moreover, PINK-1 is the central initiator of the mitophagy process additionally found in the EVs.

FIG. 5B shows Tumour-derived EVs confirm the results of the culture EVs and further indicate that the EVs released are involved in the secretory autophagy and mitophagy process.

FIG. 5C shows Transmission Electron Microscopy of culture 4T1 EV subpopulations visualizes classical autophagosome characteristics such as double-membraned vesicles with internal contents for each subpopulation. By population, the 2K EVs display a heterogenous population with internal vesicle structures, and the 100K EVs are homogenous in size and structure

FIG. 6A shows EV subpopulation localization of key autophagy, mitophagy, and checkpoint markers. Autophagosome specific p62, LC3-1/II, and checkpoint inhibitor PD-L1 are localized to the 100K CD81-enriched EVs.

FIG. 6B shows Density gradient fractions reveal co-localization of PINK1 and CD81.

FIG. 6C shows Immunoprecipitation of CD81+ EVs after density gradient ultracentrifugation were probed for Western Blot.

FIG. 6D shows Anti-CD81 immunoprecipitated EVs were enriched in PINK1.

FIG. 6E shows Five-day-old eGFP cultures were collected and differentially centrifugated to remove the 2K and 10K populations. The resulting supernatant was mixed with ExoMax solution to enrich for EVs and exclude free protein from the sample. Furthermore, to isolate the separate EV populations within the ExoMax EV concentrate, the sample was placed onto an IZON 35 nm size exclusion column. The resulting fractions were pooled into sets of 5, Nanotrapped, and analysed by Western blot.

FIG. 6F shows the proteomic content of each set of pooled fractions found two distinct groups of EV populations (fractions 6-10 and fractions 16-20). Western blot for PINK1 revealed that this marker was preferentially within the 16-20 fraction group enriched in 35-50 nm EVs and tracked with CD9 and eGFP.

FIG. 6G shows TEM images of eGFP-4T1 100K EVs contained two distinct populations of larger EVs (>100 nm, red) and smaller EVs (<100 nm, blue) by TEM.

FIG. 7A show mitophagy inducement of eGFP 4T1 EVs by CCCP correlates with PINK1 expression. GFP-4T1 cells treated with increasing levels of CCCP lead to a release of increased Parkin-independent mitophagy-containing 100K EVs (ARIH1+/PINK1+/CD81+).

FIG. 7B shows Inducing mitophagy (10 nM CCCP) within sub confluent eGFP-4T1 cells that are labelled with the mitochondrial membrane fluorescent tag MitoTracker Deep Red (MTDR) lead to an increased release of 100K EVs containing active mitochondrial molecules. MTDR fluorescence intensity was normalized to GFP fluorescence intensity by EV subpopulation.

FIG. 8A shows functional Role of EV associated Parkin-independent mitophagy-related proteins. Hypothetical depiction of autophagy and mitophagy processes within the cell and their potential relation to interstitial EVs based on the results of this study. Inside the cell, unconventional protein secretion is taking place, such as secretory autophagy and mitophagy, where organelles associated with each of these types of unconventional protein secretion can be participants in the repertoire of EVs that are passively or actively secreted into the tumour or in vivo interstitium. The legend in the upper center shows the sequence-specific proteins found in high abundance in the tumour-derived 100K EVs that are related to the Parkin-independent process of mitophagy.

FIG. 8B shows known functional location of this complex of proteins that play in the mediation of mitophagy. EV, extracellular vesicle; AP, autophagosome; MVB, multivesicular body; ILV, intraluminal vesicle; PINK1, PTEN-induced kinase 1; DRP1, dynamin-related protein 1; FIS1, mitochondrial fission 1 protein; ARIH1, Ariadne RBR E3 Ubiquitin Protein Ligase 1; HUWE1, HECT, UBA And WWE Domain Containing E3 Ubiquitin Protein Ligase 1; SMURFI, Smad ubiquitin regulatory factor 1; Ub, ubiquitin; p62, sequestome 1; LC3, Microtubule-associated protein 1A/1B-light chain 3; PE, phosphatidylethanolamine; Sec22B, SEC22 Homolog B, Vesicle Trafficking Protein; Rab33B, Ras-Related Protein Rab-33B; HSP90/CDC37, 90 kDa heat shock protein and Cell Division Cycle 37; VDAC, Voltage dependent anion-selective channel 1 (Camara et al., 2017); TOM34, Translocase Of Outer Mitochondrial Membrane 34 (Faou and Hoogenraad, 2012); TOM20, Translocase Of Outer Mitochondrial Membrane 20 VPS-35, Vacuolar protein sorting ortholog 35.

FIG. 8C show functional Role of EV associated Parkin-independent mitophagy-related proteins. Hypothetical and known functional role is depicted in FIGS. 8A and B respectively.

FIG. 9 shows tumor tissue sample both before (A) and after (B) isolation of resident IF EVs, full intact histomorphology for diagnosis, lack of perturbation for downstream clinical/research studies of the tumor tissue.

FIG. 10: shows EV isolation and purification step. EVs isolated from tumor interstitial fluid can be purified for downstream characterization.

FIG. 11: shows methodology steps according to one embodiment of this invention. It shows that initial biopsy material collected as part of the standard of care can provide a source of clinical material for EV isolation. Following collection, the biopsy material can be subjected to a gentle one step IF EV collection consisting of a two minute interstitial fluid harvest to isolate IF resident EVs, which can be purified to fractionate into subspecies, then molecularly characterized to ultimately provide biomarkers for use in clinical decision making or personalized therapy.

FIG. 12: shows RPPA analysis determines HER2 pathway activation. Specific detection of HER1 and partner phosphorylation sites indicate activation.

FIG. 13: shows that the IHC−/pHER2+ subpopulation of frozen, micro dissected tumors demonstrate full HER-signaling pathway activation. A) Pathway diagram of selected HER signaling pathway downstream proteins. B) Comparison of HER pathway activation levels between the IHC−/pHER2− cohort (N=87), IHC−/pHER2+ cohort (N=9) and IHC+/pHER2+ cohort (N=28).

FIG. 14: shows comparison of RPPA endpoint discrimination of pCR for all HER2+ patients vs. hormone receptor (HR) subsets. FIG. 13 shows the patients treated with T&P; and pCR levels defined as pathological complete response to therapy in the patients. Patients without complete response to therapy are shown in yellow; pCR patients with complete response to therapy are shown in purple.

FIG. 15: shows Anti-pEGFR(Y1068) RPPA with EVs before and after treatment. Different EV subpopulations show altered pEGFR content following treatment; all samples stained on a single slide. Sensitivity of EV RPPA: Neat solutions˜10¹⁰ EV/mL, spots size 10 nL, ˜10⁵ EVs/spot visible.

FIG. 16 shows that specific populations of mitophagy protein containing EVs are hypothesized to sensitize the immune system at the lymph node, reducing metastases.

FIG. 17 shows that separation of various size EVs using field flow fractionation (FFF) is efficient and requires small initial starting material. T-cell CEM EVs (100K) were injected into FFF-MALS using the injection loop size (100 μL) and samples separated under 60 minutes. FFF-MALS fractionate and non-destructively characterize the EV populations with little dilution. The separation mechanism of FFF is based on hydrodynamic size and a particle's natural diffusion against a gentle liquid flow.

FIG. 18 shows an Extraction device according to an embodiment of this invention.

FIG. 19 shows sensitivity analysis for N=18 mice. With expected CV=13% and sd=1.1% confidence (1−α) is between 0.95 and 0.97.

FIG. 20 shows power calculations based on pilot data suggest 10 mice/group allows for 80% power and a=0.05 to distinguish number of metastases between treated and untreated groups.

FIG. 21 shows histopathological examination process according to an embodiment of the invention.

FIG. 22 shows that Ovarian Cancer Cells undergoing cisplatin therapy release EVs which modulate in pro-survival markers and pathways. A) Ovarian Cancer tumor cells are treated with pulses of cisplatin, a common ovarian cancer therapeutic. EVs released by the cancer cells undergoing cisplatin treatment are isolated by differential ultracentrifugation and analyzed via Western Blot, mass spectrometry, and reverse phase protein array in order to determine potential pathways and markers that are indicative of cancer resistance. B) Western-blot analysis of EV subpopulations demonstrated that post-treatment EVs are upregulating cellular survival related markers such as LC3-1/II (autophagy) and upregulating immune suppression markers (PD-L1) compared to pre-treatment. Additionally, the subpopulations are present with typical EV related tetraspanin markers. C) Reverse phase protein phosphorylation analysis revealed that the ratio of upregulated cellular signaling molecules from pre to post treatment greatly increased for the EGFR (pY1068) and Bax pathways for the 2K population. For 10K Akt demonstrated an 11-fold increase. As for the 100K population, few pathways were altered after treatment with cisplatin.

FIG. 23 shows an exemplary diagram for spatial EV isolation. The methodology utilized within this exemplary diagram uses a fresh tissue sample that was sectioned with ideally a vibratome. The vibratome created thick sections of tissue which contains the EV particles necessary for diagnosis and therapeutic insights. The EV and microparticles are then isolated from the tissue section by cytocentrifuge within this exemplary method. The isolated EV particles are then ready for downstream characterization.

FIG. 24 shows an exemplary diagram for spatial EV isolation: The methodology utilized within this exemplary diagram uses a fresh tissue sample that was sectioned with ideally a vibratome. The vibratome created thick sections of tissue which contains the EV particles necessary for diagnosis and therapeutic insights. The EV and microparticles are then isolated from the tissue section by cytocentrifuge within this exemplary method. The isolated EV particles are then ready for downstream characterization by electron dispersive spectroscopy. The EVs are labelled with EV specific antibodies that are tagged with a specific metallic barcode which is quantified by the EDS scanning system.

FIG. 25 shows EV Fluorescent Particle Counting and Sizing demonstrates a 2-fold increase in EV concentration post-spin and a retention of IF EV particles within the upper chamber.

FIG. 26 shows western Blot for CD81 following PES matrix filtration.

FIG. 27 shows comparison of PES matrix with Hydrophilic bead filter matrix demonstrated the PES matrix is superior at retaining the EV portion away from the bulk IF.

FIG. 28 shows possible diagrams of a modified needle (designated as a probe) with holes at periphery.

FIG. 29A shows hollow needle with a sheath.

FIG. 29B shows retracting the sheath unblocks collection holes.

FIG. 30 shows an application of hollow needles with holes and sheath in collection

FIG. 31A shows hollow curved needle with a sheath.

FIG. 31B shows retracting the sheath in the hollow curved needle uncovers holes.

FIG. 32 shows collection of EVs using the hollow curved needle.

FIG. 33 shows a hollow needle with prongs.

FIG. 34A shows a device for collecting interstitial fluid according to an embodiment of the invention.

FIG. 34B shows a process of separation of molecules and EVS in the device according to an embodiment of the invention.

FIG. 35 shows interstitial extracellular vesicles reflect the treatment of tumor tissue prior to EV sampling. Repertoire of individual EV-associated molecules found in the interstitial fluid after treatment with immunotherapy. A total of 224 molecules were elevated in the treated tumors with a statistically significant level (pink box). Treated tumors (left) and untreated tumors (right).

FIG. S1 shows tumour histopathology remains intact following IF isolation. Tumour tissue samples both before (Pre-IF Spin) and after (Post-IF Spin) isolation of resident IF EVs retain full intact histomorphology for downstream complete pathological diagnosis or analysis at 100× magnification.

FIG. S2 shows Tumor-associated markers predominate IF proteomic content. To determine the relative amount of non-tumour specific cellular markers contained within the 100K IF sample, we identified key breast epithelial and immune markers compared to known breast tumour-associated markers found within different eGFP-4T1 100K tumour samples (N=4). Breast tumour-associated markers such as alpha-smooth muscle actin (a-SMA) and epidermal growth factor receptor (EGFR) and its substrates were in excess compared to breast tissue epithelial makers such as Keratin 5, 8, and 18. Furthermore, evidence of circulating neutrophils (CD177) and T-cell (CD5) were present in two out of four 100K IF tumours.

FIG. S3A show normalization to total protein abundance does not significantly change the dynamic range of identified peptides within culture-derived or IF-derived EVs. Relative protein abundance of T1 tumour IF EV subpopulations to culture-derived EV subpopulations show a slightly higher abundances of protein in the IF EVs.

FIG. S3B shows Normalized protein abundance of T1 IF EVs subpopulations to culture-derived EV subpopulations remove the difference between culture versus IF EVs.

FIG. S3C shows Relative protein abundance of N=3 tumours by EV subpopulation indicate protein abundance by EV subpopulation is similar.

FIG. S3D shows Normalization of protein abundances demonstrate a reliable method to compare proteomic abundances between samples.

FIG. S4A shows Lymph-node priming with EV subpopulations demonstrates an induction of tumour growth and metastasis for 2K EVs compared to a decrease in the 100K subpopulation. Schematic representation of open mesh work hydrogel nanoparticles preloaded with 1 ug/ml of cytokines (IL-8 and MIP1-alpha) in a 1:1 ratio with nanoparticles pre-loaded with 4T1 EV subpopulations (2K, 10K, and 100K).

FIG. S4B The mixture of CK/NPs and EV/NPs were subcutaneously injected into the footpad of BALB/c immunocompetent mice (N=5 per EV population). After 2 days, the mice were subjected to 4T1 cancer cell challenge.

FIG. S4C shows Histological view (4×) of the distant metastases grown in the lungs. Compared to control, the 2K EVs contributed to larger distant metastases, while the 100K was primarily associated with fewer and smaller distant metastases.

FIG. S4D shows Metastases of the lungs were counted for each population with the 2K group having a significantly higher average, whereas the 100K EVs returned fewer than control, indicative of potential immune cell priming.

FIG. S4E shows Volumetric analysis of the tumour growth further demonstrates the large induction of tumour growth for the 2K EV population.

FIG. S4F shows Repeat experiment to determine the effects of eGFP 4T1 EVs with and without cytokine injected into the orthotopic region of the mammary fat pad.

FIG. S4G shows eGFP 4T1 EVs were normalized by particles/ml and injected with and without cytokine. After 2 days post-treatment, the animals were challenged with 106 4T1 cells/mouse.

FIG. S4H shows Tumour length at day 14 post-tumour challenge indicates that the addition of cytokines to the 100K EVs leads to a reduced tumour growth rate as compared to control (no treatment).

FIG. S5A shows EV subpopulations demonstrate varied pro-tumour mechanisms. The effect of 4T1 EV subpopulations on tubular formation in vitro. EV-treated and untreated cells received a 1:2 dilution of fully supplemented medium along with the indicated treatment. EV treatments were approximately 1:2000 (recipient cell to EV ratio) and untreated cells received PBS. Representative phase contrast and fluorescent images show the formation of tubules on day 3 and day 6 of the assay.

FIG. S5B shows a Wimasis WimTube analysis was performed to quantify the (b) Total tube length.

FIG. S5C shows Total branching points. This analysis showed that the 2K population further demonstrated phenotypical characteristics of vascular growth.

FIG. S5D shows Culture-derived 2K EVs contained vascular endothelial growth factor (VEGF), a common angiogenesis marker.

FIG. S6A shows EV uptake assay. EVs were fluorescently labelled with BODIPY 493/503 and added to confluent cultures of MSCs at an approximate ratio of 1:2000 (recipient cell to EV ratio). Cells were incubated for a period of 1 to 6 days. Representative phase contrast and fluorescent images for Day 1 shows the relative morphology and EV uptake over Lime.

FIG. S6B shows EVs were fluorescently labelled with BODIPY 493/503 and added to confluent cultures of MSCs at an approximate ratio of 1:2000 (recipient cell to EV ratio). Cells were incubated for a period of 1 to 6 days. Representative phase contrast and fluorescent images for Day 2 shows the relative morphology and EV uptake over time.

FIG. S6C shows EVs were fluorescently labelled with BODIPY 493/503 and added to confluent cultures of MSCs at an approximate ratio of 1:2000 (recipient cell to EV ratio). Cells were incubated for a period of 6 days. Representative phase contrast and fluorescent images for Day 6 shows the relative morphology and EV uptake over time.

FIG. S7 shows TEM Imaging reveals heterogeneity between EV subpopulations and homogenous structures for 100K EVs. Scaled images of TEM images further demonstrate the amphisome-like qualities of the 2K and the uniformity of vesicle for the 100K EVs. Additionally, all structures have double membranes indicative of autophagosomes.

FIG. S8 shows GFP expression is reproducible in GFP-4T1 tumours harvested both from the same mouse, as well as from different mice with CV %<10% for the 100K population. GFP expression was compared between two tumours from two different mice (Trials 1 and 2) as well as two tumours from the same mouse (Trial 3). GFP was quantified via densitometry from Western Blot using ImageJ; densities were normalized to actin density per lane. All samples per trial were run on the same blot. CV % is calculated as standard deviation divided by average value, multiplied by 100.

FIG. S9 shows PINK1+IF EVs contain primarily unfragmented PINK1 that is susceptible to trypsin proteolysis. (a) IF EVs (10 μg) were probed for PINK1 and demonstrated primarily unfragmented PINK1 in the 100K EVs, although there was variation in fragmentation pattern between tumour samples. (b) 2K and 10K depleted tumour IF samples (10 μg) were probed for PINK1 and TOM20. The results revealed that tumour IF contains various fragments of PINK1 (f-PINK1) which may correspond to the cellular pressures and processing of PINK1 for different tumour samples. TOM20, a co-localized mitochondrial marker required for mitochondrial fission, corresponds with the relative amounts of PINK1 present. (c) T4 and T6 samples were further treated with high (3.5 pig) and low (0.85 pig) concentrations of trypsin to identify the localization of PINK1 on or within the IF EVs. Trypsinization revealed a heterogeneous sensitivity of full length PINK1 and its fragments to proteolysis. CD81 is shown as a known extracellular membrane control. Abbreviation: f-PINK1 (fragmented PINK1).

FIG. S10 shows a population of PINK1 EVs are not associated with free protein. (a) comparison of IZON q35 column size exclusion EV fractionation with and without ExoMax of 100K IF eGFP 4T1 EVs mixed with 10% FBS (EV-free) shows EV associated markers (Gly-CD81, CD81, and Alix) are found in fractions 6-15. PINK1 is found in fractions 11-20. (b) Silver stain quantification of the gels found that ExoMax EV precipitation successfully excludes free protein found in higher IZON fractions (31-50). (c) Within the 11-15 fraction (PINK1+), a population of EVs were identified by SEM (size bars: left (1 mm), middle (100 nm), and right (100 nm).

### DETAILED DESCRIPTION

### Definitions and General Techniques

For simplicity and clarity of illustration, the drawing figures illustrate the general manner of construction, and descriptions and details of well-known features and techniques may be omitted to avoid unnecessarily obscuring the present disclosure. Additionally, elements in the drawing figures are not necessarily drawn to scale. For example, the dimensions of some of the elements in the figures may be exaggerated relative to other elements to help improve understanding of embodiments of the present disclosure. The same reference numerals in different figures denote the same elements.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Furthermore, the terms “include,” and “have,” and any variations thereof, are intended to cover a non-exclusive inclusion, such that a process, method, system, article, device, or apparatus that comprises a list of elements is not necessarily limited to those elements, but may include other elements not expressly listed or inherent to such process, method, system, article, device, or apparatus.

The terms “left,” “right,” “front,” “back,” “top,” “bottom,” “over,” “under,” and the like in the description and in the claims, if any, are used for descriptive purposes and not necessarily for describing permanent relative positions. It is to be understood that the terms so used are interchangeable under appropriate circumstances such that the embodiments of the apparatus, methods, and/or articles of manufacture described herein are, for example, capable of operation in other orientations than those illustrated or otherwise described herein.

No element, act, or instruction used herein should be construed as critical or essential unless explicitly described as such. Also, as used herein, the articles “a” and “an” are intended to include items, and may be used interchangeably with “one or more.” Furthermore, as used herein, the term “set” is intended to include items (e.g., related items, unrelated items, a combination of related items, and unrelated items, etc.), and may be used interchangeably with “one or more.” Where only one item is intended, the term “one” or similar language is used. Also, as used herein, the terms “has,” “have,” “having,” or the like are intended to be open-ended terms. Further, the phrase “based on” is intended to mean “based, at least in part, on” unless explicitly stated otherwise.

The present invention may be embodied in other specific forms without departing from its spirit or characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

As defined herein, “approximately” can, in some embodiments, mean within plus or minus ten percent of the stated value. In other embodiments, “approximately” can mean within plus or minus five percent of the stated value. In further embodiments, “approximately” can mean within plus or minus three percent of the stated value. In yet other embodiments, “approximately” can mean within plus or minus one percent of the stated value.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, health monitoring described herein are those well-known and commonly used in the art.

The methods and techniques of the present invention are generally performed according to conventional methods well known in the art and as described in various general and more specific references that are cited and discussed throughout the present specification unless otherwise indicated. The nomenclatures used in connection with, and the procedures and techniques of embodiments herein, and other related fields described herein are those well-known and commonly used in the art.

The recitations of numerical ranges by endpoints include all numbers subsumed within that range (e.g., 1 to 5 includes 1, 1.5, 2, 2.75, 3, 3.80, 4, 5, etc.). Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present specification. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the specification are approximations, the numerical values set forth in the specific examples are reported as precisely as possible. All numerical values, however, inherently contain a range necessarily resulting from the standard deviation found in their respective testing measurements.

The present invention is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

It should be noted herein that any feature or component described in association with a specific embodiment may be used and implemented with any other embodiment unless clearly indicated otherwise.

The following terms and phrases, unless otherwise indicated, shall be understood to have the following meanings.

The term “cell” herein is defined in the same way with the broadest meaning used in the art. It is structural unit of a tissue of a multicellular organism, surrounded by a membrane structure that isolates it from the outside world. Cell culture is the process by which cells are grown under controlled conditions, generally outside their natural environment.

As used herein, the term “therapeutic agent” means an agent having therapeutic effect, including, but not limited to, chemotherapeutic agents. The term also includes vectors for gene therapy, antisense nucleic acid constructs and transcription factor attractants.

The term “therapeutically effective amount” or “therapeutically effective dose” or similar relates to an amount of a compound (including its crystal form or salt form) that is therapeutically effective to treat a disorder in an individual. The amount can be administered in a single dose or multiple doses.

The term, “carrier” or “carrier particles” refers to a delivery system associated with a therapeutic agent (e.g., the therapeutic agent is entangled, embedded, incorporated, encapsulated, bound to the surface, or otherwise associated with the carrier particle) or any other agent that may carried from one location to another. In an embodiment, the carrier particles exhibit fluorescent activity or a measurable signal when exposed to light or another external stimulus, which is useful for diagnostics. imaging and sensoring. The composition and size of the carrier particles is selected by the practitioner depending on the desired method of administration, in accordance with the therapeutic agent to be delivered. the carrier particles are microparticles (about 1-1000 μm) or nanoparticles (about 1-1000 nm). In another preferred embodiment, the carrier particles have an average diameter less than about 100 μm, about 75 prn, about 60 μm, about 50 μm about 40 μm, about 25 μm, about 20 μm, about 15 μm, about 10 μm, about 5 μm, about 2.5 μm, about 1000 nm (1 μm), 500 nm, 300 nm, 200 nm, 150 nm, 100 nm, 75 nm, 50 nm, 30 nm, 20 nm, 15 nm, 10 nm, 5 nm, or 1 nm.

The term, “nanoparticles” as used herein is called a support structure, biocompatible and sufficiently resistant to chemical and/or physical destruction by the environment of use. Nanoparticles can be solid colloidal particles having a size of 1 to 1000 nm. A nanoparticle can be any diameter less than or equal to 1000 nm, inclusive 5, 10, 15, 20, 25, 30, 50, 100, 500 and 750 nm.

The term “interstitial fluid (IF)” refers to a body fluid between blood vessels and cells, containing nutrients from capillaries by diffusion and holding waste products discharged out by cells due to metabolism. Interstitial fluid is also defined as a fluid between cells. Interstitial fluid contains a water solvent containing sugars, salts, fatty acids, amino acids, coenzymes, hormones, neurotransmitters, white blood cells, cell waste-products and more. The interstitial fluid can be collected from places such as without any limitation, CNS (brain or CSF), subcutaneously (skin), intraperitoneal, a tumor tissue or tissue subtype.

The term “extracellular vesicle (EV)” as used herein is a broad term to describe secreted vesicles secreted by cells in an extracellular space. The vesicles could be either membranous or non-membranous. Vesicles are usually lipid vesicles. EVs could be but not limited to exosomes, microvesicles, microparticles, ectosomes, matrix vesicles, calcifying vesicles, prostasomes, oncosomes, retrovirus-like particles, bacterial extracellular vesicles, intraluminal vesicles and apoptotic bodies. EVs may carry markers of cells of origin. They may have specialized functions in physiological processes, from coagulation and intercellular signalling to waste management. EVs can be derived from almost all mammalian cells, including healthy cells, stem cells, and diseased cells, such as cancer cells. EVs can exist in almost all body fluids including blood plasma, saliva, urine, bile, synovial fluid, semen and breast milk. EVs generally have a diameter in the range of 10 nm to 5000 nm. In an embodiment, EVs have a size range between 30 nm to 5000 nm. In an embodiment, EVs are at least 50 nm, 100 nm, 500 nm, 1000 nm, 1500 nm, 3000 nm or more.

The term “microvesicle” as employed herein refers to vesicles released from the cell by outward budding of the plasma membrane. The microvesicles generally have a diameter in the range 100 nm to 5000 nm. In some embodiments, the microvesicles have an upper size limit not being more than 1000 nm (i.e., 1.0 micrometer, micron, or μm), or alternatively, not more than about 1500 nm, about 2000 nm or about 2500 nm.

The term “endosomes” refer to a collection of intracellular sorting organelles in eukaryotic cells. They are part of endocytic membrane transport pathway originating from the trans Golgi network. There are three different types of endosomes: early endosomes, late endosomes, and recycling endosomes. Late endosomes are also called multivesicular endosomes or multivesicular bodies (MVBs).

The term “exosomes” are produced inside multivesicular bodies and are released after fusion of the multivesicular body with the cytomembrane. Exosomes have several properties which make them ideal for delivering material into cells, which includes their small size (e.g. able to cross the blood brain barrier), natural ability to fuse with the plasma membrane of cells to deliver their contents, stable internal environment and their ability to deliver functional molecules to the recipient cell which include: nucleic acids (DNA, mRNA and miRNA), lipids and proteins. Generally, exosomes have a diameter in the range 30 nm to 100 nm. As used herein, it is not intended that an exosome of the invention be limited by any particular size or size range.

The term “apoptotic bodies” as employed herein refer to vesicles that are shed into the extracellular environment by apoptotic cells. Apoptotic bodies may not be involved in intracellular communication. Generally, the diameter of apoptotic bodies is in the range 800 nm to 5000 nm.

The term “subject” could be a vertebrate, a mammal, or avian (e.g., bird), or other organism. Examples of subjects include, but are not limited to, a mammal such as a rodent, mouse, rat, rabbit, guinea pig, ungulate, horse, sheep, pig, goat, cow, cat, dog, primate (i.e. human or non-human primate). Terms such as individual/patient/subject are interchangeably used throughout the specification.

The term “sample” refers to clinical samples obtained from a patient. Sample could be but are not limited to, saliva, mucus, sputum (processed or unprocessed), bronchial alveolar lavage (BAL), bronchial wash (BW), blood, bodily fluids, cerebrospinal fluid (CSF), urine, plasma, serum, or tissue (e.g., biopsy material) or biological fluid, or the like.

In an embodiment, if the sample is a biopsy material, then it could be referred as a “tissue sample”.

The term “tumour tissue” refers to a cancerous tissue. The cancerous tissue could be encompassing a cancer such as but not limited to breast cancer, glioma, large intestinal cancer, lung cancer, small cell lung cancer, stomach cancer, liver cancer, blood cancer, bone cancer, pancreatic cancer, skin cancer, head or neck cancer, cutaneous or intraocular melanoma, uterine sarcoma, ovarian cancer, rectal or colorectal cancer, anal cancer, colon cancer, gastrointestinal stromal tumors (GIST), fallopian tube carcinoma, endometrial carcinoma, cervical cancer, vulval cancer, squamous cell carcinoma, vaginal carcinoma, Hodgkin's disease, non-Hodgkin's lymphoma, esophageal cancer, small intestine cancer, endocrine cancer, thyroid cancer, parathyroid cancer, adrenal cancer, soft tissue tumor, urethral cancer, penile cancer, prostate cancer, chronic or acute leukemia, lymphocytic lymphoma, bladder cancer, kidney cancer, ureter cancer, renal cell carcinoma, renal pelvic carcinoma, CNS tumor, astrocytoma, glioblastoma multiforme, oligodendroglioma, primary CNS lymphoma, bone marrow tumor, brain stem nerve gliomas, pituitary adenoma, uveal melanoma, testicular cancer, oral cancer, pharyngeal cancer, pediatric neoplasms, leukemia, neuroblastoma, retinoblastoma, pediatric glioma, medulloblastoma, Wilms tumor, osteosarcoma, teratoma, rhabdomyoblastoma and sarcoma.

In an embodiment, tumour tissue could be from the core region of the cancer. In an embodiment, the tumour tissue could be from the peripheral region of the cancer. ‘Tumour’ is also spelled as ‘tumor’.

As used herein the term “metastasis count” refers to counts of spread of cancer to other places, positions, or areas in the body. In an embodiment, the method of the present invention reduces metastasis count.

The term “non-destructive method” refers to a method which does not cause physical rupturing of the tissue. In aspect of present invention, in non-destructive method, histo-morphology of the tissue is retained after post extraction procedure of EVs from the tissue. Thus, non-destructive method allows genetic and molecular analysis of the tissue after the extraction of EVs also from the tissue. In an embodiment, non-destructive method leads to no to minimal destruction of tissue as understood by a person skilled in the art, such as less than 5% destruction of the tissue.

The term “physical rupturing” refers to complete or partial breakage of cells of a tissue sample by application of an external object such as an injection. In an embodiment, there is no breakage of cells in the tissue sample. In an embodiment, the method is completely free of breakage of cells of the tissue sample. In an embodiment, the method may rupture less than 25%, less than 20%, less than 10%, less than 5%, less than 1% of the cells present within the tissue sample.

The term “integrity” refers to the state of maintaining an internal molecular consistency of the tissue sample at pre and post extraction procedure. Maintaining integrity of the tissue sample is to maintain the original state of the tissue sample as it was present before application of the method as defined in this specification.

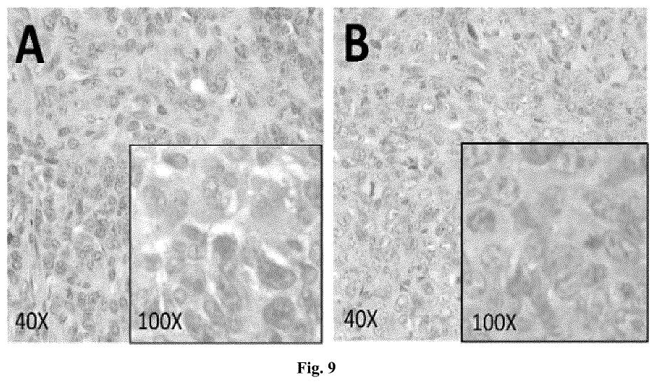

Integrity refers to histologically intact (as shown in FIG. 9), with no perturbation in nuclear fine chromatic structure, cell morphology or size. The size and shape of the carcinoma cell body is unaltered. Furthermore, the fine nuclear chromatin, nuclear membrane and nucleoli, as well as all other morphologic elements are identical for post IF harvesting tumor tissue as compared to freshly excised intact tumors.

In an embodiment, integrity refers to maintaining refers 100% of internal molecular consistency of the tissue sample. In an embodiment, integrity of the tissue is maintained about 90% or more of the original state. In an embodiment, integrity of the tissue is maintained about 80% or more of the original state. In an embodiment, integrity of the tissue is maintained about 70% or more of the original state. In an embodiment, integrity of the tissue is maintained about 60% or more of the original state.

The term “histopathological analysis” refers to the examination of a tissue sample by a pathologist. It could be for locating, analyzing and classifying molecules, cells and studying structural integrity of the tissue.

The term “cellular morphology” or “cell morphology” or “cytomorphology” refers to study of the morphological features of the cell. The cell could be an individual cell, cell within a group of cells, or cell within a tissue. Morphological features of the cells could be such as but not limited to size, shape, structure, form, color, texture, composition within the cell, molecules within the cell, organelles within the cell, etc.

The term “unstained” refers to tissue sample that is not stained or attached using various stains and tags to enhance contrast of the tissue components and thereby improve visibility of the tissue. The terms “stain” and “staining” are broad terms and can include without limitation staining with a dye, a stain, immunohistochemical staining, aptamer staining, tagging, chemical staining, antibody staining, or any other alteration done to improve the visibility of a tissue sample.

The term ‘untreated” refers to a tissue sample that has not being subjected to external enzymes such as but not limited to tissue extraction enzymes or a chemical. Chemicals could be such as but not limited to fixing agents that may alter the characteristics of the tissue, such as but not limited to improve anchorage of the tissue, visibility of components of the tissue. The treatment of the tissue may be used to ease extractions of components from the tissue, etc.

The term “living tissue” refers to tissues that are product of the nature. The living tissues may have ability to grow by changing their mass and remodel by changing their internal structure in response to diverse stimuli. Living tissue could be an in vivo tissue or ex vivo tissue. In vivo tissue refers to tissue present at its place of origin. Ex vivo tissue refers to Ex vivo (Latin: “out of the living”) literally means that which takes place outside an organism. In science, ex vivo refers to experimentation or measurements done in or on tissue from an organism in an external environment with minimal alteration of natural conditions.

The term, “in vitro EVs” refers to cell culture-derived EVs.

The term, “ex vivo EVs” refers to EVs extracted from a living tissue.

The term “cytokine” is defined in the broadest meaning used in the art and refers to a physiologically active substance produced from a cell, that may act on the same or different cells. Cytokines are generally proteins or polypeptides, and have a suppressive action on the immune response, regulation of the endocrine system, regulation of the nervous system, antitumor action, antiviral action, regulation of cell proliferation, regulation of cell differentiation, etc. As used herein, cytokines can be in protein or nucleic acid form or other forms, but at the point of action, cytokines usually mean protein forms. In general cytokines are involved in cell signalling and immune response. Cytokines include chemokines, interferons, interleukins, lymphokines, and tumour necrosis factors, but generally not hormones or growth factors (despite some overlap in the terminology).

The term “disease” refers to an abnormal condition that negatively affects the structure or function of all or part of an organism, and that is not immediately due to any external injury. In an embodiment, the disease is a cancer. In some embodiments, the disease may be a localized or non-localized. In an embodiment, disease includes for example but not limited to, a muscular dysfunction, a neoplastic disorder, a dermal disease such as atopy, a respiratory disease, such as rhinitis, sinusitis, nasopharyngeal cancer, bronchitis, asthma, chronic obstructive pulmonary disease, bronchiectasis, pneumonia, and lung cancer, a digestive disease such as stomatitis, oral cavity cancer, esophagitis, esophageal cancer, gastritis, stomach cancer, inflammatory bowel disease, and colorectal cancer, and a genital disease such as vaginitis, cervicitis, and uterine cervical cancer. In some other embodiments, the disease may be a systemic disease including, but not limited to, a vascular disease such as sepsis, thrombosis/embolism, arteriosclerosis, stroke, acute coronary syndrome, and ischemic vascular disease, a metabolic disease such as diabetes and obesity, a pulmonary disease such as emphysema, and acute respiratory distress syndrome, a bone disease such as arthritis and osteoporosis, and a cranial nerve disease such as dementia, neurodegenerative diseases, and depression. In an embodiment, the disease is a blockade of a communication process between a first cell and a second cell, wherein the first cell elaborates a first extracellular vesicle containing a component of a mitophagy pathway and/or autophagy pathway and the first extracellular vesicle is taken up by the second cell. In another embodiment, the component belongs to a fission pathway of mitophagy. In yet another embodiment, the method is a therapeutic method to treat a disease.

The term, “autophagy” describes “self-eating,” is a homeostatic response to degrade and recycle cellular material for energy. Autophagy leads to secretion of specific autophagic proteins. These proteins may be secretory or non-secretory. The autophagy includes secretory autophagy that utilizes similar autophagy machinery for unconventional protein secretion. In an embodiment, the present invention relates to molecular mediators specific to secretory autophagy such as (SEC22b and RAB33b) but not limited to them.

The term, “mitophagy” describes a selective form of autophagy specifically for the removal of damaged or aged mitochondria. There are two major pathways which can induce the mitophagic process: Parkin-dependent mitophagy and Parkin-independent mitophagy. In an embodiment, mitophagy includes both parkin-dependent and parkin-independent process. In an embodiment, mitophagy includes only parkin-independent process.

Mitophagy is preceded by mitochondrial fission factor 1 (FIS1) that trigger peripheral fission to detach end segments of mitochondria for autophagic processing. Mitochondrial function and energy metabolism are crucial components of normal and diseased tissue cell function. In an embodiment, mitochondrial proteins are present within EVs.

The term “porous matrix” refers to a matrix with voids or pores that allow passage of fluids. The fluid could contain solids dissolved in it. The pore size of the porous matrix is small enough to allow a tissue sample to rest on it and does not allow the tissue to pass through the pores. In an embodiment, the matrix or frame of the porous matrix should be strong enough to bear the vacuum pressure or negative pressure. In an embodiment, the pore size can be but not limited to 30 to 10,000 nm. In an embodiment, the pore size is less than 8000 nm. In an embodiment, the pore size is less than 7000 nm, less than 6000 nm, less than 5000 nm, less than 2000 nm, 1000 nm, 500 nm or less. In an embodiment, the porous matrix comprises glass wool. In some embodiments, term porous matrix and matrix are interchangeably used.

The term “negative pressure” or “reduced pressure” refers to a pressure created using a vacuum pump or like to decrease the pressure within a device compared to ambient pressure outside the device. Due to decrease in pressure in the device, extracellular vesicles can rush from the tissue sample, in a reservoir or device. In an embodiment, the negative pressure is equivalent to an interstitial pressure. In another embodiment, negative pressure is pulsed or continuous. In an embodiment, pulse pressure is when a decrease in pressure is in strokes and with a time difference between two consecutive strokes.

The term “immune sensitization” refers to a process to elicit immune response in the subject. In an embodiment, ‘immune sensitization’ could be achieved by an adoptive transfer of cells such as immune cells to a patient.

The term “abscopal effect” refers to treatment such as a radiation treatment or a local therapy that shrinks the targeted tumor and leads to the shrinkage of untreated tumors elsewhere in the body. It is a treatment for metastatic cancer whereby shrinkage of untreated tumors occurs concurrently with shrinkage of tumors within the scope of the localized treatment.

The term “biomarker” or “biological marker” broadly refer to any characteristics that are objectively measured and evaluated as indicators of normal biological processes, pathogenic processes, or pharmacologic responses to therapeutic intervention. Unless otherwise noted, the term biomarker as used herein specifically refers to biomarkers that have biophysical properties, which allow their measurements in biological samples (e.g., plasma, serum, cerebrospinal fluid, bronchoalveolar lavage, biopsy). Unless otherwise noted, the term biomarker is used interchangeably with “molecule biomarker” or “molecular markers.” Examples of biomarkers include nucleic acid biomarkers (e.g., oligonucleotides or polynucleotides), peptides or protein biomarkers, lipids, and lipopolysaccharide markers.

In an embodiment, biomarker comprising a phosphorylated signal pathway protein, a mitophagy biomarker, a check-point inhibitor or an autophagy biomarker. In another embodiment, phosphorylated signal pathway protein comprises HER-2, ERB family receptors, tyrosine kinase receptors, G protein receptors.

The term “collection vessel” refers to a vessel or a chamber or reservoir or like to collect a desired substance. The collection vessel could be either component of a device or could be attached separately to a device. The collection vessel could be of varied shapes and sizes. Further the collection vessel could be made up of material such as but not limited to plastic, steel, etc. The material of the collection vessel is such that the substance to be collected in the vessel does not react with it. In case the material of the collection vessel is reactive, then it could have a coating or a layer or matrix over the material such that the substance does not react on storage inside the collection vessel. In an embodiment, the collection vessel collects EVs. The collection vessel or chamber could have other components in it such as a preservative.

The term “preservative” refers to a chemical that is added into products to prolong shelf-life of the product either by preventing decomposition of the products by microbial growth or by controlling undesirable chemical changes, The products could be such as chemical compounds such as a pharmaceutical drug, biological products such as an antimicrobial agent, etc. In an embodiment, the collection vessel comprises a preservative.

The term “vacutainer tube” refers to a tube made up of a material such as glass or plastic, and vacuum sealed with a stopper. The tube facilitates drawing of a predetermined volume of liquid. In an embodiment, the collection vessel and the chamber could be connected via a vacutainer tube.

The term “radiation therapy” also referred as “XRT”, generally means the use of ionizing radiation to kill cells such as cancer cells as a part of anticancer treatment. X-rays, gamma rays, or charged particles (eg, protons or electrons) are used to generate ionizing radiation. Radiation therapy may be delivered by equipment placed outside the patient's body (extracorporeal radiation therapy) or from a source located inside the patient (internal radiation therapy or brachytherapy), or intravenously or orally. It may be delivered by the delivered systemic radioisotope (systemic radioisotope therapy). Radiation therapy may be planned and administered with imaging-based techniques such as computer tomography (CT), magnetic resonance imaging (MRI) to accurately determine the dose and location of radiation to be administered. In various embodiments, the radiotherapy includes whole body radiotherapy, conventional extracorporeal radiotherapy, stereotactic radiotherapy, stereotactic radiotherapy, three-dimensional conformal radiotherapy, intensity-modulated radiotherapy, image-guided radiotherapy, tomotherapy, choline. It is selected from the group consisting of source therapy and systemic radiation therapy. In certain embodiments, radiation therapy is curative, adjunctive, or palliative, depending on its intent. In certain embodiments, the term “radiation therapy” refers to subdivided radiation therapy. Small-split radiation therapy refers to radiation therapy in which the radiation dose is included in two or more splits. U.S. Ser. No. 11/077,141B2 and U.S. Ser. No. 10/421,971B2 describing radiation therapy are incorporated by reference in their entirety.

The term “neoadjuvant therapy” or similar terms means the administration of a therapeutic agent before/prior to the main treatment for the disease. Neoadjuvant therapy has many advantages including that chemotherapy is delivered at the earliest time-point, when the burden of micrometastatic disease is expected to be low; that tolerability of chemotherapy is expected to be better before cystectomy rather than after; and hypothetically that patients with micrometastatic disease might respond to neoadjuvant therapy and thus reveal favorable pathological status determined mainly by negative lymph node status and negative surgical margins.

As used herein, the term “angiogenesis” refers to the growth of new blood vessels. Accordingly, “abnormal angiogenesis” or “aberrant angiogenesis” refers to altered (e.g., increased or decreased) activity of angiogenesis, i.e., any angiogenesis that deviates from the normal process of angiogenesis, such as but not limited to, increased angiogenesis activity in a body, and angiogenesis at an abnormal location of the body. A disease or disorder may be completely caused by abnormal angiogenesis or may be exacerbated by abnormal angiogenesis. Abnormal angiogenesis-related diseases or disorders due to excessive angiogenesis may include, but are not limited to, cancer, tumors, rheumatoid arthritis, psoriasis, rosacea and metastasis of cancerous cells. Abnormal angiogenesis-related disorders due to insufficient angiogenesis may include, but are not limited to, cancer, coronary artery disease, stroke, ulcers and delayed wound healing. In some embodiments, “abnormal angiogenesis” refers to increased or excessive activity of angiogenesis.

As used herein, a “vaccine” is a composition that can be used to elicit protective immunity in a recipient. Thus, after vaccinating a subject with an antigen, the vaccine may prevent or delay disease in a subject exposed to the same or related antigen relative to a non-vaccinated subject, or reduce the severity of the onset. The protective immunity provided by the vaccine can be humoral (antibody-mediated) immunity or cellular immunity, or both. Vaccination can, for example, eliminate or reduce the burden of pathogens or infected cells, or can result in any other measurable reduction in infection. Vaccination can also reduce tumor burden in immunized (vaccinated) subjects.

As used herein, an “adjuvant” or “stimulant” or “immunostimulating agents” or similar refers to a compound that enhances a subject's immune response to an antigen when administered in conjunction with the antigen. adjuvants or immunostimulating agents usually act via their capability to induce an innate immune response. The innate immune system forms the dominant system of host defense in most organisms and comprises barriers such as humoral and chemical barriers including, e.g., inflammation, the complement system and cellular barriers.

As used herein, “affinity capture surface” refers to a material which can be derivatized to form an association with particles such as EVs. The association between EVs and the surface of the material could be either functional bonding, hydrogen bonding, molecular bonding etc. Examples of materials for the affinity capture surface include, but are not limited to, glass (including controlled-pore glass), polymers (e.g., polystyrene, polyurethane, polystyrene-divinylbenzene copolymer), silicone rubber, quartz, latex, a derivatizable transition metal, magnetic materials, silicon dioxide, silicon nitride, gallium arsenide, and derivatives thereof. Except for the reactive sites on the surface, the materials are generally resistant to the variety of chemical reaction conditions to which they may be subjected. In an embodiment, IF EVs are collected on an affinity capture surface for downstream applications such as EM, EDS, IHC, immuno-gold, etc. This concept is related to the spatial collection of EVs.

As defined herein, the term “pharmaceutically acceptable” and its grammatical variations, in its broadest meaning known to a person skilled in the art, means a material capable to be administered to in a vertebrate patient, without undesirable physiological effects such as nausea, dizziness, stomach disorders, fever, allergy and the like.

A “personalized pharmaceutical” shall mean specifically tailored therapies for one individual patient that will only be used for therapy in such individual patient. In an embodiment, personalized pharmaceutical includes personalized vaccines and cellular therapies using patients EVs.

As defined herein, “real-time” or “real time” implies a time to record is taking place simultaneously with occurrence of the event. Is not a recording of something that took place earlier.

As defined herein, “immune remodelling” or similar terms refers to stimulation to repopulate immune cells. The process of immune system remodeling is based on the generation of immune cells in response to a stimuli.

Extracellular vesicles (EVs), or small particles containing proteins, RNA, and other cellular debris, are secreted by cancer cells and could represent an easily accessible source of information about the health and growth of a tumor.

Extracellular vesicles (EVs) from tumor interstitial fluid (IF) are a new category of biomarker that provides real-time functional information about the metabolic and immunologic state of the tumor. In an embodiment, present invention provides a way to isolate these particles rigorously and reproducibly.

Tumor derived extracellular vesicles (EVs) released into the tumor interstitial space enter the lymphatic drainage, are transported to the sentinel lymph node, and eventually reach the blood. Because tumor interstitial fluid (IF) EVs contain molecular information about the current functional state of the tumor, they constitute a powerful new category of EV biomarker for personalized oncology.

Extracellular vehicles (EVs) represent a clinically untapped source of biological information on the health and metabolism of cancer cells.

In an embodiment, EVs play a role in further promoting innate immune responses or inducing adaptive responses following their release from an irradiated tumor. For this reason, a reliable methodology to isolate tumor derived EVs allow for their enhanced characterization with regards to their ability to enhance abscopal effects following radiation treatment.

Table 1 addresses the three dominating sources of variability regarding tumor EV sampling with respect to prior art/existing solution till date and present solution.

TABLE 1
Comparison of existing solution and the present invention

| PROBLEM | EXISTING SOLUTIONS | PRESENT INVENTION |
|---|---|---|
| Clinical Source Material | Blood: Easy to obtain but poor | Core needle biopsy: |
| | recovery and specificity for | Seamlessly integrated into |
| | tumor-derived EVs | pathology workflow; Direct |
| | Bulk tissue in culture: Culture | yield of in situ resident EVs |
| | and enzyme dissociation | in the IF within minutes. |
| | conditions perturb the native EV | Non-destructive: Biopsy can |
| | population: Does not reflect in | still be used for diagnostic |
| | situ state. | pathology. One step kit based |
| | | vacuum extraction protocol |
| | | minimizes pre-analytical |
| | | variability. |
| Purity and Yield | Differential Centrifugation: | Core needle biopsy provides |
| | High yields, low purity | higher-yield source material |
| | Iodixanol gradient: High purity, | and FFF-MALS isolation |
| | low yields | increases purity. |
| Relevant Biomarkers | DNA: Readily measured in | Phosphoproteins: activated |
| | blood EVs but may not contain | cell signaling molecules |
| | tumor specific mutations or | housed in EVs using RPPA; |
| | amplifications | High multiplex > 300 |
| | Protein: Low | phosphoproteins in < 20 |
| | sensitivity and lack of multiplex | microliters; high sensitivity |
| | capability for blood EVs. | (200 picograms/mL) and |
| | | high precision; Signal |
| | | pathway phosphoproteins are |
| | | an unexplored class of EV |
| | | biomarkers reflecting |
| | | functional state of the tumor: |
| | | immediate application for |
| | | individualization of |
| | | neoadjuvant therapy. |

In an embodiment, the fundamental concept of this invention is a method to isolate individual or a collection of the interstitial EVs from a living tissue within a given section and spatial location.

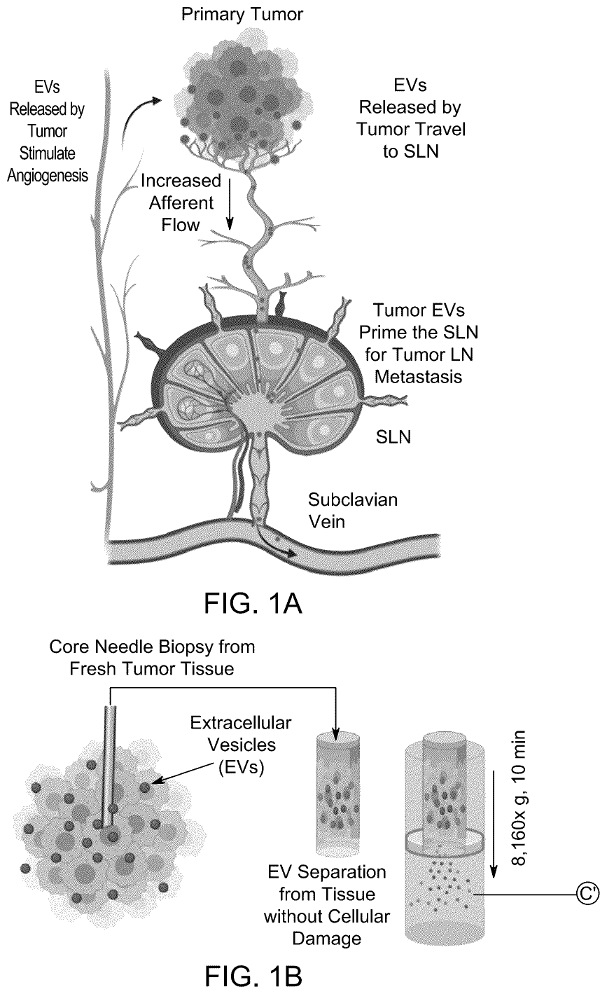

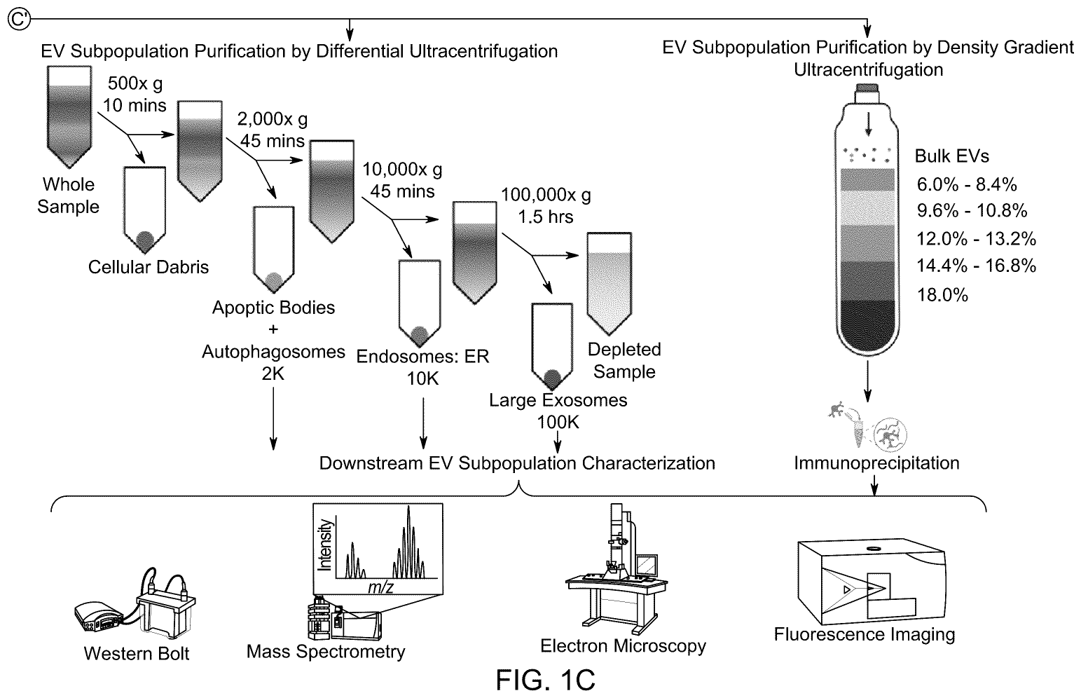

In an embodiment, we developed a novel method and workflow to directly isolate and enrich for EVs shed into, and existing within, the IF in vivo of a syngeneic tumour mass without the use of tissue extraction enzymes (FIG. 1b).

In an embodiment, method does not isolate the interstitial fluid itself, but rather the EVs and microparticles (membranous and non-membranous particles) contained within that specific region of tissue.

In an embodiment, methodology of obtaining IF does not damage the tissue and that this is consistent across experiments. Microscopic images document that the gentle method of EV isolation does not damage or alter the cytomorphology. pre- and post-procedure histology is shown in FIG. 9. The 4T1 murine orthotopic tumors are highly cellular, with a high proportion of solid sheets of carcinoma cells. The size and shape of the carcinoma cell body is unaltered. Furthermore, the fine nuclear chromatin, nuclear membrane and nucleoli, as well as all other morphologic elements are identical for post IF harvesting tumor tissue as compared to freshly excised intact tumors.

Ex-vivo harvesting of resident IF and the EVs contained therein can be done in a few minutes. This includes the short time delay for low-speed centrifugation that harvests approximately 100 to 150 microliters of IF per cubic cm of tumor tissue. This time delay to centrifuge the tumor is one tenth, or less, of the earliest time to observe reactive phosphoprotein changes in ex-vivo tumor tissue prosurvival, hypoxia, and apoptosis pathways.

In an embodiment, purity and yield of proteins from EVs is more than the in vitro method. In an embodiment, purity of proteins from EVs is about 25 times, 40 times, 50 times, 60 times, 70 times or 100 times more than in vitro method. The yield of proteins from EVs is about 25 times, 40 times, 50 times, 60 times, 70 times or 100 times more than in vitro method.

The present invention provides a method and a system to isolate tumor IF directly from excised tissue and allows the same tissue to be processed for routine diagnostic histopathology (FIG. 9). Thus, the molecular characteristics of the tumor derived IF EVs can be correlated directly with the matched tumor tissues. This method increases fidelity, minimizes pre-analytical variability, and provides “ground truth” correlation of the in-vivo state of the tumor cells with the molecular IF EVs.

In an embodiment, present invention provides a construction and use of a technology or method for harvesting resident Interstitial Fluid (IF) Extracellular vesicles (EVs) from living tissue excised from an animal or human.

In an embodiment, present invention provides identification of certain sets of molecules involved in mitochondrial quality control within a cell that are found in EVs present within IF. This new class of EVs and their role in disease provides a new approach for selection of patient optimal treatment and new therapeutic strategies. The invention can be used in a medical hospital setting as a device applied to a tissue biopsy that yields a novel class of information about the in vivo state of the tissue that cannot be determined by conventional histopathologic analysis, genomics or proteomics. This information resides in EVs, which by means of the invention are collected without harming the histopathologic analysis for rendering a diagnosis to provide immediate information regarding the state of the tissue for therapeutic decision making.

In an embodiment, present invention provides measurement of a novel set of mitochondrial-related molecules derived from the resident EVs that provide real-time prognostic or diagnostic information. The new class of EVs can be harvested from freshly excised tissue and be used as biological treatment agents. These EVs contain important molecular information within to serve an important communication function of cells and disease tissue with the immune system to target local or systemic disease.

Prior art searches do not reveal previous public disclosures of vacuum induced one step IF EV collection and do not describe EVs with the mitochondrial molecular composition of the invention.

The present invention utilizes the fresh tissue to obtain a new class of information about the tissue that can be done rapidly and immediately after excision. In an embodiment, EVs could be extracted from the newly excised tissue in less than 24 hours, less than 10 hours, less than 5 hours, one hour, 30 mins, 20 mins, 10 mins, 5 mins, 2 mins, 1 min or less.

In an embodiment, the present invention provides a real-time molecular harvesting of EVs which could be highly relevant to making a clinical decision for that patient. For example, determining which type of molecular targeted therapy, chemotherapy, or immunotherapy is in the best interest for that patient.

The advantage of this system is that a special class of markers is collected from the IF of the tissue in a way that does not harm the cells for routine histopathologic information. Therefore, the molecular information is collected without interfering with routine diagnostic methods, and this new class of molecular information is directly matched to the tissue being examined for diagnosis. The molecular information that comes from this new invention can also be used as a starting point for immune-sensitization or immunotherapy for the patient.

One aspect of the invention is a simple one step for processing a patients tumor tissue biopsy for EVs from the interstitial fluid at the time of tissue collection that does not alter the histomorphologic diagnosis. A new class of molecules within the EV population are harvested to provide therapeutic information and molecules for prognosis. A further application would be for neoadjuvant therapy, where the real-time molecular information derived from the tissue biopsy can be used to determine neoadjuvant therapy to provide to the patient. Moreover, at the time of the surgical excisions of the tumor following neoadjuvant therapy, this invention can be used to monitor if the therapy given to the patient was efficacious and/or associated with complete pathologic remission.

In an embodiment, present invention characterized the in vivo interstitial fluid (IF) content of extracellular vesicles (EVs).

In an embodiment, the present invention used GFP-4T1 syngeneic murine cancer model to study EVs in-transit to the draining lymph node.

In an embodiment, after validating that the IF EVs contained the cell-specific marker (enhanced green fluorescent protein (eGFP), we further purified the EVs and density populations to evaluate their molecular cargo by mass spectrometry, western blotting, chromatography, and ultrastructural morphology (FIG. 1c).

GFP labeling confirmed the IF EV tumor cell origin. Molecular analysis revealed an abundance of IF EV-associated proteins specifically involved in mitophagy and secretory autophagy. A set of proteins required for sequential steps of fission-induced mitophagy preferentially populated the CD81+/PD-L1+ IF EVs; including PINK1 and ARIH1 E3 ubiquitin ligase (required for Parkin-independent mitophagy), DRP1 and FIS1 (mitochondrial pinching), VDAC-1 (ubiquitination state triggers mitophagy away from apoptosis), and VPS35, SEC22b, and Rab33b (vacuolar sorting).

Comparing in vivo IF EVs to in vitro EVs revealed 40% concordance, with an elevation of mitophagy proteins in the CD81+ EVs for both murine and human cell lines subjected to metabolic stress. The export of cellular mitochondria proteins to CD81+ EVs was confirmed by density gradient isolation from the bulk EV isolate followed by anti-CD81 immunoprecipitation, MitoTracker export into CD81+ EVs, and ultrastructural characterization. IF EV export of fission mitophagy proteins has broad implications for mitochondrial function and cellular immunology.

In an embodiment, the present invention created a one-step technology to rapidly isolate fresh tumor IF for molecular characterization of resident IF EVs. The harvesting method, can be done on a biopsy sample or a surgical tissue sample in a few minutes such as less than 60 mins, less than 50 mins, less than 40 mins, less than 30 mins, less than 10 mins or lesser. The method is non-destructive, and retains full tumor tissue integrity for subsequent histopathological diagnostic examination, as shown in FIG. 9.

In an embodiment, the present invention provides a new methodology in animal models and human tumor tissue to reproducibly sample resident tumor EVs within the tumor tissue IF. Tumor-derived EVs can modulate immune recognition of the primary tumor directly through immunomodulatory proteins or indirectly by influencing inflammation, and angiogenesis. Furthermore, the contents of EVs may serve as a rich source of tumor-derived antigens for the next generation of personalized immunotherapies for cancer. For this reason, the methodology to isolate tumor-derived IF EVs reproducibly can provide a new class of rapid real-time biomarker information about the tumor itself compared to searching for the minute subset of tumor derived EVs that eventually reach the blood stream.

In an embodiment, specific phosphoprotein biomarkers in HER2+ breast cancer tissue representing active signaling in the HER2 pathway, including pERBB2 (Y1248), pEGFR (Y1173), pSRC (Y317), pFAK (1576/1577), and pSTAT (Y694). These biomarkers correlate with disease progression and response to anti-HER2 therapies. Comparison of the expression of HER2 pathway phosphobiomarkers between tumor tissue and tumor-derived EVs determines if tumor-derived EVs faithfully recapitulate signatures associated with response to neoadjuvant therapies.

In an embodiment, we isolate EVs directly from the interstitial fluid of mammary tumor biopsies. This protocol, taking advantage of material acquired prior to neoadjuvant therapy, allows for validation with clinically relevant biomarkers (active HER2) while providing material for biomarker discovery.

In an embodiment, we validate the isolation procedures for interstitial fluid EVs using the 4T1-GFP syngeneic mouse breast cancer model. We define the coefficient of variation (CV) and yields of isolation of multiple EV subtypes to validate the procedure.

In an embodiment, we validate our HER2 pathway reverse-phase protein array assay in interstitial EVs using the SKBR3 cell-derived xenograft (CDX) mouse HER2+ breast cancer model. We define coefficient of variation for the reverse phase protein array assay and demonstrate correlation between the expression of phosphoHER2 pathway proteins in the murine tumor versus the tumor-derived EVs.

In an embodiment, we isolate interstitial EV from breast cancer patient biopsy material, then perform our validated HER2 pathway reverse-phase protein array assay to correlate EV expression versus bulk tumor expression.

In an embodiment, we examine the association between EV and tissue HER2 pathway phosphobiomarkers for prediction of clinical response to anti-HER2 therapies. We also perform detailed molecular characterization of the EVs derived from the patient samples as a source for biomarker discovery.

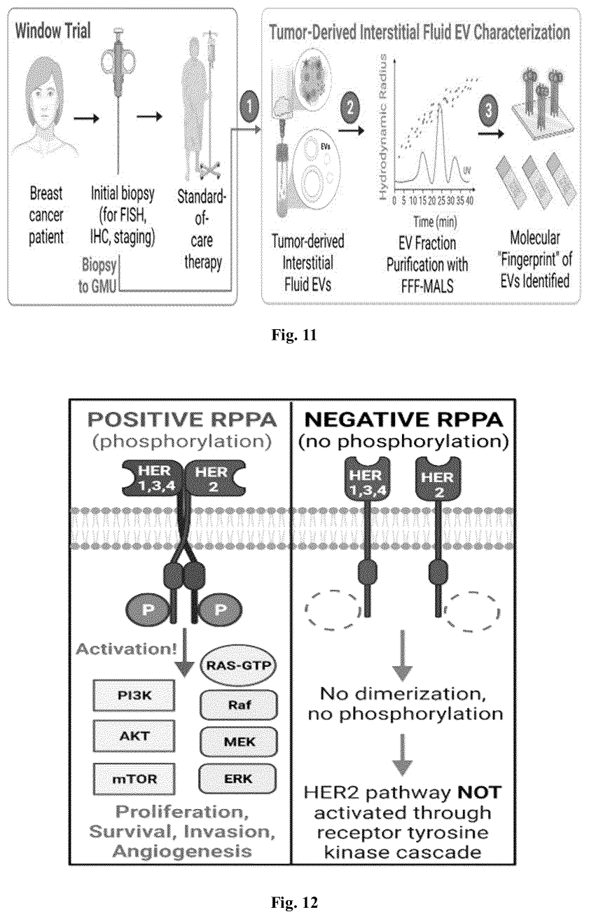

As shown in FIG. 11, the present invention uses clinical material from core needle biopsies collected as part of an ongoing collaboration with Sentara Health during a window clinical trial. Fresh leftover surgical breast cancer tissue specimens at the time of transfer to the surgical pathologist can be sampled for tumor IF EVs in a 2-minute, one-step procedure. The same tissue used for IV EV harvesting can then be fixed or frozen and processed for routine diagnostic histopathology. Thus, the molecular characteristics of the tumor derived IF EVs can be correlated directly with the matched tumor tissues. This workflow increases accuracy and fidelity, minimizes pre-analytical variability, and provides “ground truth” correlation of the in-vivo state of the tumor cells in the microenvironment with the molecular IF EVs. In an embodiment, the present invention provides about activated signaling pathway to inform treatment decision. In an embodiment, present invention provides molecular fingerprint identified for blood-based monitoring (personalized liquid biopsy development). In an embodiment, present invention provides source of tumor derived antigens for next generation personalized immunotherapeutics.

In an embodiment, the present invention provides a reproducible method to isolate EVs directly from the interstitial fluid of a tissue sample such as a breast tumor biopsy, while making clear for the first time if tumor-derived interstitial fluid EV biomarker expression has predictive power for patient therapeutic outcomes.

In an embodiment, the present invention isolates extracellular vesicles directly from biopsy samples such as of breast cancer patients, which avoids some of the complications associated with isolation from blood or from tumors grown in culture.

In an embodiment, the method does not alter the cellular morphology of the tissue sample.

In an embodiment, the purified extracellular vesicles comprise extracellular vesicles can be classified as 2K, 10K, or 100K.

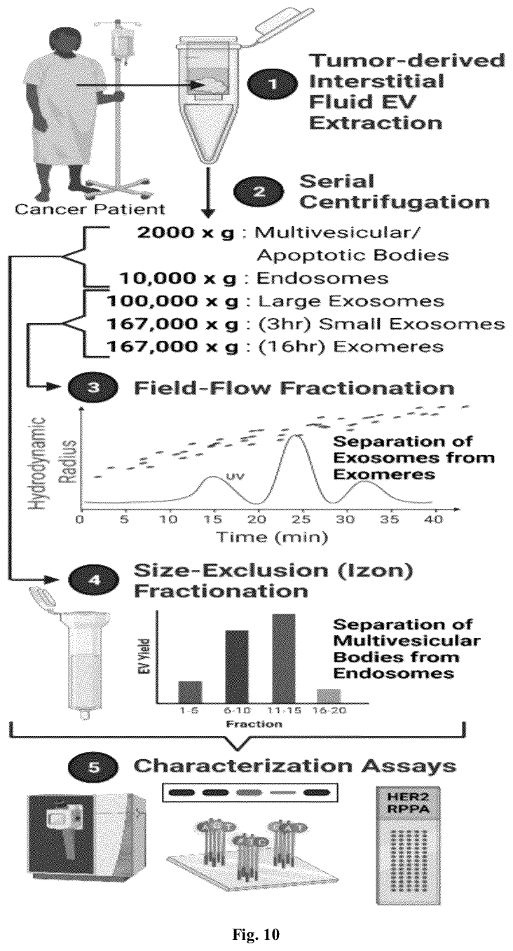

In an embodiment, the present invention analyzes EV purification procedures for reproducibility using field-flow fractionation coupled to multi-angle light scattering (FFF-MALS) in tandem with the centrifugation and size exclusion purifications as shown in FIG. 10.

In an embodiment, the present invention conducts proteomic and miRNA-seq analysis.

In an embodiment, comparing the expression of HER2 pathway phosphobiomarkers between tumor tissue and tumor-derived EVs helps in determining if tumor-derived EVs faithfully recapitulate signatures associated with response to neoadjuvant therapies. Over the course, in an embodiment, the present invention isolates EVs directly from the interstitial fluid of mammary tumor biopsies. This protocol, taking advantage of material acquired prior to neoadjuvant therapy, allows for validation with clinically relevant biomarkers (active HER2) while providing material for biomarker discovery.

In an embodiment, extracellular vesicles (EVs) within tumor interstitial fluid contain a new class of molecular information about the functional state of the tumor. Phosphorylated signal pathway proteins within tumor derived EVs are a window into the activity of tumor cell signaling relevant to neoadjuvant therapy.

In an embodiment, the present invention proposes to validate the rigor and reproducibility of isolation of EVs directly from cancer patient core needle biopsy IF and to correlate the IF EV molecular contents to the expression of cellular signal pathway biomarkers in the matched tumor carcinoma cells.

In an embodiment, tumor IF fluid in animal models is a very rich source of EVs (10¹⁴/mL) and the present invention can verify the tumor derivation of the EVs by a tumor cell specific fluorescent label that is carried into all classes of EVs. For this study, the present invention uses but not limited to field-flow fractionation coupled to multi-angle light scattering (FFF-MALS) to separate different subpopulations of EVs, to be analyzed for their molecular contents by mass spectrometry, RPPA, immunoprecipitation with EV tetraspanin markers, and EM. FFF is capable of handling microliter quantities of EV preparations^{14,15} as obtained from tumor biopsy samples.

In an embodiment, after isolation and purification of EVs from the tissue biopsy, the present invention uses methods such as a reverse phase protein array (RPPA) analysis to determine the activation state of key protein biomarkers.

In an embodiment, the present invention analyzes specific phosphoprotein biomarkers in HER2+ breast cancer tissue samples representing active signaling in the HER2 pathway, including pERBB2 (Y1248; FIG. 12), pEGFR (Y1173), pSRC (Y317), pFAK (1576/1577), and pSTAT (Y694).

In an embodiment, EVs can recapitulate entire active signaling pathways from the originating cell.

In an embodiment, unlike single protein or DNA biomarkers, active signaling pathway analysis of EVs provides a way to uniquely identify the EV as both tumor-derived and biologically active, with insights into the tumor biology. By isolating tumor-derived EVs and comparing the expression of HER2 pathway phosphobiomarkers to bulk tissue, the present invention is able to discover associations between the proteomic content of tumor-derived EVs and a specific outcome following treatment, as shown in Table 2. Table 2 represents diverse pathways.

TABLE 2
Proteins identified in interstitial EVs.

| PATHWAY | PROTEIN IDENTIFIED |
|---|---|
| Autophagy | Sequestosome-1 (p62) |
| | Microtubule-associated proteins 1A/1B light chain 3B |
| | (LC3b) |
| Mitochondrial | Mitochondrial import receptor subunit TOM20 (TOM20) |
| Health | Mitochondrial import receptor subunit TOM34 (TOM34) |
| | Voltage-dependent anion-selective channel protein |
| | (VDAC1)Cytochrome c oxidase subunit 4 (COX4) |
| Immune | Programmed cell death ligand 1 (PD-L1) |
| Recognition | CD44 antigen (CD-44) |

In an embodiment, the present invention isolates tumor derived EVs directly from the interstitial fluid using a tissue sample. This isolation does not damage the tissue or the cellular diagnostic histomorphology (FIG. 9), with no evidence of increased apoptosis following the 2-minute collection. In the present invention EV isolation harvests resident tumor IF EVs directly from a biopsy sample ex-vivo in minutes, thus avoiding the serious preanalytical variability caused by cold ischemia. Unlike isolation from blood or other body fluids, we can achieve high yield of EVs that can be proved to originate in the tumor tissue in animal models.

The invention disclosed contains a novel method to extract or harvest EVs from the interstitial space of a tissue sample. For example, during a patient tumor biopsy, the tumor would be subjected to the negative pressure and/or inertial difference to harvest the EVs from the tumor interstitial space. The tools to collect the EVs could be contained in a kit that would have the necessary vials, tubes, and buffers to harvest the EVs in one step. The tissue can be subsequently immediately processed for diagnostic histopathology. This would be utilized in a routine manner that could easily be integrated into the current tissue analytical methods post-excision.

The invention disclosed contains novel methods to analyze the EVs that resided within a tissue sample. For example, after harvesting of the EVs from the in situ tissue interstitial fluid, the EVs could be analyzed for metabolic stress markers, active phosphorylation signal pathways, and check-point markers for insights into tumor immune suppression that we have identified as being markers to monitor the tissue status. This information provides real-time prognostic and diagnostic information derived from the tissue sample itself which current means of analysis either fail to achieve, lack the sensitivity, require increased costs, increase analytical processing time increase analytical methods, to assess a tumor fail to provide information of the state of the tissue. Therefore, the invention disclosed contains numerous methodologies which could be licensed to companies for diagnostic/prognostic purposes.

The invention disclosed herein, comprises new methods of immune sensitization by using a patient's tissue EVs that are determined to contain markers (check-point inhibitors, autophagy/mitophagy constituents) to aid in immune modulation to a disease. These isolated EVs could be injected back into a patient and travel to the local lymph node for immune activation. Additionally, these EVs could be analyzed for markers related to cell survival through the elaboration of the EVs as a communication mechanism to neighbor cells. After this analysis, the patient could be treated to prevent the said communication mechanism either at the output or input of communication.

The invention disclosed herein, is not limited to tumor or cancer biology. The method to collect EVs and the methods to analyze the EVs can be applied to other non-neoplastic diseases such as neurodegenerative, cardiac, and/or musculoskeletal diseases.

In an embodiment, EVs can travel intact to the draining lymph node, where they can either prime the immune system or suppress the immune system, depending on size and molecular cargo, demonstrating their biological activity.

In an embodiment, we show unique evidence that tumor-derived EVs contain a more complicated proteomic complement than originally thought, including proteins involved in autophagy, mitochondrial health, and immune recognition (Table 2). This cargo demonstrates the importance of pathway analysis to understand the biology of the tumor cells and provides a fresh resource for biomarker discovery.

In an embodiment, the present invention probes isolated tumor-derived interstitial fluid EVs for additional protein biomarkers in the mitophagy pathway and discovers if these biomarkers contribute to innate immune activation responsible for the abscopal effect.

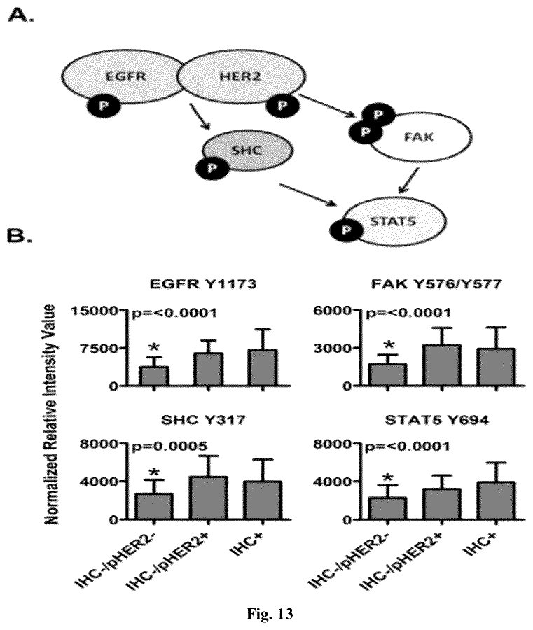

In an embodiment, the present invention probes HER2+ pathway phosphorylation sites via reverse phase protein arrays (RPPA) and predicts responses to anti-HER2 therapies. As shown in FIG. 13, we have demonstrated that four different phosphoprotein sites in the HER2 pathway have significantly increased intensity in patient samples that are positive via immunohistochemistry (IHC+) for HER2 as compared to those samples that were IHC−.

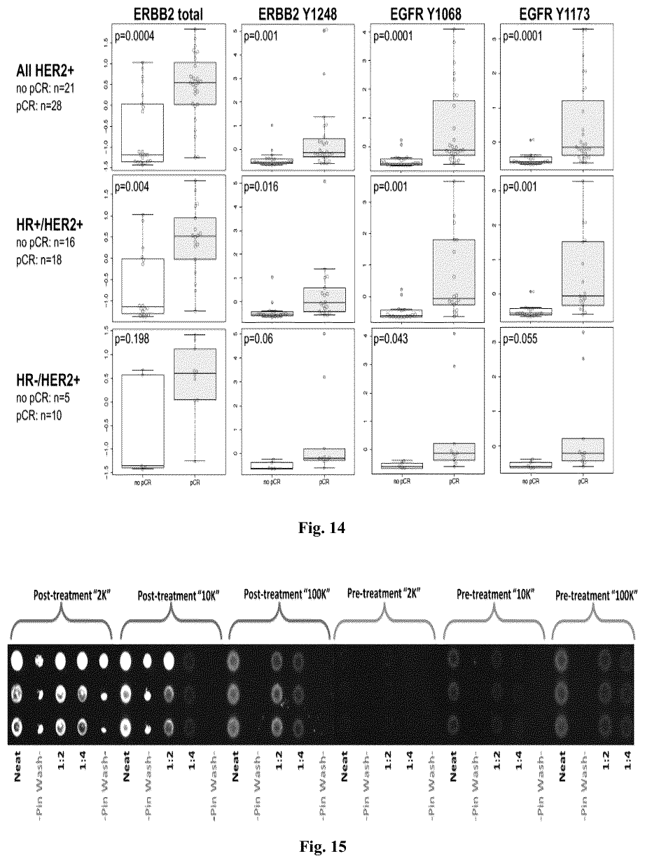

Additionally, as shown in FIG. 14 we find that RPPA measurement of HER2 (ERBB2) total protein, one HER2 phosphorylation site as well as two different phosphorylation sites on it's dimeric partner EGFR, can be used to predict pathological complete response (pCR) to anti-HER2 therapy. Advanced breast cancer patients were enrolled in the I-SPY 2 clinical trial¹⁷, an adaptive clinical trial in which pCR (primary endpoint) is evaluated following neoadjuvant therapy of new treatments in addition to standard of care. Bayesian adaptive randomization allows for each patient to move into arms in which they are predicted to have the most benefit; new treatments that improve pCR graduate. Following patients in the T&P treatment arm (administration of T-DM1 (T), a conjugate of the anti-HER2 mAb trastuzumab and the microtubule assembly inhibitor emtansine, in combination with pertuzumab (P) an anti-HER2 mAb) showed that T&P treatment improved pCR in HER2+ subtypes such that T&P graduated from the trial in all HER2+ subtypes.

In an embodiment, RPPA assays previously used for tissue lysates can be adapted for use with EV subpopulations. For example, as shown in FIG. 15, EVs from ovarian cancer line OVCAR8 show evidence of upregulation of phosphobiomarkers by RPPA.

In an embodiment, the present invention, can be enabled to probe isolated tumor-derived interstitial fluid EVs for additional protein and RNA biomarkers in the mitophagy pathway to better understand the energy requirements of cancer cells, and how to exploit these requirements for cancer detection.

In an embodiment, EVs isolated from the interstitial fluid of tumour contain unique markers as compared to cells grown in culture. Untargeted proteomics and western blots show complicated biological pathways, including an unreported secretory mitophagy pathway.

In an embodiment, TEM images, proving the “100K” EVs are homogenous and morphologically similar to those large exosomes described in literature, confirming the identity of these particles under study. To derive meaningful biological information from the complex cargo identified, the present invention proposes to focus on clinically relevant phosphoproteins.

In an embodiment, extracted tumor EV subpopulation material serves as a source of biomarkers for further probing of cancer biology, with CV<15% between isolations intra-mouse; CV<25% between isolations inter-mouse.

In an embodiment, interstitial EVs are isolated with a centrifugal protocol.

In an embodiment, RPPA analysis shows complete four HER2 pathway endpoints with HER2+ mouse mammary tumor tissue lysates, each subpopulation of interstitial EVs, and negative EV subpopulation controls from 4T1-GFP mouse mammary tumors.

In an embodiment, CV<15% between isolations of a single tumor; ROC AUC>0.75 for RPPA prediction of HER2 status.

In an embodiment, HER2 pathway biomarker with additional HER2 pathway phosphobiomarkers from FIG. 13 (pSRC Y317, pSTAT Y694, pFAK Y1576/1577) can be added.

In an embodiment, tumor-derived interstitial fluid EVs contain HER2 pathway phosphor-endpoints that successfully predict the HER2 status of the bulk tumor.

In an embodiment, ROC AUC>0.75 for prediction of HER2 status; CV<15% between control proteins.

The advantage of a single protein calibrator versus standardization on total protein is that single protein calibrators allow for the same calibrator to be used across techniques that either are not able to be normalized to total protein (such as immunohistochemistry) of those in which single-protein calibrators are preferred (such as mass spectrometry). However, if no single protein can be identified as a suitable calibrator, normalization can be conducted based on total protein abundance.

If the HER2 samples fall below the expected 20% of total samples collected, in this case, higher AUC values could be confidently determined. However, we have estimated expected AUC very conservatively (AUC=0.75 whereas literature from a lower sensitivity biofluid found AUC=0.7931) such that even if recruitment falls short, we will likely be able to compensate if predictive power is greater than AUC=0.75.

In an embodiment, a new resource of patient-derived biomarkers from validated tumor derived EVs can be described, and the ability of HER2 phosphobiomarker levels, to directly predict bulk tissue phosphobiomarker levels, can be determined.

Expression of one or more HER2+ pathway phosphobiomarkers in tumor-derived interstitial EVs correlate with pathological complete response (pCR) to anti-HER2 therapy.

Human tumor tissue is highly heterogenous, with inclusion of necrotic regions, immune infiltrate, and stromal cells, in additional to different clonal populations of the tumor cells themselves. This means that correlation of biomarkers with bulk tissue could be complicated by the presence of non-tumor cells or tumor cells at different stages. In this case, we can use laser capture microdissection (LCM) to isolate pure cell populations.

To understand the importance of the global functional consequences of the separate EV categories, we studied their effects on SLN modulation of tumour growth, angiogenesis, and distant metastasis. For these functional comparisons, we employed our previously developed model of delivering isolated EVs directly in order to prime the SLN for cancer cell challenge (Longo et al., 2009; Luchini et al., 2008; Popova et al., 2015; Tamburro et al., 2011; Teunis et al., 2017).

In one embodiment, it has been hypothesized that secretory autophagy contributes to the growth of the tumour (Dupont et al., 2011).

In an embodiment, secretory autophagy has never been studied in IF (Morgan et al., 2019; New & Thomas, 2019); therefore, we studied the presence of molecular mediators specific to secretory autophagy (SEC22b and RAB33b) compared to canonical autophagy machinery (LC3-I/II and p62) (Morgan et al., 2019; New & Thomas, 2019).

Further, there is a significant lack of knowledge concerning the role of cellular EV production and the mitophagy process. Considering mitochondrial function and energy metabolism are crucial components of normal and diseased tissue cell function, combined with the recent findings that mitochondrial proteins are present within EVs (Jang et al., 2019; Macleod, 2020). In an embodiment, we evaluated the EVs for specific molecular complex of mitophagy.

In an embodiment, we verified our mitophagy characterization using EV density gradient separation followed by immunoprecipitation using EV specific markers followed by western blot for a key mitophagy initiator (PINK1). Additionally, we fluorescently labelled active mitochondria and tracked the fluorescent dye presence in the released EVs.

In an embodiment, molecular characterization of the IF revealed that a specific population of EVs contained a large set of proteins all specifically associated with the sequential steps of mitophagy fission and mitophagy, not mitophagy fusion, nor mitochondria-associated apoptosis.

Altogether, these results revealed a notable functional difference in the EV subpopulations, and provide new insights about the biology of secretory autophagy and extracellular mitophagy components shed within IF EVs.

Abscopal Effect

In an embodiment, classes of EVs released from tumor following irradiation facilitate immune remodeling in the draining lymph node and complement the innate immune recognition following interferon signaling that plays a role in abscopal effect. This is in line with existing literature emphasizing the role of mitophagy and mitochondrial components in dampening or raising innate immune responses, and our own preliminary data finding large numbers of mitophagy related proteins in some classes of tumor immunomodulation by this EV class derived interstitial Fluid EVs, along with early evidence of immunomodulation by this EV class.

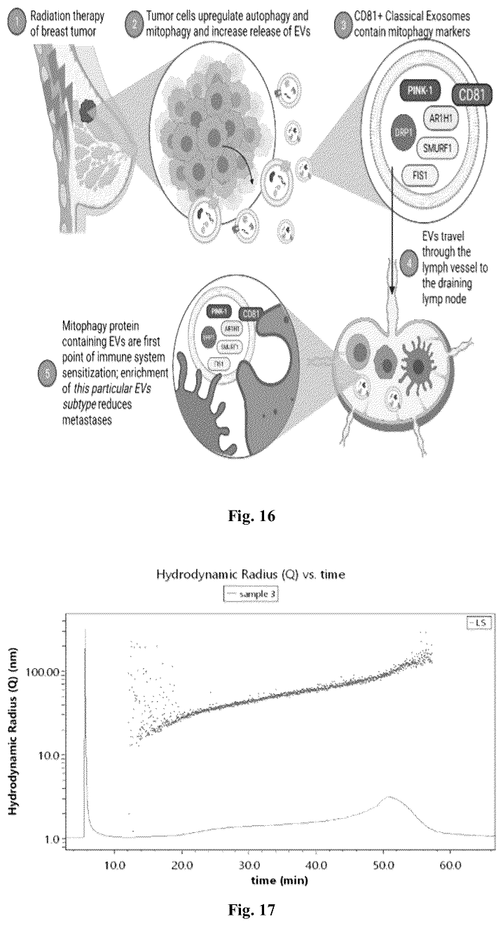

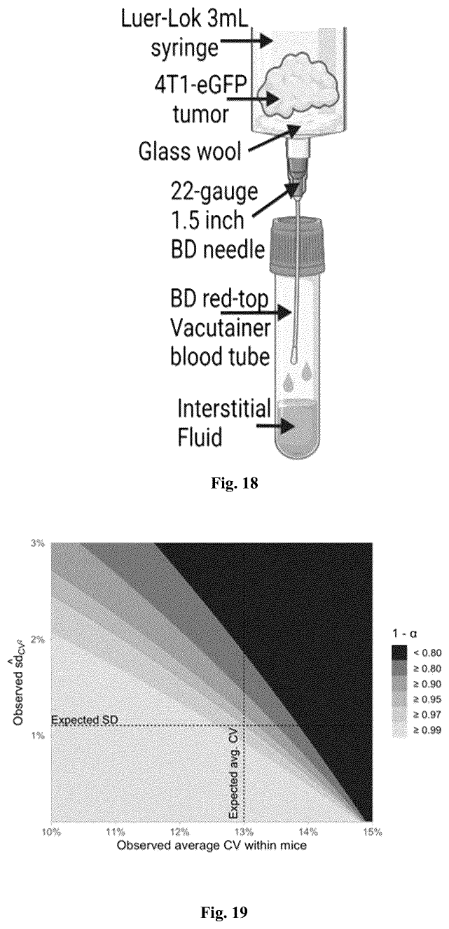

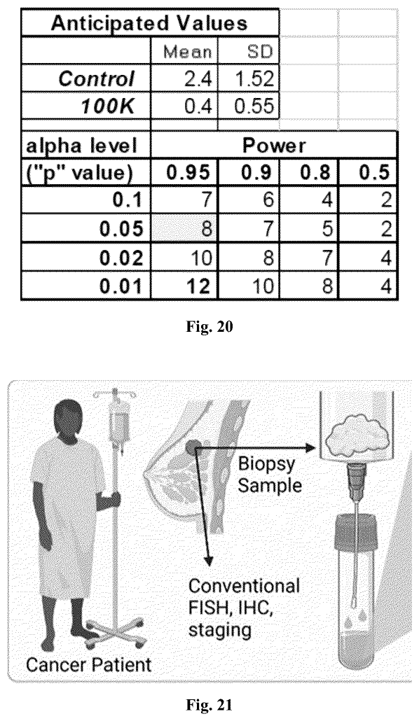

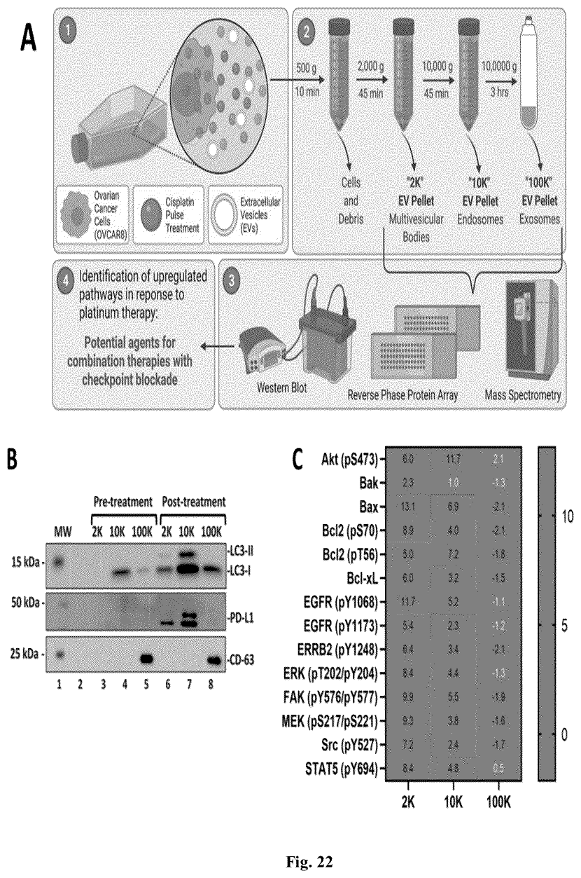

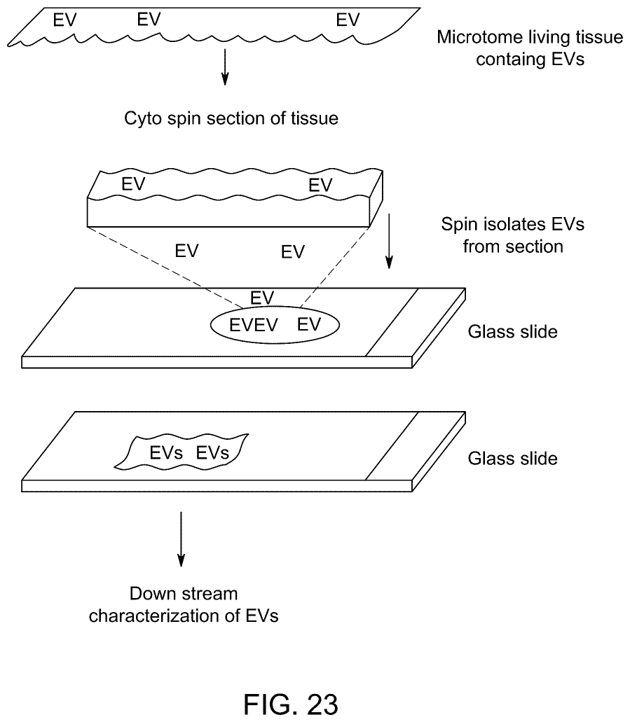

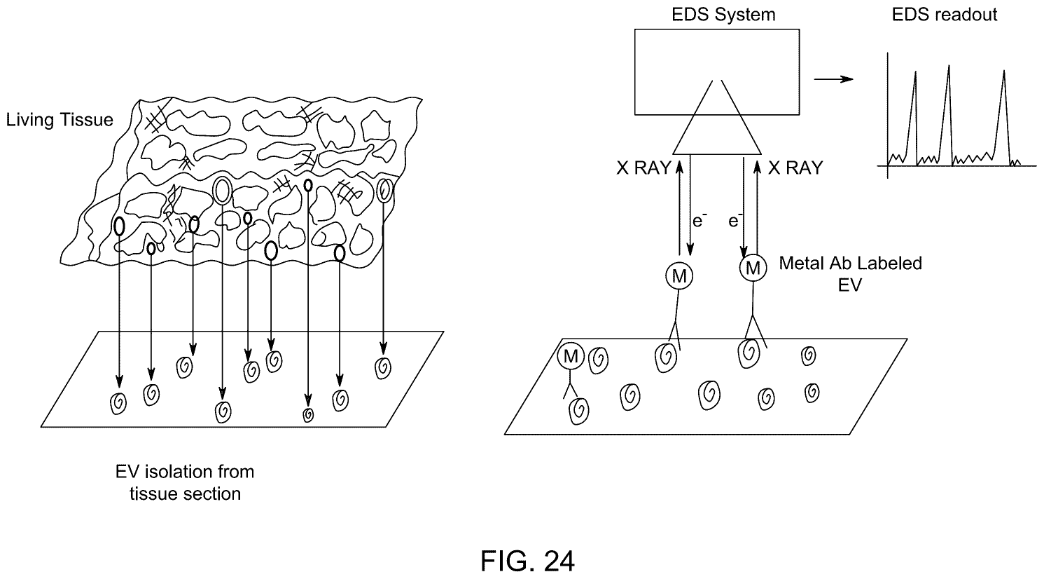

In an embodiment, one class of EVs (PINK+ ARIH1+ CD81+ 100 nm classical exosomes) released from tumor tissue following irradiation facilitates immune remodelling in the draining lymph node and complements the innate immune recognition following interferon signalling that plays a role in abscopal effect (FIG. 16). This is in line with existing literature emphasizing the role of mitophagy and mitochondrial components in dampening or raising innate immune responses, and our preliminary data finding large numbers of mitophagy-related proteins in the CD81+ class of tumor-derived IF EVs, along with early evidence of immunomodulation by this EV class.

The abscopal effect is an action-at-a-distance phenomena. For example: in breast cancer, when a primary breast tumor is treated with radiation therapy, non-irradiated distant metastases can also shrink. Furthermore, it has been shown that early radiation treatment of ductal carcinoma in situ (DCIS, “Stage 0” breast cancer) after lumpectomy can reduce the rate of recurrence. However, while the abscopal effect has been long known, the mechanism is not completely understood.

The present invention proposes release of tumor antigens to the lymph node to promote the abscopal effect though it is rare and unlikely due to existing tumor tolerance. Mitochondrial components and autophagy also play a key role in this process. Activation of autophagy in irradiated tumor cells reduces type 1 interferon secretion, rendering the tumor less immunologically hot; inhibition of autophagy with the inhibitor chloroquine improves response to radiation therapy in mice. Furthermore, the induction of interferon secretion was shown to be directly tied to cytosolic mitochondrial DNA (mtDNA) found in the irradiated tumor; autophagy reduces this accumulation of cytosolic mtDNA by recycling permeabilized mitochondria. The specific recycling of mitochondria (mitophagy) recruits a number of unique proteins such as serine-threonine kinase PINK1 and the E3 ubiquitin ligase Parkin in Parkin-dependent mitophagy, or alternative ubiquitin ligases like ARIH1 in parkin-independent mitophagy.

In an embodiment, radiation increases EV release, and these EVs could play a supporting role in modulating the abscopal effect; however, different EV subtypes can display immunosuppressive or immunostimulatory effects. Direct monitoring of mitophagy proteins or their release in EVs and the specific EV populations into which these proteins segregate in the context of the abscopal effect has not been studied to date.

In an embodiment, we validate the rigor and reproducibility of isolation of IF EVs directly from excised tumor samples and explore the isolated IF EVs in mouse models of the abscopal effect. We have found that tumor IF in animal models is a very rich source of EVs (10¹⁴/mL) and the tumor derivation of the EVs by a tumor cell specific fluorescent label that is carried into all classes of EVs.

In an embodiment, EVs, beyond just carrying unneeded molecular detritus, can in fact recapitulate entire active signaling pathways from the originating cell.

In an embodiment, we isolate tumor derived EVs directly from the interstitial fluid, allowing for harvest of EVs from a resected tumor sample while retaining tissue integrity for subsequent histopathological examination. Tumor derived EVs released into the interstitial space enter the lymphatic drainage, are transported to the sentinel lymph node, and eventually reach the blood. Using differential ultracentrifugation followed by immunoprecipitation and density gradient isolation, we have shown that we can isolate highly pure CD81+ PINK1+ 100 nm EVs, which are classical exosomes containing mitophagy markers.

In an embodiment, the present invention provides a rigor and reproducibility of extraction of interstitial fluid extracellular vesicles using 4T1-GFP syngeneic mouse model. Interstitial EVs from the tumors are isolated, fractionated, and purified using our in-house protocols.

In an embodiment, the present invention provides a rigor and reproducibility of EV subpopulation isolation protocols and biomarker discovery of validated extractions. Groups of N=20 mice are sufficient to validate CV % to 95% confidence within expected experimental error. Following validation of extraction procedure, the isolated material is used for biomarker identification/validation with the following: untargeted mass spectrometry, western blot for markers of mitophagy (PINK1, ARIH1, TOM20, DRP1), and RPPA for phosphobiomarkers (Bcl2 pT56, pS70).

In an embodiment, the present invention provides isolation and characterization of radiation-treated 4T1 IF EVs.

In an embodiment, the present invention provides mouse models of the EV abscopal effect with and without EV irradiation. 4T1 mouse model of the EV abscopal effect in Balb/c mice using irradiated 100K IF EVs (N=10 mice/group). We compared the number of lung metastasis following EV pretreatment using both irradiated and non-irradiated 100K EVs. Two days following EV pretreatment, mice are challenged with 1×10⁶ 4T1 cells/mouse at the same location and monitored for spontaneous tumor growth. Validation Study set for HER2 pathway biomarker quantification via RPPA in EV subpopulations and matched bulk tissue: TS/A mouse model of the EV abscopal effect in Balb/c mice using irradiated 100K IF EVs (N=10 mice/group): In order to ensure that responses are not due to unique qualities of the 4T1 cells, we conducted identical immune priming experiments with an alternative syngeneic breast cancer model, using TS/A mouse breast cancer cells.

In an embodiment, the present invention compares EV pretreated using both irradiated and non-irradiated 100K EVs, with conventional in-vivo radiation treatment to reduce the number of distant metastases.

In an embodiment, mitophagy-protein containing EVs show reductions in distant metastases following cancer cell challenge in the 4T1 triple negative breast cancer (TNBC) mouse model.

In an embodiment, we have developed extraction of interstitial fluid EVs (IF EVs) directly from labelled tumor tissue in a mouse model, proving the origin of the EVs as well as providing a concentrated preparation of EVs to use for characterization.

In an embodiment, we characterize the IF EVs, both with and without radiation treatment, via mass spectrometry, Western blot, and reverse phase protein array (RPPA).

In an embodiment, injecting the characterized IF EVs, with or without radiation treatment, into naïve mice to determine if such treatment can induce an abscopal like effect to reduce the number of distant metastases following cha cell lines in two different syngeneic mouse models of breast cancer challenge with breast cancer.

In an embodiment, extracted tumor IF EV subpopulation material serves as a source of biomarkers for characterization.

In an embodiment, 100K IF EVs stimulate an abscopal-like effect, reducing distant metastasis, are determined in two different syngeneic mouse models of breast cancer. Furthermore, the role of irradiation on 100K IF EV contents/quantities and differences in immunopriming potential could be elucidated. One potential pitfall could be that the 100K IF EV pretreatment successfully reduced metastases in one mouse model but not the other. To understand the generalizability of the results, we conducted the experiments with a third syngeneic mouse model of breast cancer using E0771.lmb38 cells and C57Bl/6 mice. In addition, immunohistochemical analysis of the spontaneous metastases and surrounding tissues as well as the lymph nodes can be used to compare the levels of immune infiltrate with mouse-specific markers to understand differences leading to different responses.

In an embodiment, the present invention provides a novel use of tumor-derived IF EVs, offering a different approach to understanding the abscopal effect based on EV communication with the sentinel lymph node. Furthermore, we confirm and extend previous literature by providing mechanistic insights into the role of autophagy and mitophagy proteins in modulating the abscopal effect. Additional mouse experiments determined the therapeutic effect of these EVs in a post-treatment rather than pre-treatment system, using knockout cell lines to probe the role of unique markers that modulate the response.

In an embodiment, the present invention provides a method for collecting extracellular vesicles resident in the tissue interstitial space comprising a fresh ex-vivo tissue sample obtained from an animal and/or human sample and subjecting the sample to an inertial and/or atmospheric pressure differential to cause the Extracellular vesicles present in the interstitial space to be discharged from whole tissue volume under conditions such that the tissue cells are not ruptured or disrupted so that the tissue remains intact for tissue histopathologic examination.

In an embodiment, system does not require any physical method or tool to pierce, puncture, or damage the cellular structure of the tissue sample to collect the extracellular vesicles resident within the interstitial space.

In an embodiment, system would comprise of a chamber containing a high porosity matrix for the tissue to rest upon which is connected to a vacuum pressure collection vessel such as a closed vacuum vial.

In an embodiment, collection vessel for the interstitial fluid from the tissue sample would contain a preservative to reduce enzymatic degradation of the sample.

In an embodiment, system could be utilized on a variety of tissue biopsy samples for many neoplastic and non-neoplastic diseases including but not limited to: neurodegenerative diseases, cardiac diseases, cancer, and muscle-nerve function/dysfunction disorders.

In an embodiment, extracellular vesicles collected can be administered as an immunotherapy to overcome tumor suppression.

In an embodiment, the present invention provides a means of conducting sanitizing a host by immune sensitization by collecting extracellular vesicles from the interstitial space of a tissue sample that has the following characteristics including but not limited to: immune check-point inhibitors and autophagy/mitophagy pathway constituents, so that the collected extracellular vesicles are exposed to the immune system or constitute an immune modulation by administrating EVs to travel to the local lymph node.

In an embodiment, the present invention provides a means of evaluating the real-time metabolic state of a tissue to collect the interstitial EVs and evaluate the mitophagy-associated proteins contained within to make a judgement to determine if the tissue sample has a specific metabolic stress such as response to treatment.

In an embodiment, the present invention provides a method to modulate the pathways that regulate EV cell production to contain one or more mitophagy-associated proteins in the following list but not limited to PINK1, PTEN-induced kinase 1; DRP1, dynamin-related protein 1; FIS1, mitochondrial fission 1 protein; ARIH1, Ariadne RBR E3 Ubiquitin Protein Ligase 1; HUWE1, HECT, UBA And WWE Domain Containing E3 Ubiquitin Protein Ligase 1; SMURFI, Smad ubiquitin regulatory factor 1; p62, sequestome 1; LC3, Microtubule-associated protein 1A/1B-light chain 3; PE, phosphatidylethanolamine; Sec22B, SEC22 Homolog B, Vesicle Trafficking Protein; Rab33B, Ras-Related Protein Rab-33B; HSP90/CDC37, 90 kDa heat shock protein and Cell Division Cycle 37; VDAC, Voltage-dependent anion-selective channel 1; TOM34, Translocase Of Outer Mitochondrial Membrane 34; VPS-35, Vacuolar protein sorting ortholog 35, to treat the following diseases: neurodegenerative, cardiac, musculoskeletal disorders, and cancer diseases.

In an embodiment, the present invention provides a means of treating a neurodegenerative, muscular dysfunction, or neoplastic disorder comprising a blockade of the communication process between two cells where one elaborates EVs containing components of the mitophagy pathway and said EV(s) is/are taken up by a recipient cell that contributes to the state of the disease.

In an embodiment, such a therapy could block at the output or the input.

In an embodiment, components are related to the fission pathway of mitophagy.

In an embodiment, provides evaluation of the phosphorylated state of the cellular signaling pathways including HER-2, ERB family receptors, Tyrosine kinase receptors, G protein receptors, Immune related cell surface signaling such that the phosphorylated state provides information about the activity of the signaling pathway between the cell that donates the EV.

Composition

In an embodiment, composition is suitable for administration to a human being in a medical setting. Preferably, a pharmaceutical composition is sterile and produced according to GMP guidelines.

In an embodiment, the composition may be microparticles, nanoparticles, lipid emulsions or similar formulations. The particles may be include but are not limited to a polymer, a metal or a ceramic.

In an embodiment, the composition may also include one or more adjuvants. Adjuvants are substances that non-specifically enhance or potentiate the immune response (e.g., immune responses mediated by CD8-positive T cells and helper-T (TH) cells to an antigen, and would thus be considered useful in the medicament of the present invention. Suitable adjuvants include, but are not limited to, 1018 ISS, aluminum salts, AMPLIVAX®, AS15, BCG, CP-870,893, CpG7909, CyaA, dSLIM, flagellin or TLR5 ligands derived from flagellin, FLT3 ligand, GM-CSF, IC30, IC31, Imiquimod (ALDARA®), resiquimod, ImuFact IMP321, Interleukins as IL-2, IL-13, IL-21, Interferon-alpha or -beta, or pegylated derivatives thereof, IS Patch, ISS, ISCOMATRIX, ISCOMs, Juvlmmune®, LipoVac, MALP2, MF59, monophosphoryl lipid A, Montanide IMS 1312, Montanide ISA 206, Montanide ISA 50V, Montanide ISA-51, water-in-oil and oil-in-water emulsions, OK-432, OM-174, OM-197-MP-EC, ONTAK, OspA, PepTel® vector system, poly(lactid co-glycolid) [PLG]-based and dextran microparticles, talactoferrin SRL172, Virosomes and other Virus-like particles, YF-17D, VEGF trap, R848, beta-glucan, Pam3Cys, Aquila's QS21 stimulon, which is derived from saponin, mycobacterial extracts and synthetic bacterial cell wall mimics, and other proprietary adjuvants such as Ribi's Detox, Quil, or Superfos. Adjuvants such as Freund's or GM-CSF are preferred. Several immunological adjuvants (e.g., MF59) specific for dendritic cells and their preparation have been described previously. Also, cytokines may be used. Several cytokines have been directly linked to influencing dendritic cell migration to lymphoid tissues (e.g., TNF-), accelerating the maturation of dendritic cells into efficient antigen-presenting cells for T-lymphocytes (e.g., GM-CSF, IL-1 and IL-4) (U.S. Pat. No. 5,849,589, specifically incorporated herein by reference in its entirety) and acting as immunoadjuvants (e.g., IL-12, IL-15, IL-23, IL-7, IFN-alpha. IFN-beta).

In an embodiment, the composition may incorporate adjuvant as disclosed in U.S. Ser. No. 11/427,614B2, which is incorporated by reference herein.

In an embodiment, composition is used for parenteral administration, such as subcutaneous, intradermal, intramuscular or oral administration. For this, the peptides and optionally other molecules are dissolved or suspended in a pharmaceutically acceptable, preferably aqueous carrier. In addition, the composition can contain excipients, such as buffers, binding agents, blasting agents, diluents, flavors, lubricants, etc. The peptides can also be administered together with immune stimulating substances, such as cytokines.

An extensive listing of excipients that can be used in such a composition can be, for example, taken from A. Kibbe, Handbook of Pharmaceutical Excipients (Kibbe, 2000).

Effective dosages and schedules for administering the composition may be determined empirically, and making such determinations is within the skill in the art. Those skilled in the art will understand that the dosage of composition that must be administered will vary depending on, for example, the subject that will receive the antibody, the route of administration, the particular type of antibody used and other drugs being administered. A typical daily dosage of the antibody used alone might range from about 1 mg/kg to up to 100 mg/kg of body weight or more per day, depending on the factors mentioned above.

In an embodiment, the composition may be used as a vaccine. It is important to realize that the immune response triggered by the vaccine according to the invention attacks the a disease such as cancer in different cell-stages and different stages of development. Furthermore, different cancer associated signaling pathways are attacked. This is an advantage over vaccines that address only one or few targets, which may cause the tumor to easily adapt to the attack (tumor escape).

In an embodiment, a composition comprising extracellular vesicles (EVs) comprising proteins that are components of mitochondria produced during cellular mitophagy of a cell, wherein the mitophagy associated mitochondrial proteins reflect/represent/is a measure of/provides/external indicator/surrogate end-point of the cellular metabolic or immunological state of a treatment.

In an embodiment, present invention is further directed at a kit comprising: (a) a container containing a pharmaceutical composition as described above, in solution or in lyophilized form; (b) optionally a second container containing a diluent or reconstituting solution for the lyophilized formulation; and (c) optionally, instructions for (i) use of the solution or (ii) reconstitution and/or use of the lyophilized formulation.

The kit may further comprise one or more of (iii) a buffer, (iv) a diluent, (v) a filter, (vi) a needle, or (v) a syringe. The container is preferably a bottle, a vial, a syringe or test tube; and it may be a multi-use container. The pharmaceutical composition is preferably lyophilized.

Kits of the present invention preferably comprise a lyophilized formulation of the present invention in a suitable container and instructions for its reconstitution and/or use. Suitable containers include, for example, bottles, vials (e.g. dual chamber vials), syringes (such as dual chamber syringes) and test tubes. The container may be formed from a variety of materials such as glass or plastic. Preferably the kit and/or container contain/s instructions on or associated with the container that indicates directions for reconstitution and/or use. For example, the label may indicate that the lyophilized formulation is to be reconstituted to peptide concentrations as described above. The label may further indicate that the formulation is useful or intended for subcutaneous administration.

The container holding the formulation may be a multi-use vial, which allows for repeat administrations (e.g., from 2-6 administrations) of the reconstituted formulation. The kit may further comprise a second container comprising a suitable diluent (e.g., sodium bicarbonate solution).

The kit may further include other materials desirable from a commercial and user standpoint, including other buffers, diluents, filters, needles, syringes, and package inserts with instructions for use.

Kits of the present invention may have a single container that contains the formulation of the pharmaceutical compositions according to the present invention with or without other components (e.g., other compounds or pharmaceutical compositions of these other compounds) or may have distinct container for each component.

Preferably, kits of the invention include a formulation of the invention packaged for use in combination with the co-administration of a second compound (such as adjuvants (e.g. GM-CSF), a chemotherapeutic agent, a natural product, a hormone or antagonist, an anti-angiogenesis agent or inhibitor, an apoptosis-inducing agent or a chelator) or a pharmaceutical composition thereof. The components of the kit may be pre-complexed or each component may be in a separate distinct container prior to administration to a patient. The components of the kit may be provided in one or more liquid solutions, preferably, an aqueous solution, more preferably, a sterile aqueous solution. The components of the kit may also be provided as solids, which may be converted into liquids by addition of suitable solvents, which are preferably provided in another distinct container.

The container of a therapeutic kit may be a vial, test tube, flask, bottle, syringe, or any other means of enclosing a solid or liquid. Usually, when there is more than one component, the kit will contain a second vial or other container, which allows for separate dosing. The kit may also contain another container for a pharmaceutically acceptable liquid. Preferably, a therapeutic kit will contain an apparatus (e.g., one or more needles, syringes, eye droppers, pipette, etc.), which enables administration of the agents of the invention that are components of the present kit.

The present formulation is one that is suitable for administration of the peptides by any acceptable route such as oral (enteral), nasal, ophthal, subcutaneous, intradermal, intramuscular, intravenous or transdermal. Preferably, the administration is s.c., and most preferably i.d. administration may be by infusion pump.

since the EVs of the invention were isolated from tumour tissue, the medicament of the invention is preferably used to treat the cancer.

Diagnostic Method

The devices or methods described herein can be employed in various diagnostic applications for detecting and capturing specific biomarkers. Biomarkers can be used in clinical practice to identify risk for or diagnose a disease, stratify patients, assess disease severity or progression, predict prognosis, or guide treatment. In drug development biomarkers may be used to help determine how a drug works in the body, to determine a biologically effective dose of a drug, to help assess whether a drug is safe or effective, and to help identify patients most likely to respond to a treatment, or are least likely to suffer an adverse event when treated with a drug. Biomarkers can sometimes be used as part of the approval process for a drug or treatment, to inform regulatory decision-making.

Biomarkers or EVs for diagnostic use may be labeled with probes suitable for detection by various imaging methods. Methods for detection of probes include, but are not limited to, fluorescence, light, confocal and electron microscopy; magnetic resonance imaging and spectroscopy; fluoroscopy, computed tomography and positron emission tomography. Suitable probes include, but are not limited to, fluorescein, rhodamine, eosin and other fluorophores, radioisotopes, gold, gadolinium and other lanthanides, paramagnetic iron, fluorine-18 and other positron-emitting radionuclides. Additionally, probes may be bi- or multi-functional and be detectable by more than one of the methods listed. These antibodies may be directly or indirectly labeled with said probes. Attachment of probes to the biomarker or EVs includes covalent attachment of the probe, incorporation of the probe into the biomarker or EVs, and the covalent attachment of a chelating compound for binding of probe, amongst others well recognized in the art. For immunohistochemistry, the disease tissue sample may be fresh or frozen or may be embedded in paraffin and fixed with a preservative such as formalin. The fixed or embedded section contains the sample are contacted with a labeled primary antibody and secondary antibody, wherein the antibody is used to detect the expression of the proteins in situ.

Many methods routinely practiced in the art can be readily employed to amplify biomarkers obtained from a subject. These include assay formats such as protein PCR and ELISAs. Both local and systemic protein and peptide biomarkers can be assayed using the microneedle array devices of the invention. Other methods suitable for this purpose include analytic biochemical methods such as electrophoresis, capillary electrophoresis, high performance liquid chromatography (HPLC), thin layer chromatography (TLC), hyperdiffusion chromatography, mass spectroscopy and the like, or various other immunological methods such as fluid or gel precipitin reactions, immunodiffusion (single or double), immunohistochemistry, affinity chromatography, immunoelectrophoresis, radioimmunoassay (RIA), immunofluorescent assays, Western blotting, dipstick, and the like. For a general review of immunoassays, see also Methods in Cell Biology Volume 37: Antibodies in Cell Biology, Asai, ed. Academic Press, Inc. New York (1993); Basic and Clinical Immunology 7th Edition, Stites & Ten, eds. (1991); IMMUNOASSAYS FOR THE 80s. Voller, A. et al (editors), Baltimore: University Park Press (1981); Maggio, et al, ENZYME-IMMUNOASSAY, Boca Raton: CRC Press pp 172-176 (1980) and Tijssen, Laboratory Techniques in Biochemistry and Molecular Biology: Practice and Theory of Immunoassays, vol 15, Elsevier 1985. Reagents for performing these assays with respect to any specific protein or peptide biomarker (e.g., antibodies) can be readily obtained from commercial sources or generated by standard and routinely practiced techniques (e.g., hybridoma technology for producing monoclonal antibodies).

Binding interactions between a probe and a biomarker can be detected using a secondary detection reagent, such as a secondary antibody. For example, a “sandwich ELISA” can be used to detect binding of a biomarker to an antibody probe. The binding of a biomarker to a probe can be detected using a detection antibody that is specific to a different epitope of the biomarker. The antibody used in the detection can be of a different isotype than the antibody used as a probe, for example, an antibody probe can be an IgG antibody (including any of the subtypes, such as IgG1, IgG2, IgG3 and IgG4), and a secondary antibody can be of the IgA, IgM). The detection antibody can be, for example conjugated to a detectable label, such as a fluorescent moiety or a radioactive label. An antibody based method of detection, such as an enzyme-linked immunosorbent assay (ELISA) method can be used to detect a binding of a probe to a biomarker.

Protein and peptide biomarkers can also be assayed using protein PCR techniques. For example, one assay suitable for detecting the biomarkers in these applications is the PCR-ELISA protocol. The assay employs a standard immunoassay procedure. A capture antibody is attached to the surface of the microneedles of the devices. Instead of using a reporter enzyme for development of analytical signal, the secondary antibody is fused to a single stranded oligonucleotide that can then be amplified using PCR. After insertion and capture of the biomarkers, the presence of the biomarkers can be detected by addition of the oligonucleotide-labeled secondary antibody and subsequent PCR analysis of the conjugated tag.

In an embodiment, labeled probe may comprise a label (e.g., fluorophore, radioisotope, etc.) that can emit an optical signal. A fluorescent moiety can be a fluorescent protein, such as a green fluorescent protein (GFP), read fluorescent protein (RFP), yellow fluorescent protein (YFP) or variations thereof. In some cases, the labeled probe comprises a label with an optical signal that increases or decreases when the probe is bound to its target.

In an embodiment, the methods of the disclosure can further comprise detecting one or more biomarkers from a reference tissue obtained from the subject. For example, the method may comprise detecting biomarkers from a sample tissue and a reference tissue. The reference tissue can be a benign tissue. In some cases, the reference tissue is tissue from the same organ or region as the sample tissue. In some cases, the sample tissue comprises tissue suspected to have a disease or disorder (e.g., malignancy) and the reference tissue comprises tissue of the same organ that is known to not have the disease or disorder. In some cases, the detected levels of the biomarker in the sample tissue can be further compared to the detected levels of the biomarker in the reference tissue, as detailed in U.S. Pat. No. 9,540,684B2, which is incorporated by reference in its entirety.

Isolation Method

Interstitial fluid is a complex biofluid that surrounds cells within a given tissue. It contains various all major biological macromolecules (proteins, lipids, nucleic acids, and carbohydrates) that can be bound or unbound (membranous or non-membranous) to a particle. A category of bound particles are extracellular vesicles. Extracellular vesicles contain or express biological motifs within or on their surface. For tissue interstitial fluid extracellular vesicle diagnostic purposes, it is essential to refine/purify the non-bound interstitial solute and concentrate the bound extracellular vesicle material when harvesting and collecting tissue resident interstitial fluid EVs.

Current methods of purifying EVs involve high speed ultracentrifugation, precipitation, immunoprecipitation, or molecular sieve filtration. These methodologies have drawbacks since these methods may leave EVs permanently aggregated via precipitation, EVs may be damaged or destroyed under the shear stress and back pressure forces placed upon EVs placed in a molecular filtrate, and technical expertise is required to operate an ultracentrifuge or perform immunoprecipitation. The problem we are addressing is the separation of the IF EVs from the soluble macromolecules without damaging, precipitating, nor binding the EVs to a solid phase to retain their functionality for potential downstream analysis.

To achieve this problem, we have developed a methodology to take the harvested, raw tissue interstitial fluid EVs and apply them to a column filtration system. The column system contains an upper chamber where the raw IF EVs are applied. Within this upper chamber there is a slight to negatively charged matrix which filters molecular constituents by size. With a standard benchtop centrifuge, for 10 minutes at a low speed (12,000×g) the raw IF soluble macromolecules are transferred to the reservoir chamber below the upper chamber. The slow spin speed in combination with the negatively charged matrix, repels, or retains the EV portion of the raw IF solution within the upper chamber. Moreover, a larger pore size was found to be unexpectedly efficient at concentrating the EV portion of the raw IF solution while removing the bulk soluble non-EV portion of the raw IF into the reservoir. Thus, this method in comparison with others does not utilize precipitation agents, such as polyethylene glycol (PEG), and the mild charge distribution of the filter matrix retains the EVs within the upper chamber to ultimately retain their structure.

Example embodiments of the negatively charged matrix filter will include polymer derived biomaterials that are or can be modified to have a slight negative surface charge intended to repel/retain the negatively charged EVs and permit the various macromolecules of which are positively charged to transmit into the reservoir. An example matrix material is Polyether sulfone (PES). A common filter matrix used in the field currently is regenerated cellulose. This matrix material is ineffective at retaining the EVs and separating the EV portion from the bulk IF solute because regenerated cellulose has a slight positive charge and binds to free proteins more readily.

Another embodiment is the pore size of the matrix. A relatively large size pore of the matrix prevents back pressure and shear stress during centrifugation while the soluble macromolecules (100-1000 times smaller than EVs) enter the reservoir. The pore size prevents EV portion membrane destruction, and the charge of the matrix repels a higher proportion of EVs from entering the porous matrix as well.

In an embodiment, in vivo sampling can be performed in addition to the ex-vivo methods described in the current disclosure application.

Another embodiment or methodology to isolate tissue interstitial fluid would be an in vivo cytopathologist guided piercing of the skin into the tissue or tumor of interest wherein the isolated interstitial fluid would contain the EV portion within a probe/needle. Thus, the collected resident tissue IF EV would be collected and purified for EV content which could be utilized to measure real-time molecular or cellular information such as mitochondrial health, genetic tumor cell changes, receptor amplification, cytokine production, and cell-type specific EV markers (i.e. immune vs stromal vs tumor receptors).

The in vivo sampling of the tumor interstitial space using a probe, trocar, or needle which would evacuate by suction or pressure differentials. The probe could be flat or cylindrical, contain multiple holes on the side or at the point of insertion. The probe could be curved. The probe could have a cutting portion at the tip to pierce the tissue and contain peripheral ports for maximum extraction of interstitial fluid volume through increased surface area. The probe could have multiple piercing cylinders which would create a negative space within the tissue to collect or pool the tissue interstitial fluid for collection. The probe surface could be star shaped.

This methodology is non-invasive. This methodology is like fine needle aspiration utilized by doctors to diagnose cell state for lymphoma diagnosis. However, these methods do not collect or throw away the resident tumor interstitial fluid which contains extracellular vesicles which relate to the internal cellular or molecular states for a given tissue. This new methodology could be applied to neoadjuvant therapy because the doctor could non-invasively monitor therapeutic treatment without the need for surgery or tumor resection. This methodology could be used to monitor immunotherapy efficacy in humans or animals in-vivo.

In an embodiment, the collection of tissue resident IF EVs, can be further isolated and concentrated from the bulk IF solute in a non-destructive manner through a relatively high porosity filtration matrix that has a slight negative charge to repel and retain the EV within the EV filter retentate to achieve gentle and high throughput of the separation method with high efficiency.

In an embodiment, a means of separating the EVs from the bulk soluble molecular content within the tissue interstitial fluid by low speed centrifugation (12,000×g) including an upper chamber to receive the raw interstitial fluid and a separation matrix that is slightly negatively charged with a large porosity to minimize back pressure stress that does not break down the EV membranes such that a majority of the EVs are retained within the upper chamber and the soluble bulk IF fluid macromolecules are transmitted into the lower chamber reservoir.

In an embodiment, a pore size of the matrix should be larger than 5 nanometers, such as 10 nanometers, 50 nanometers, 100 nanometers, 200 nanometers, 500 nanometers or more.

In an embodiment, filter matrix should be at a neutral (pH 7) pH with a Zeta potential between −60 mV to −20 mV and have a surface contact wetting angle less than 50 degrees at the inlet side (upper chamber) of the device such that it achieves a 10 fold retention of the EV portion of the raw IF within the inlet (upper chamber) compared to the outlet (reservoir) fluid where the fluid is raw interstitial fluid containing EVs.

In an embodiment, the geometry and the placement of the pores and cutting portion of the probe should be designed to maximize the internal volume collection of the IF sampled. We have performed a single needle insertion and have had success with a conventional needle. Moreover, the extraction pressure “suction” should be greater than 10 mmHg and generally in the range between 10-100 mmHg, such as 20 mmHg, 30 mmHg, 40 mmHg, 50 mmHg, 60 mmHg, 70 mmHg, 80 mmHg, 90 mmHg.

In an embodiment, probe should have an outer diameter maximum of 3 mm to 0.9 mm.

The holes to collect the IF be in about 0.1 mm or proportional to the diameter such that the probe maintains structural integrity to penetrate tissue, such as 50 microns, 57 microns, 0.2 mm, 0.3 mm, 0.4 mm, 0.5 mm or more.

In an embodiment, a method comprising: selecting a living tissue of a human or an animal subject, applying a negative pressure on the living tissue; and harvesting extracellular vesicles (EVs) from interstitial fluid (IF) of the tissue, wherein the EVs are in a size in a range of about 30 nm-5000 nm, wherein the method is configured to maintain integrity of the living tissue for subsequent histopathological examination.

In an embodiment, piercing the living tissue using a needle to collect the IF from the living tissue, wherein the needle comprises a hollow needle.

In an embodiment, the needle is configured to apply an interstitial pressure. Needle can be inserted between cells. Needle can cause a negative space for interstitial fluid containing EVs to pool. The interstitial fluid pressure (IFP) or interstitial pressure of normal tissues is actively regulated through interactions between stromal cells and the extracellular-matrix molecules. The interstitial pressure usually varies depending on type of the tissue. In a tumor tissue, IFP is usually higher than a normal tissue, for example it could be 2-42 mmHg, 1-14 mmHg, 5-68 mmHg, 4-21 mmHg, 20-56 mmHg, 2-26 mmHg, 13-45 mmHg, 2-20 mmHg, 2-22 mmHg.

In an embodiment, the needle is configured to apply the negative pressure in a range of in a range between 10 mmHg to about 100 mmHg.

In an embodiment, a portion of periphery of the needle comprises one or more holes having a dimension less than a cell and configured to allow entry of EVs present in a space inside the needle.

In an embodiment, dimension of one or more holes is in a range of about 3 microns to 8 microns, such as less than 4 micron, 5 micron, 6 micron or less.

In an embodiment, a shape of the needle is either straight or bent at an angle to provide a curved shape. The curved shape comprises quarter circle, ⅜ circle, ⅝ circle or a compound circle or any shape known to a person skilled in the art, depending on type of the tissue to be pierced.

In an embodiment, a portion of periphery of the needle comprises a sheath having first end towards a bevel of the needle and a second end opposite to the first end, wherein the sheath is movable and configured to cover one or more holes. In an embodiment, bevel of the needle could be blunt shaped, star-shaped, oval shaped or any shape known to a person skilled in the art.

In an embodiment, movement of the first end of the sheath is synchronized with movement of the needle inside the living tissue to allow exposure of one or more holes so that EVs enter inside a space present in the needle.

In an embodiment, a portion of the needle comprises one or more prongs, wherein the one or more prongs is configured to cover one or more holes, wherein the needle is configured to move relatively to one or more prongs to expose or close the one or more holes.

In an embodiment, length of the one or more prongs are same or different compared to the length of the needle, such as length of the prong is less or equal to the length of the needle.

In an embodiment, the IF from the living tissue is collected in a device comprising a first chamber, wherein the first chamber comprises a matrix having at least one of (a) zeta potential between about −60 mV to about −20 mV, (b) a surface contact wetting angle less than 50 degrees and (c) a pore size in a range of 5 nm and less than 5000 nm.

In an embodiment, centrifuging the device to allow passage of macromolecules comprising proteins from the first chamber to a second chamber of the device while retaining the EVs in the first chamber.

In an embodiment, a needle is connected to a syringe, which is further connected to the device.

In an embodiment, further comprising labelling the EVs harvested from the IF to investigate one or more biomarkers present on the EVs.

In an embodiment, wherein one or more biomarkers on the EVs determine (a) spatial origin of the EVs in the living tissue, (b) an information about a state of the living tissue, and/or (c) indicator of a cellular metabolic state of the living tissue.

In an embodiment, the living tissue comprises an ex-vivo tissue.

In an embodiment, further comprising forming a section of the ex-vivo tissue, harvesting EVs on a solid phase comprising an affinity capture surface, and labelling the EVs.

In an embodiment, the section is formed using a microtome or a vibratome.

In an embodiment, wherein the ex-vivo tissue is placed on a matrix comprising a glass wool and subjected to a negative pressure to collect the IF in a chamber.

In an embodiment, about 20 microliters to about 200 microliters of IF is collected per cubic cm of the tissue sample. In an embodiment, IF could be extracted more than 200 microliters per cubic cm of the tissue such as 250, 300 microliters or more.

In an embodiment, wherein one or more biomarkers comprises CD9, CD63 or CD81.

In an embodiment, comprising fractioning the EVs harvested from the IF, wherein fractionation of the EVs is configured to separate the EVs into one or more subclasses comprising exosomes, endosomes or multi-vesicular bodies (MVB).

In an embodiment, method provides information of the cell of origin wherein the resident EVs within the interstitial fluid become modified depending on their cell of origin. This method would be subject to a test that reports the spatial origins of the EVs within the tissue. The spatial and cellular origin of the EVs in vivo can be determined by examining individual EVs within the population.

### Example 1: GFP-EVs can be Harvested Directly from GFP-Labelled 4T1 Tumour Tissue

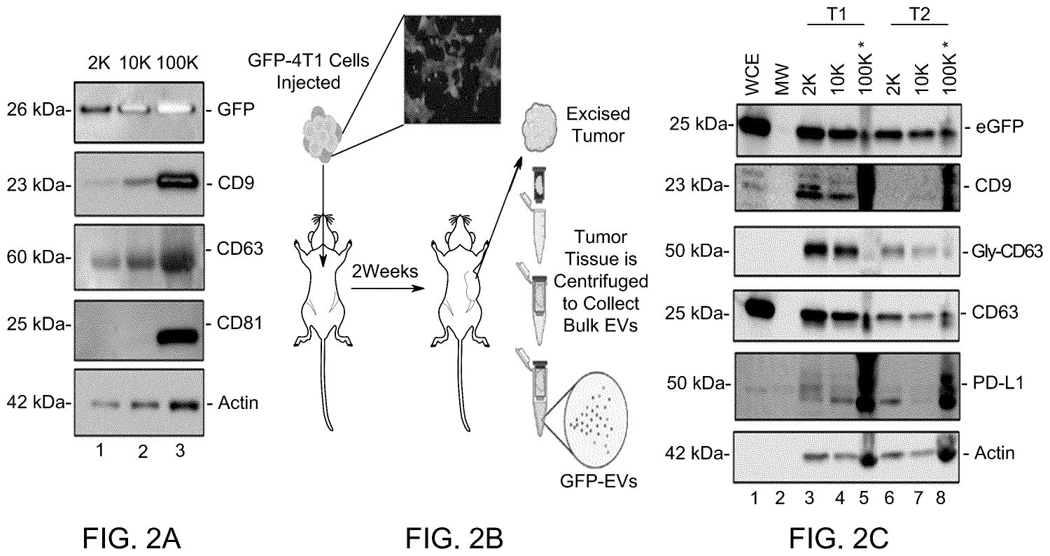

In an embodiment, we developed a method that directly harvests the resident EVs released into the IF surrounding the tumour tissue of the 4T1 syngeneic implantable tumour model (FIG. 1). To confirm the EVs collected from the fresh tissue IF were derived from tumour cells, we utilized a GFP-expressing 4T1 cell line. eGFP 4T1 cell culture-derived EVs (in vitro EVs) express GFP along with common EV tetraspanin markers, such as CD63, CD9, and CD81 (FIG. 2a). The GFP brightly stains the full cell body (FIG. 2b) and is exported within and density subpopulations of EVs in vitro (FIG. 2a). We injected 106 eGFP-4T1 cells into the mammary fat pads (orthotopic) of the syngeneic immunocompetent BALB/c mouse model. After 4 weeks, tumours 1-2 cm were excised and immediately placed into a glass wool-packed spin column (FIG. 2b).

In an embodiment, a low-speed centrifugal spin harvested the EVs from the IF gently, so the tissue sample remained histologically intact (FIG. S1), with no perturbation in nuclear fine chromatic structure, cell morphology or size. A board-certified pathologist reviewed the post-EV extraction tissue compared to the non-EV extracted tissue. The 4T1 murine orthotopic tumours are highly cellular, with a high proportion of solid sheets of carcinoma cells (Pulaski & Ostrand-Rosenberg, 2000). The size and shape of the carcinoma cell body is unaltered. Furthermore, the fine nuclear chromatin, nuclear membrane and nucleoli, as well as all other morphologic elements are identical for post IF harvesting tumour tissue as compared to freshly excised intact tumours.

In an embodiment, ex-vivo harvesting of resident IF and the EVs contained therein can be done in a few minutes. This includes the short time delay for low-speed centrifugation that harvests approximately 100 to 150 microliters of IF per cubic cm of tumour tissue. This time delay to centrifuge the tumour is one tenth, or less, of the earliest time to observe reactive phosphoprotein changes in ex-vivo tumour tissue pro-survival, hypoxia, and apoptosis pathways (Espina et al., 2008). The in vivo IF returned EVs with consistent markers for EV related tetraspanins (FIG. 2c).

Tumour 1 (T1) is an example of a larger tumour 1.5 cm which displayed high levels of tetraspanin markers for each EV fraction; however, tumour 2 (T2), an example of a superficial, smaller tumour, returned similar results, except that CD9 was only present in the 100K* population.

Thus, our IF-derived EV harvesting method was reliable, did not harm the tissue viability or histology, and returned enough EV sample after enrichment for subsequent orthogonal proteomic analysis.

### Example 2: Interstitial Fluid is a Rich Source of EVs Providing New Insights into Functional Mechanisms

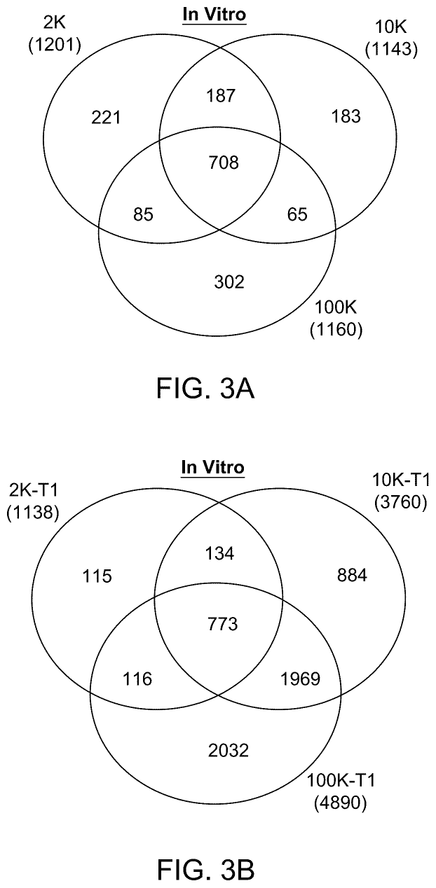

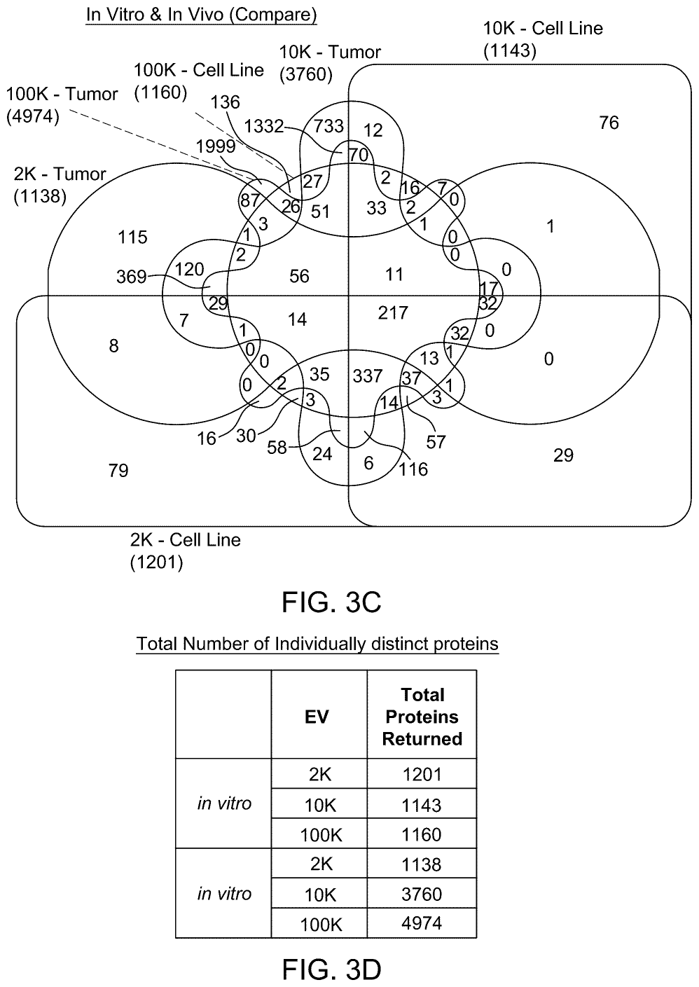

In an embodiment, we sought to understand the proteomic contents of the IF EV subpopulations. Using our high-yield mass spectrometry methodology (Magni et al., 2020), the IF EVs generated a very high content of different proteins compared to in vitro EVs (FIG. 3d).

In an embodiment, the IF EVs (in vivo EVs) returned over 3000 proteins for the 10K and 100K* EV populations alone, compared to the roughly 1000 proteins returned from the in vitro EVs (FIG. 3a,b).

In an embodiment, analysing the in vitro and in vivo EV populations all together, roughly 40% (N=1999) of the 100K* T1 EVs proteomic contents were unique to its populations. (FIG. 3c).

In an embodiment, the tumour microenvironment is comprised of a heterogeneous assortment of cell types including tumour cells, tumour activated fibroblasts, immune cells, collagenous stroma, muscle, fat, blood vessels, and lymphatics. The resident IF collected from any tissue volume will logically contain resident EVs that exist in the tissue at the time of the tissue excision, and the origin of such EVs will be a sampling of all the cell types in the microenvironment.

In an embodiment, the tumour tissue studied here is a solid tumour predominated by carcinoma cells, the IF resident fluid EVs should contain important molecular information from other cell types beyond the carcinoma cells. Furthermore, lymphocytic markers are expected within IF, as it is a conduit for lymphocytic traffic. To identify the potential additional sources of non-tumour EV proteins, beyond the predominance of carcinoma cell EVs labelled with GFP, within a given IF EV subpopulation, we reviewed our proteomic data and included the protein spectrum count for proteins within our IF EVs that are non-tumour.

In an embodiment, Specifically, we reviewed IF EV subpopulation proteomic data for common normal breast epithelial (Keratin 8 and 18), basal epithelial (Keratin 5), leukocytic (CD177, neutrophil and CD5, T-cell), and tumour-activated stromal cell markers (alpha-smooth muscle actin (α-SMA)) (Anstine and Keri, 2019; Liu et al., 2019; Menz et al., 2021). We compared protein abundance of these cell markers to common overexpressed triple negative breast cancer marker EGFR within the same IF EV subpopulation as seen in FIG. S2 (Yadav et al., 2015).

In an embodiment, relative abundance of non-tumour specific protein markers within the 100K IF EV subpopulation is significantly less than the amount of TNBC specific markers within the same IF EV subpopulation. These data demonstrate that tumour IF EVs can be a rich source of information about the functional state of all the cellular members of the tumour microenvironment. Moreover, it is important to note that tumour-activated stromal cells or cancer associated fibroblast (CAF) cells react to hypoxia by induction of autophagic flux, and can elaborate exosomes to influence the tumour microenvironment.

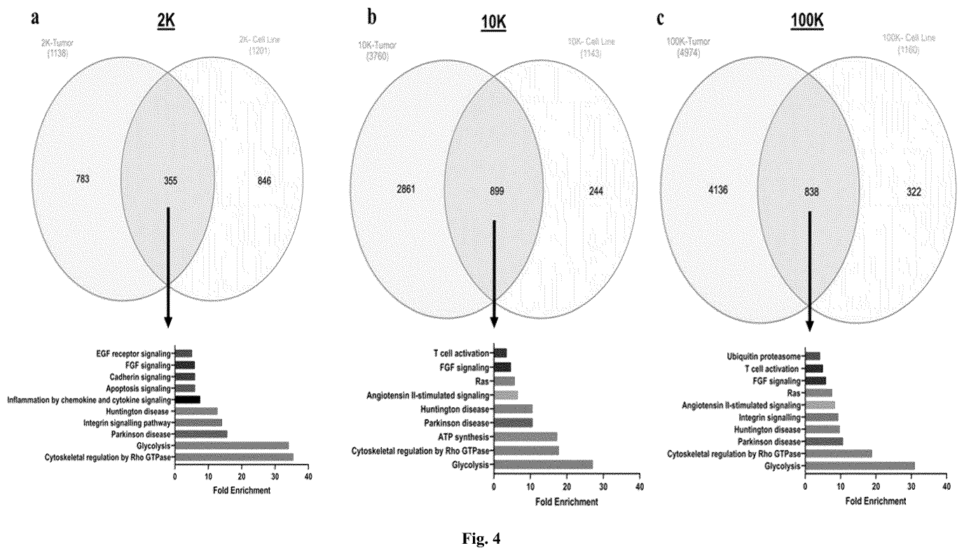

In an embodiment, we performed a head-to-head comparison by EV origin (in vitro vs. in vivo) (FIG. 4). Each EV population isolated from cell culture shared a large proportion of its total protein content with the IF EVs; however, the IF EVs have a greater, more unique molecular range of proteins returned by MS. These findings suggest that EVs derived from in vitro do recapitulate many contents found in the in vivo model (generally 25% or more), particularly for the 2K and 10K populations (FIG. 4a-b). We applied the PANTHER database pathway analysis to the proteins shared by each EV population in both cell culture-derived and IF-derived EVs. Each pathway represented in its analysis was significantly enriched (p>0.05) compared to NCBI mouse proteome. We found that the 10K and 100K EVs, regardless of origin, shared immune cell activation pathways (T-cell activation) whereas the 2K EVs shared inflammatory pathways, suggesting potential roles in immunomodulation as has been reported for EVs (FIG. 4b-c) (Chen, Zhao et al., 2018b; Groza et al., 2020; Song et al., 2019).

We found that EV subpopulations often had proteins indicative of pro-growth pathways such as fibroblast growth factor signalling, glycolysis, Rho-GTPase, and Ras family pathways suggesting a potential role of EVs in angiogenesis and other pro-growth survival pathways. Lastly, all EV subpopulations shared markers that are related to dysfunctional endocytic vesicle trafficking within the cell, which is typically coordinated by the degradative autophagy pathway (Wang et al., 2017). Overall, IF is a rich source of EVs with a large, dynamic proteomic output.

### Example 3: Utility of the 4T1 Model to Investigate Functional EV Differences for Sentinel Lymph Node (SLN) Immune Priming

The proteomic similarities between in vivo IF versus in vitro EV subpopulations raises the question as to whether the subpopulations of EVs in the 4T1 model have a differential impact on the host immune recognition process occurring in the downstream SLN. Of note, the CD81+/CD63+/CD9+ 100K* IF EVs have a high programmed death ligand 1 (PD-L1) content (FIG. 2c). It has been hypothesized that PD-L1+ EVs promote tumour growth by immune suppression through the presence of PD-L1 on their surface (Chen, 2018b; Poggio et al., 2019; Song et al., 2019).

In an embodiment, we evaluated the utility of the 4T1 model to use EV IF findings of the present study to test EV functional roles in tumour immunology. As shown in FIG. S4, we compared the in vivo immunogenicity between the 100K and the 2K EVs. This experiment was modified from a previously established immunocompetent animal model to overcome the immune suppression of a B. anthracis infection at the SLN (Popova et al., 2015; Teunis et al., 2017).

The model utilizes hydrogel nanoparticles (NPs) pre-loaded with immune chemoattractant proteins (CKs), such as CCL18/PARC an CXCL9/MIG, as a carrier to direct pre-loaded EV subpopulations. The dual action immunization deposits EVs and CK releasing NPs to the SLN subcapsular sinus with high yield. The purpose was to recruit immune cells to recognize the simultaneously injected EVs (FIG. S1a). After 48 h, 106 4T1 cancer cells/ml were injected at the same site (FIG. Sib). The 100K EV treatment in this feasibility study was associated with a reduction in tumour growth and distant metastasis (FIG. S4c-h), suggesting that pre-treatment with CK/100K-EV/NPs potentially primed the immune system to overcome PD-L1-mediated suppression.

In contrast, the 2K EV treatment dramatically induced tumour growth and distant metastasis compared to the control group (4T1 cancer cells alone) (FIG. S1c-h). One component of this functional difference could be related to the concentration of pro-angiogenic pathways (Chiba et al., 2018; Madu et al., 2020; Pircher et al., 2011) within the 2K EVs (FIG. 4a).

The MS data revealed that IF EVs contained markers for angiogenesis, specifically Annexin II and hepatocyte growth factor (Table 3—Inflammation, Angiogenesis, & Immune Activation) (Leblanc et al., 2015; Liu & Hajjar, 2016; Maji et al., 2017). The T1 tumour EV subpopulations were analysed together using Proteome Discoverer software with the NCBI Mouse proteome database. A selected group of proteins for enriched pathways (Inflammation; Angiogenesis and Immune Activation and Autophagy) were grouped. Heatmap presence visualization demonstrates which markers for the selected proteins are in which EV subfraction.

In an embodiment, to better understand the angiogenic propensity of the EVs, we applied the cell culture EVs to an in vitro angiogenesis co-culture system using hTERT immortalized mesenchymal stem cells and immortalized aortic endothelial cells and let them incubate for 6 days. After 3 days, phase contrast microscopy revealed higher levels of spheroid formation on the cells treated with 2K EVs; furthermore, after 6 days, the cells treated with the 2K EV population had not only significantly increased tubular formation, but remarkably increased cell-to-cell contacts and branching (FIG. S5a-c). We further probed these cell culture-derived EVs for vascular endothelial growth factor (VEGF), a major pro-angiogenic signalling protein, via Western Blot and found that the 2K population expressed higher levels than the 10K and 100K EV populations (FIG. S5d).

These in vivo and in vitro 4T1 data show how the model system proposed in the present study can go back and forth between in vivo and in vitro to pursue questions relevant to EV biology based on the striking molecular differences within the IF EV subpopulations.

### Example 4: Secretory Autophagy is a Major Contributor to the Repertoire of IF EVs

The proteomic data returned from the IF EV subpopulations revealed an unexpectedly high content of markers associated with autophagy. Specifically, a variety of Ras-related proteins in brain (Rab) GTPases specific for aiding in the regulation of autophagosome biogenesis, trafficking proteins, and clearance proteins specific to the degradative autophagy pathway (Table 3—Autophagy) (Morgan et al., 2019) was found. These Rab proteins were found in all EV subpopulations with increasing frequency within the 100K population.

Growing evidence has indicated that EVs can be released via secretory autophagy pathway in the form of an autophagosome (Dupont et al., 2011). The MS data returned Rab-33b, a major regulator of autophagy which has been shown to be required in the release of Hepatitis B virus capsid by secretory autophagy (Morgan et al., 2019). Additionally, the molecular trafficking protein SEC22 homolog B (SEC22b), another indicator of a secretory autophagy (New & Thomas, 2019), was found in the 100K IF EVs.

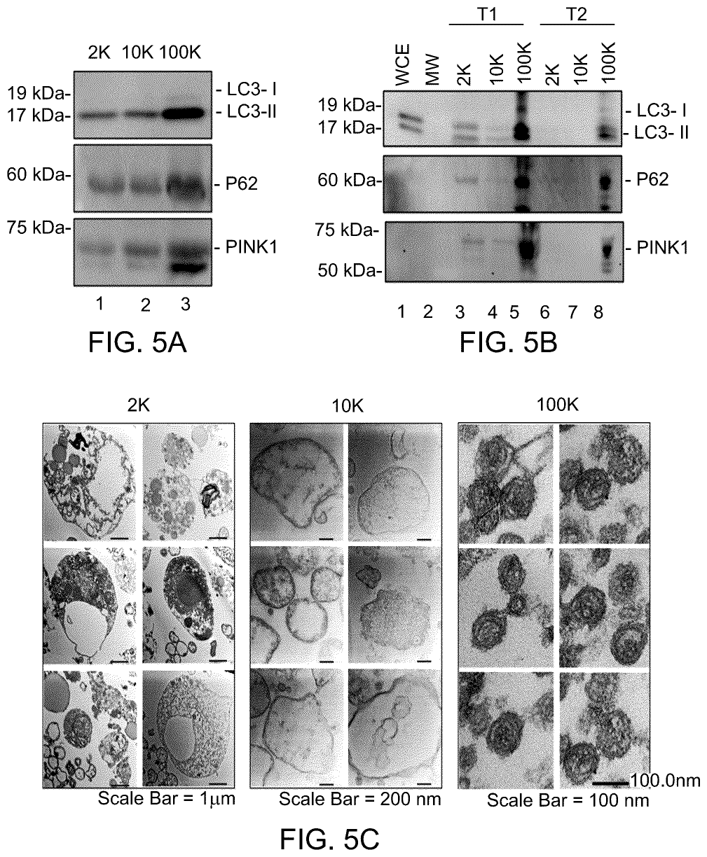

In an embodiment, we further probed these vesicles via Western Blot for key autophagosome markers such as microtubule-associated proteins 1A/1B light chain 3B (LC3) and sequestome 1/p62 (Mizushima & Yoshimori, 2007; Yoshii & Mizushima, 2017). Our in vitro EVs demonstrated increasing intensity levels of LC3-II and p62 from 2K to 100K population (FIG. 5a). The presence of LC3-II rather than LC3-I indicates a fully formed autophagosome due to the lipidation of LC3-I by phosphatidylethanolamine. The lower intensity levels seen in the 2K population of LC3-II, without the presence of LC3-I, potentially shows the initiation of LC3-II degradation rather than lack of autophagosome accumulation (Mizhushima & Yoshimori, 2007; Yoshii & Mizushima, 2017). p62 is a substrate for autophagy found within the autophagosome (Katsuragi et al., 2015). Therefore, lower intensity values after immunoblotting indicate the degradation of the contents within the autophagosome. Likewise, the IF EVs for both tumours T1 and T2 demonstrated the same pattern of LC3 and p62 partial autophagosome degradation in the 2K population and autophagosome accumulation within the 100K population (FIG. 5b). A central indicator of an autophagosome is a double membrane (Ekelinin, 2005, 2008; Yoshii & Mizushima, 2017).

In an embodiment, we imaged the in vitro EV via transmission electron microscopy (TEM) (FIG. 5c, FIG. S7). For the 2K population, we found a very heterogeneous population of vesicles with varied sizes (roughly 1-5 μm) and visible subvesicular contents within the larger vesicular body, with varied electron densities. The 2K population has many visible vesicles within a central vesicle as seen by the darker more electron dense regions, indicative of partially degraded material (Ekelinin, 2005, 2008). These visual results show that the 2K population is more likely a collection of multivesicular bodies (MVBs) that have initiated the degradative process of autophagy, colloquially known as an amphisome (Ekelinin, 2005, 2008; Patel et al., 2013; Sanchez-Wandelmer & Reggiori, 2013). The 10K population contained varied vesicular sizes ranging from 0.5-1 μm, but without as many clear subvesicular structures as the 2K (FIG. 5c). Most of the 10K bodies appear visually lucent, therefore it is unclear whether this EV is either an autophagosome, an empty vacuole, or potentially a vesicle containing further degraded material. In contrast to the larger sized EVs, the 100K population TEM exhibited a vesicular population that was highly homogenous by size (approx. 100 nm), and morphology. Each vesicle displayed an autophagosome-like clear double membrane and dense interior content (FIG. 5c, FIG. S7).

These data emphasizes that each EV contains autophagosomes within their population that are secreted rather than degraded. Moreover, the results suggest that EV subpopulations represent different members of the autophagy pathways where the 2K EV population represents an amphisome-like population and the 100K population represents fully mature, non-degraded autophagosomes.

### Example 5: A Complete Set of Parkin-Independent Mitophagy Proteins is Associated with IF EVs

In an embodiment, an unexpected finding in the IF EVs was a complete repertoire of fission-associated Parkin-independent mitophagy proteins. Specifically, the IF 100K* EVs contain several E3 ubiquitin ligases implicated in Parkin-independent mitophagy: Ariadne RBR E3 Ubiquitin Protein Ligase 1 (ARIH1); HECT, UBA And WWE Domain Containing E3 Ubiquitin Protein Ligase 1 (HUWE1) (Melino et al., 2019); and Smad ubiquitin regulatory factor 1 (SMURFI) (Villa et al., 2018) as shown in Table 4—E3 Ubiquitin ligases. The T1 IF EV subpopulations were analysed together using Proteome Discoverer software with the NCBI Mouse proteome database. The analysis revealed an unexpected presence of mitochondrial markers associated with a select pathway of mitophagy (Parkin-independent mitophagy). Specifically, the sub-pathways of E3 Ubiquitin Ligase and Fission Initiated Mitophagy were enriched in the 100K EV subtype.

In addition, the IF 100K* EVs contain mitochondrial fission markers such as dynamin-1 like protein (DRP1) and mitochondrial fission protein 1 (FIS1), both of which have been noted to regulate the cleaving of damaged mitochondria in a fission-mediated mitophagy pathway (Table 4—Fission Initiated) (De Paepe, 2012; Roberts et al., 2016; Villa et al., 2017, 2018; Yoon et al., 2003). These results represent a sequential record of the stages of Parkin-independent mitophagy.

In an embodiment, to understand the location of PINK1 full length protein or its fragments within the IF 100K EVs, from different tumors, we used well-established trypsin treatment of the EVs using a protocol for studying PINK1 cleavage product. Initial western blots demonstrated that PINK1 was associated with the EVs, including different characteristic high molecular weight, or intermediate molecular weight, fragments of PINK1. This effect was reproducible across multiple tumors, and TOM20 intensity was directly correlated with PINK1 intensity. We subsequently examined, the trypsin sensitivity of PINK1 within the 100K IF EVs. These experiments demonstrated that full length PINK1 was sensitive to trypsin degradation in a dose dependent manner, while PINK1 characteristic fragments were partially trypsin resistant.

In an embodiment, we probed the EV subpopulations for PINK1 via Western Blot. The mitophagy pathway is initiated when PINK1 can no longer be processed by the protease PARL (Presenilins-associated rhomboid-like protein) leading to PINK1's accumulation on the outer membrane of the mitochondria. PINK1 forms a dimer with (TOM20) and this contributes to sequestering of the full length form of PINK1 onto mitochondria targeted for mitophagy.

In an embodiment, For the EVs within the tumour IF, we verified that PINK1 and TOM20 were found together in the same EV subfraction (FIG. S9a) and that the levels of the two proteins were correlated, in keeping with the expected dimerization. The accumulation of full length PINK1 will initiate an E3 ubiquitin ligase to recruit the autophagy machinery, such as p62 and LC3, to enclose the organelle within an autophagosome. For both in vivo and in vitro EVs, PINK1 and TOM20 was preferentially found in the 100K population (FIG. 5a, b; FIG. S9a).

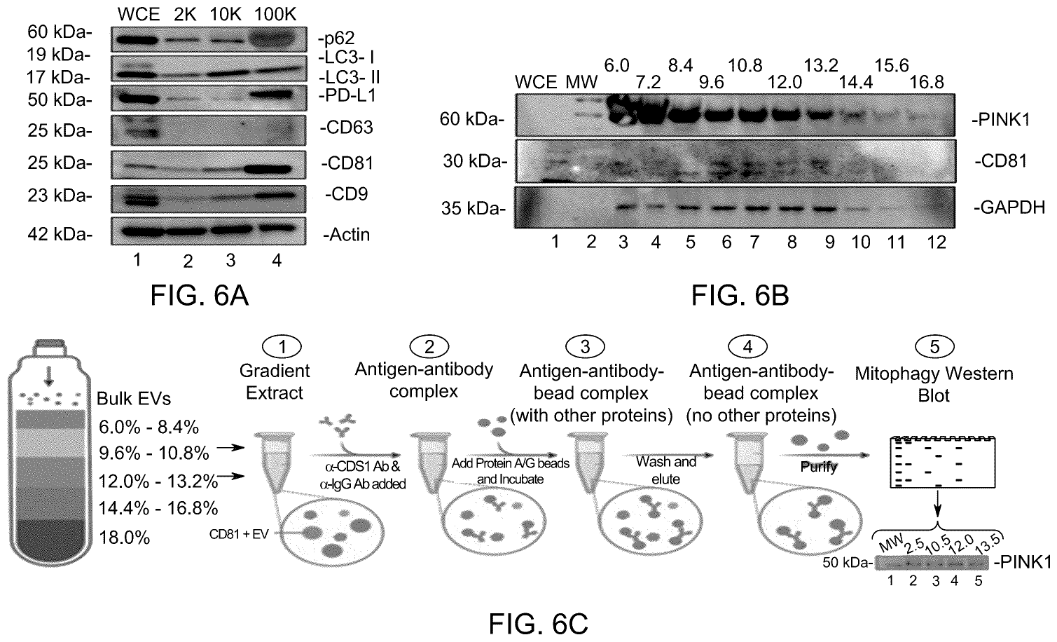

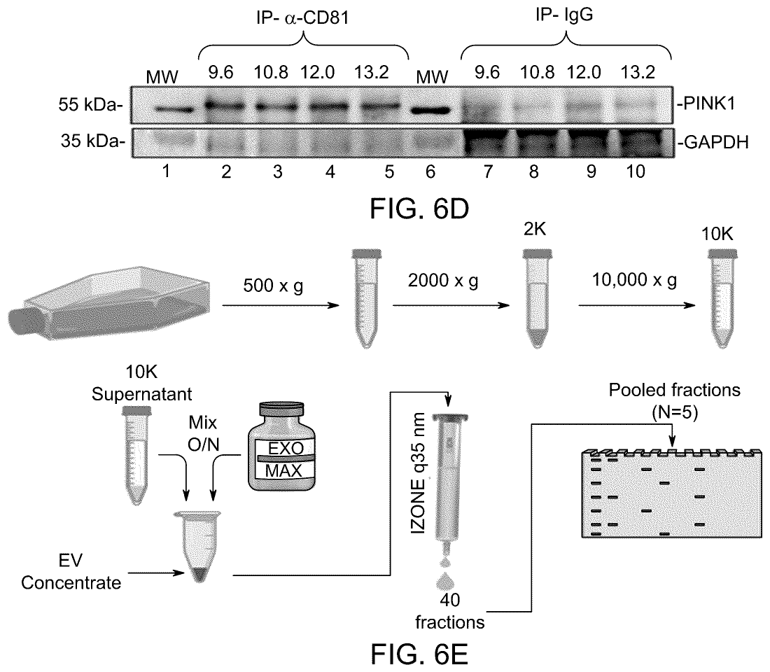

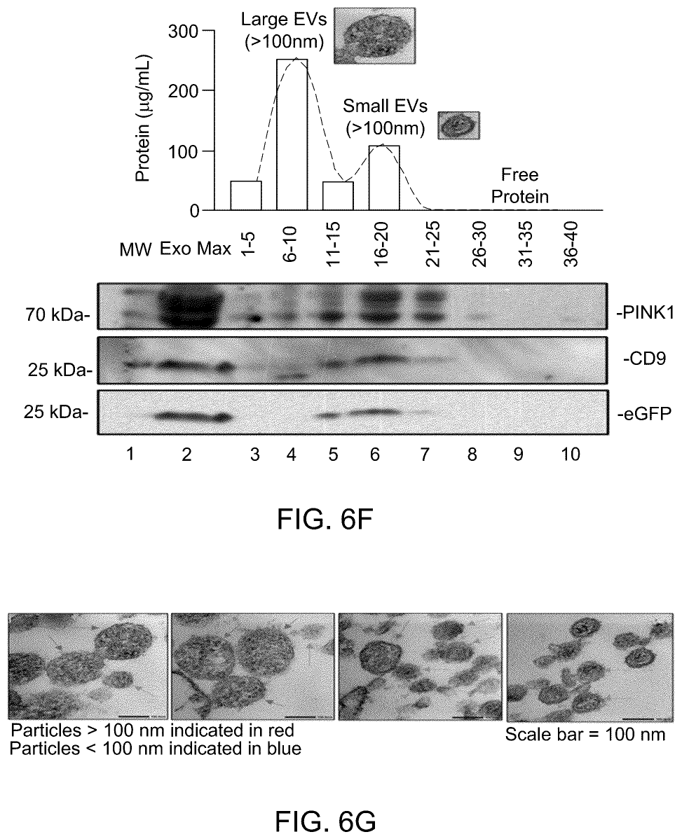

In an embodiment, biological process of EV export of Parkin-independent and fission-associated mitophagy specific proteins was not limited to one cell type. Human triple-negative breast cancer MDA-MB-231 EV subpopulations were isolated by differential ultracentrifugation and probed for tetraspanin and autophagy markers (FIG. 6a). The distribution of EV markers were similar between the eGFP-4T1 EVs and the MDA-MB-231 EVs. Similar to the mouse EVs, the 100K population of human MDA-MB-231 breast cancer cell EVs are enriched for PD-L1. The human breast cancer EVs also exhibit autophagosome properties as seen by the presence of p62 and LC3-II. The 100K population contains a large amount of p62 which would indicate a complete, undigested secreted autophagosome.

Based on these similarities, we sought to identify the density fraction that would contain PINK1. Using an iodixanol density gradient we identified that PINK1 was present at lower densities (6.0%-14.4%) (FIG. 6b). Next, we selected for the specific densities (9.6%-13.2%) that have been reported to contain the 100K differential ultracentrifugation population of “large 100K exosomes” via immunoprecipitation (IP) with anti-CD81 (FIG. 6c). The IP confirmed that the CD81+ EVs derived from the human MDA-MB-231 cell line contained the critical mitophagy marker for PINK1 (FIG. 6d).

Regarding the potential contribution of free proteins (not associated with EVs) that influence the signal of mitophagy-related PINK1 found in the 100K* pellet, we used size exclusion chromatography (Demarino et al., 2018, 2019) to assess whether the important mitochondrial proteins observed in this EV subpopulation existed in the solution phase (FIG. 6e). This chromatography method successfully separates free proteins from the EVs, as show in FIG. 6f, and enriches for two potentially distinct EV subpopulations (see example TEM images of FIG. 6g). The results indicate that PINK1, eGFP, and CD9 were associated with smaller 35-50 nm EVs (fraction 16-20, FIG. 6f), and that the fractions of free proteins were devoid of these same proteins. This enrichment and precipitation method was further validated to demonstrate a population of PINK1 EVs (Gly-CD81+, CD81+, and Alix+) are not associated with the free protein region of the column by western blotting and imaging by SEM (FIG. S10).

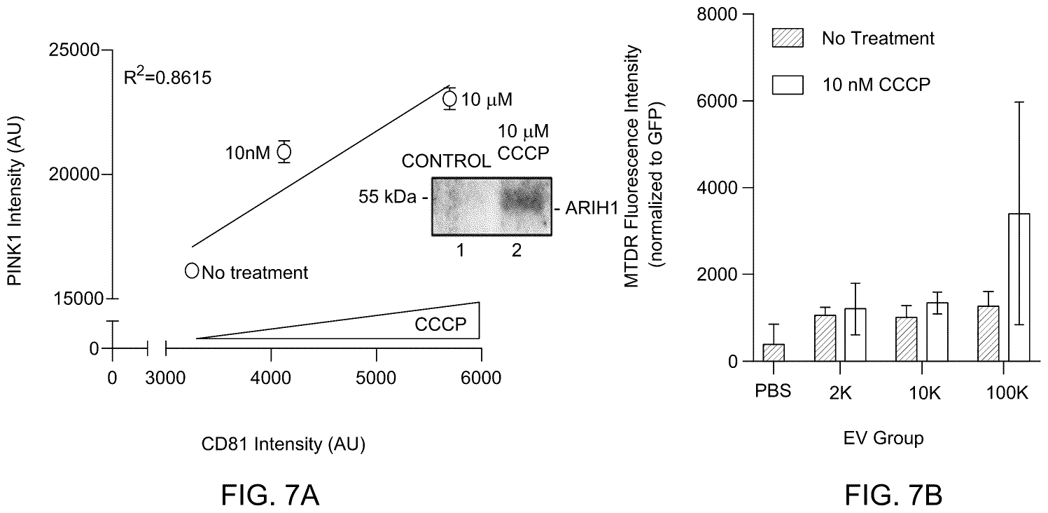

To further verify that PINK1-associated EVs contained mitochondrial components via a orthogonal methods, we performed a functional perturbation in vitro to induce the cellular release of mitophagy-associated EVs. We treated sub-confluent eGFP-4T1 cells with a mitochondrial depolarization protonophore carbonyl cyanide m-chlorophenyl hydrazone (CCCP) (Villa et al., 2017) and added a permanent fluorescent label for active mitochondrial membranes with MitoTracker Deep Red (MTDR) (Chazotte, 2011). After 48 h, the cells reached 70% confluency and were harvested by differential ultracentrifugation. We found that increasing CCCP to a cytotoxic level (10 μM CCCP) lead to increased release of CD81+/PINK1+ 100K EVs in a dose dependent manner (FIG. 7a). We verified that the treatment of CCCP induced ARIH1 presence in the 100K EVs compared to no treatment as well (FIG. 7a). The presence of MTDR was found in all EV subpopulations with increasing levels in the CCCP-treated 100K EVs (FIG. 7a).

We correlated these findings with GFP and found that regardless of treatment status, GFP was released consistently in all EV subpopulations (FIG. 7b). Additionally, MTDR found within the EVs separately confirms that active mitochondrial components can be secreted into EVs by inducing cellular mitophagy. These data provide independent evidence of transfer of mitochondrial molecules of the Parkin-independent pathway into specific EV subpopulations, particularly under metabolic stress.

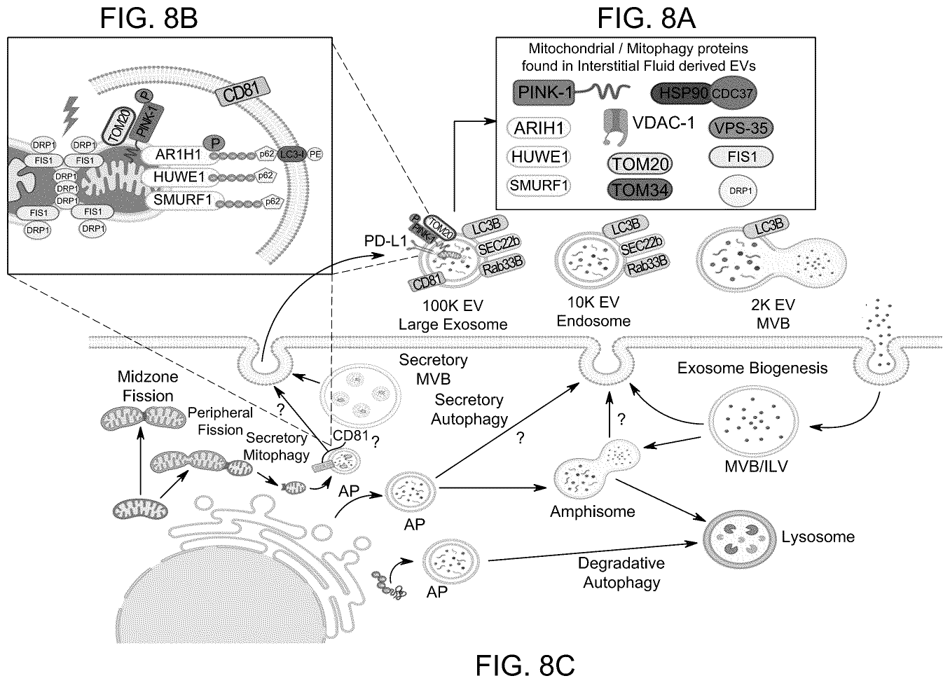

We conclude that the general biological class of mitophagy protein enriched EVs are a reflection of internal mitophagy or metabolic stress that is not limited to species (murine vs. human). This implies that a more general type of EV mechanism that contains critical machinery for mitophagy exists for the removal of damaged or excess mitochondrial components (FIG. 8).

### Example 6: Collection of EVs

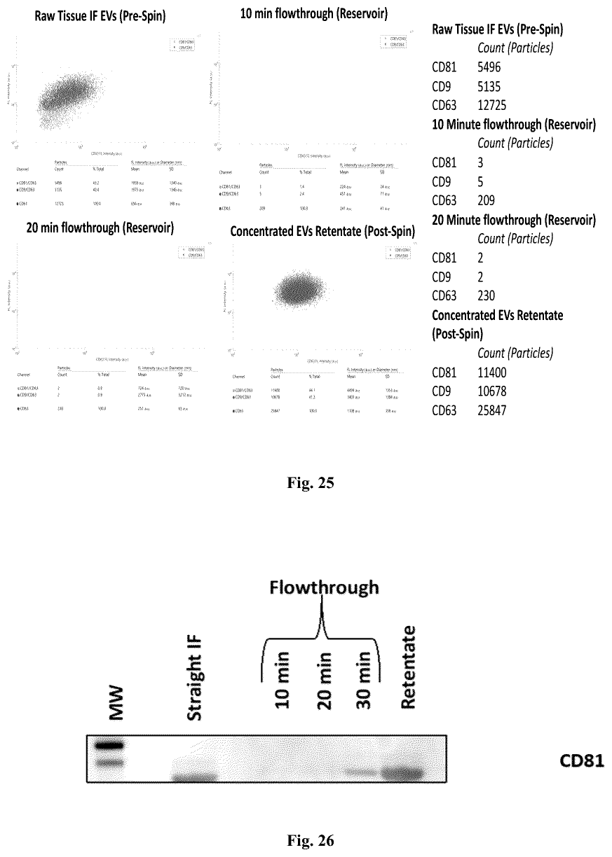

A PES matrix with a relative pore size of 10-14 nm was pre-rinsed with PBS and spun at 12,000×g for 10 minutes twice. Following matrix pre-rinsing, the bulk raw tissue IF EVs were placed into the upper chamber of PES matrix. A 10-minute spin at 12,000×g was performed. The flow through material was collected and save. Another 10-minute (20-minute total) spin at 12,000×g was performed. The flow through material was collected. The upper chamber retentate was saved as well. Approximately, 25 uL of the bulk raw IF EV, the 10 minute and 20-minute flow through material, and the retentate was deposited onto Unchained Lab's Leprechaun Luni Chips separately. The Luni Chips contain conventional EV tetraspanin marker antibodies against CD81, CD9, and CD63 on their surface. After incubation, washing, and florescent staining, the Luni Chips were analyzed with a florescent interferometer. This system counts the florescent intensity of individual EV particles and sizes each EV particle. It was found that the EV retentate had a 2-fold increase in EV particle concentration compared to bulk raw IF EV. Additionally, unexpectedly, the flowthrough material contained minimal amounts of EV particles thus demonstrating the efficacy of the PES membrane with large pore sizes retains the EV portion of the bulk IF, as shown in FIG. 25.

Like FIG. 25, the material following 10-, 20-, and 30-minute spins at 12,000×g was collected in the reservoir. The bulk IF (Straight IF) and Retentate was loaded as well. The Retentate had a larger stronger band for CD81. Moreover, it was determined that after 30 minutes, the EV portion starts to move into the reservoir. This is likely due to the shear stress and destructive forces of spinning the EVs against the matrix for 30 minutes. Therefore a 10-minute spin speed was determined to be sufficient and efficient at concentrating and purifying the EV portion of the IF without destroying the particles, as shown in FIG. 26

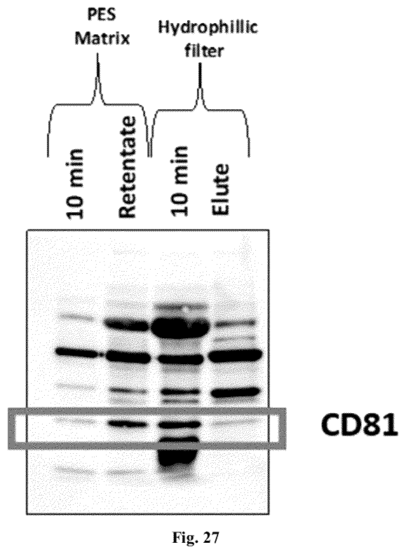

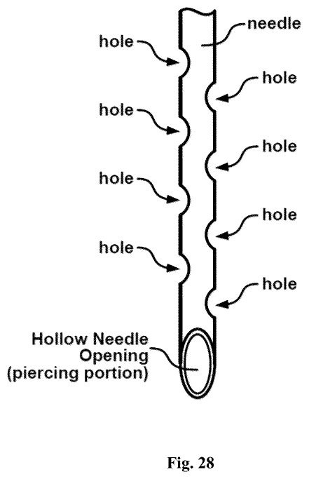

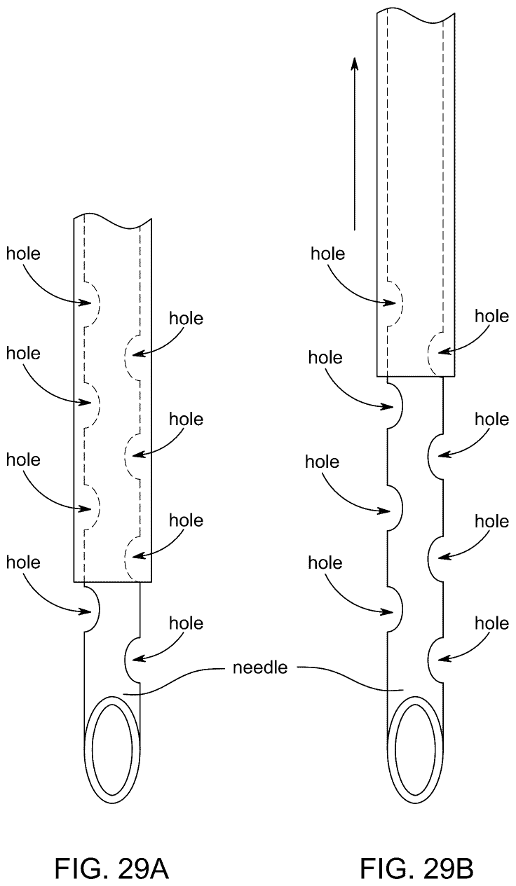

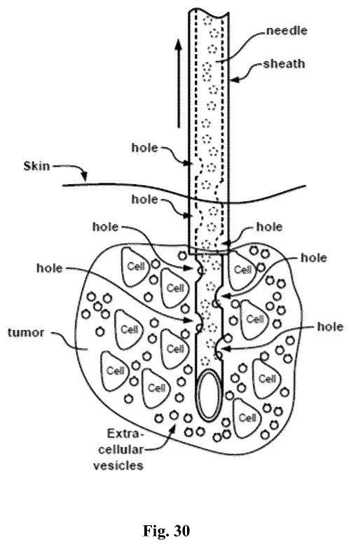

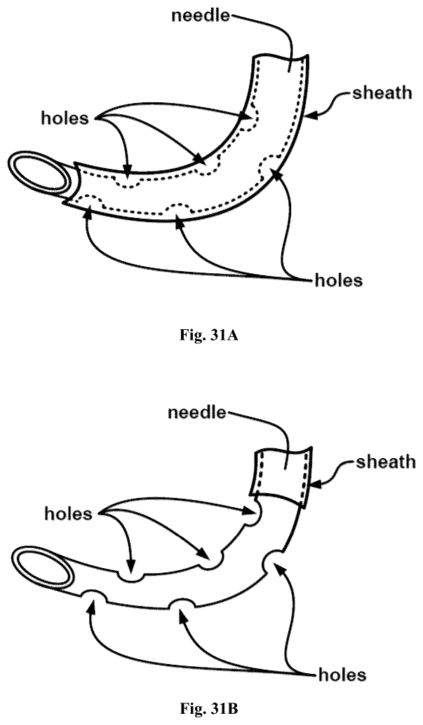

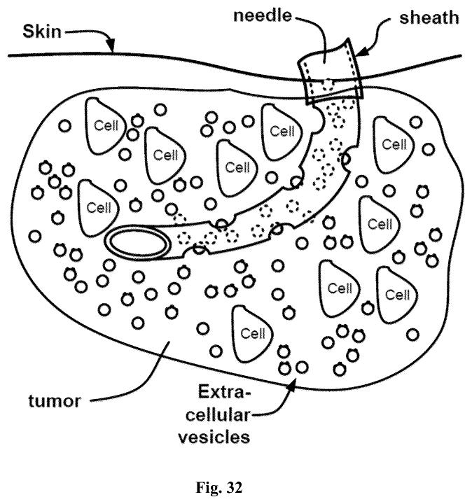

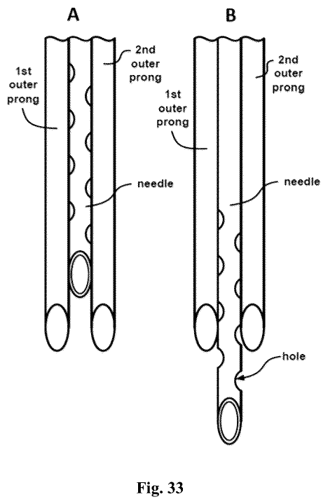

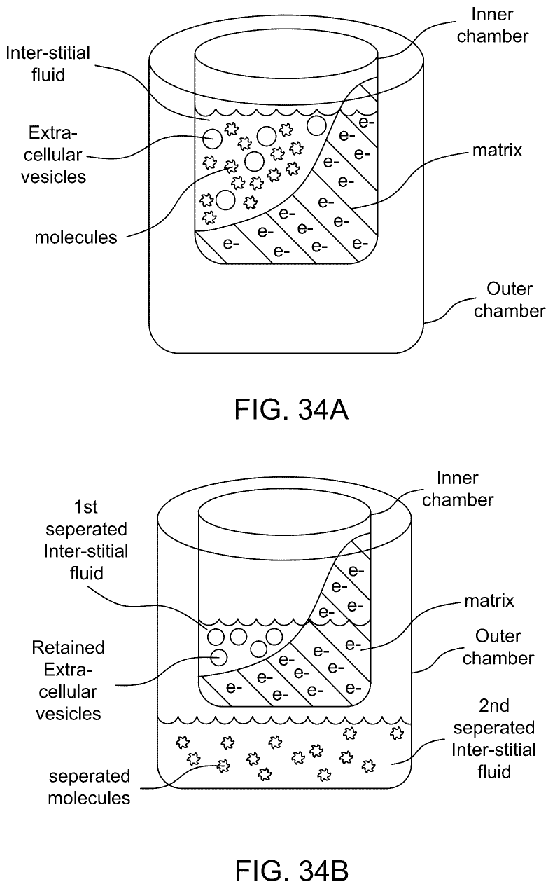

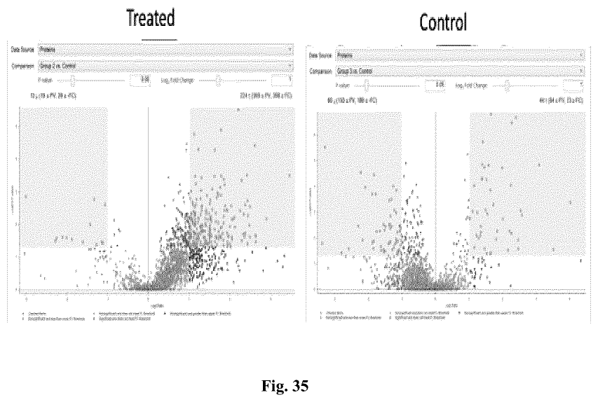

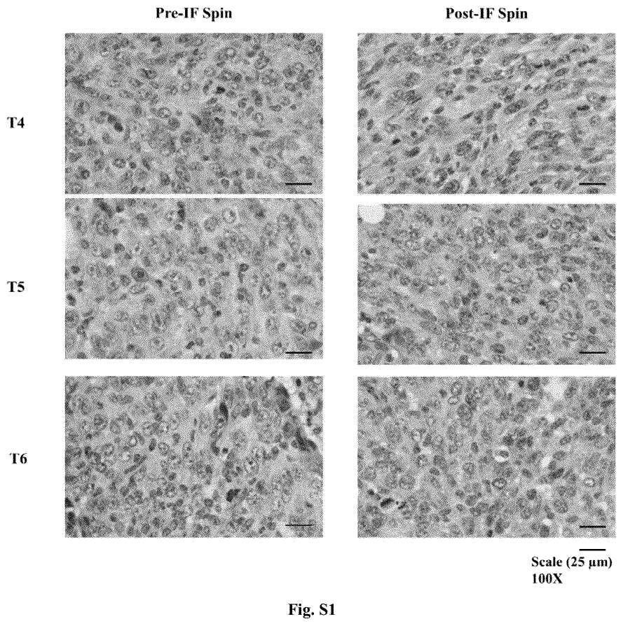

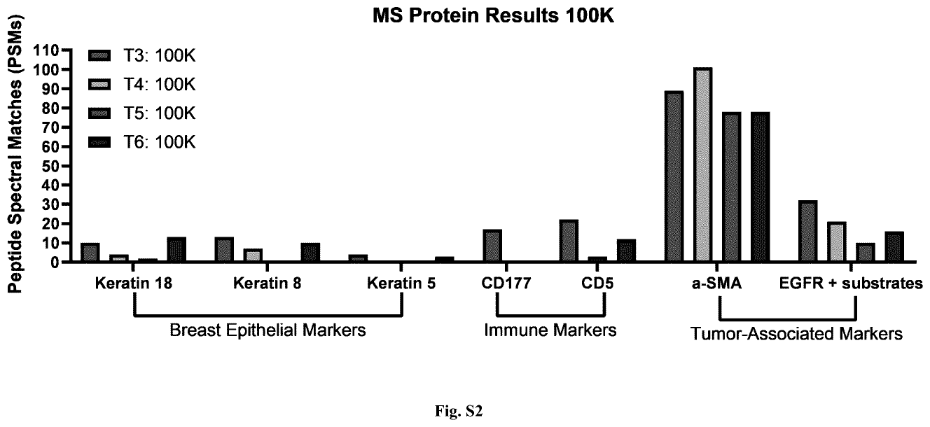

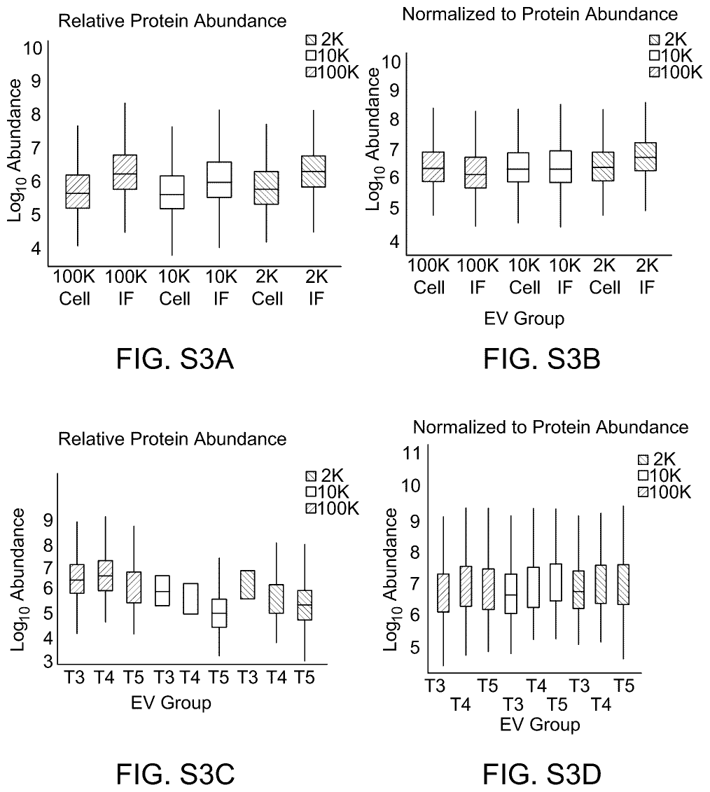

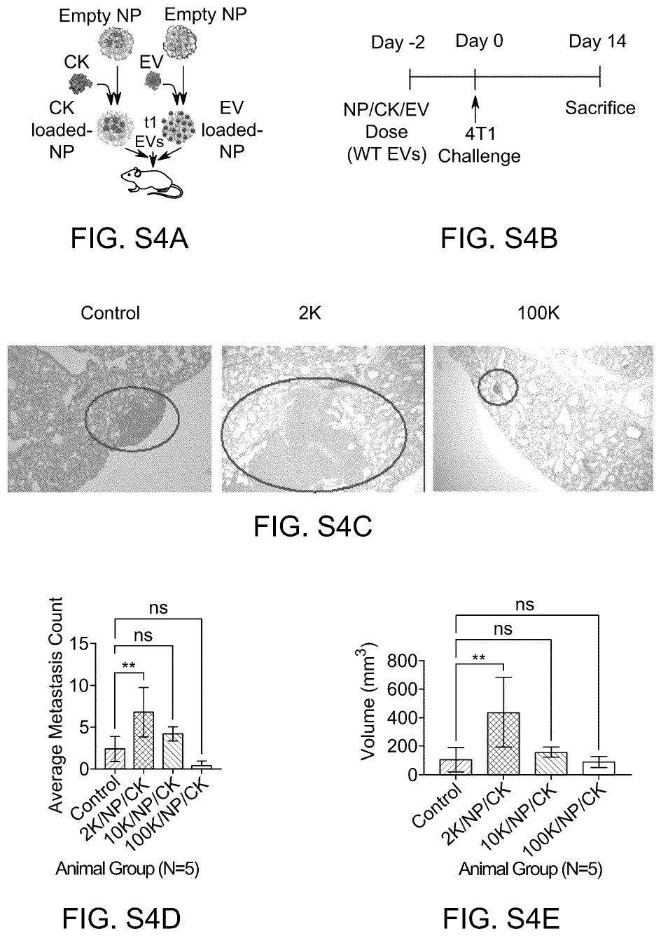

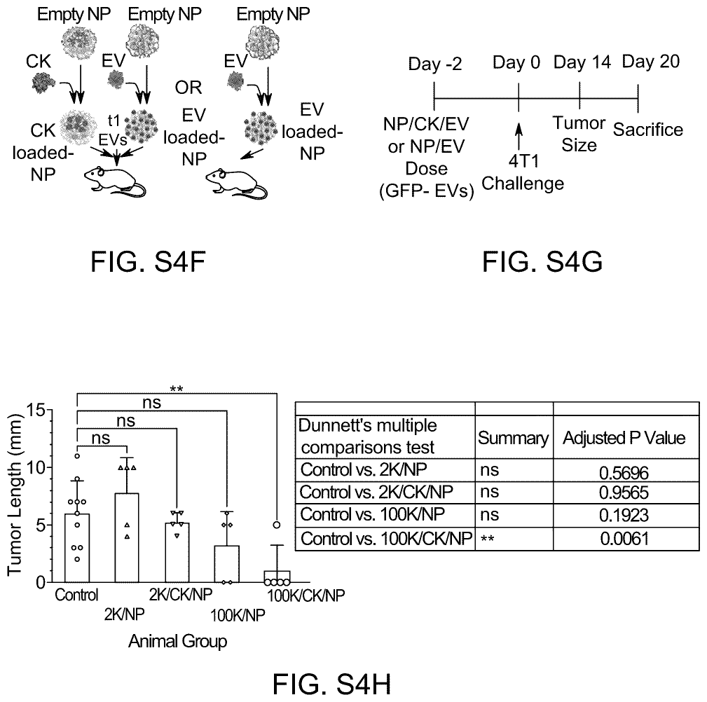

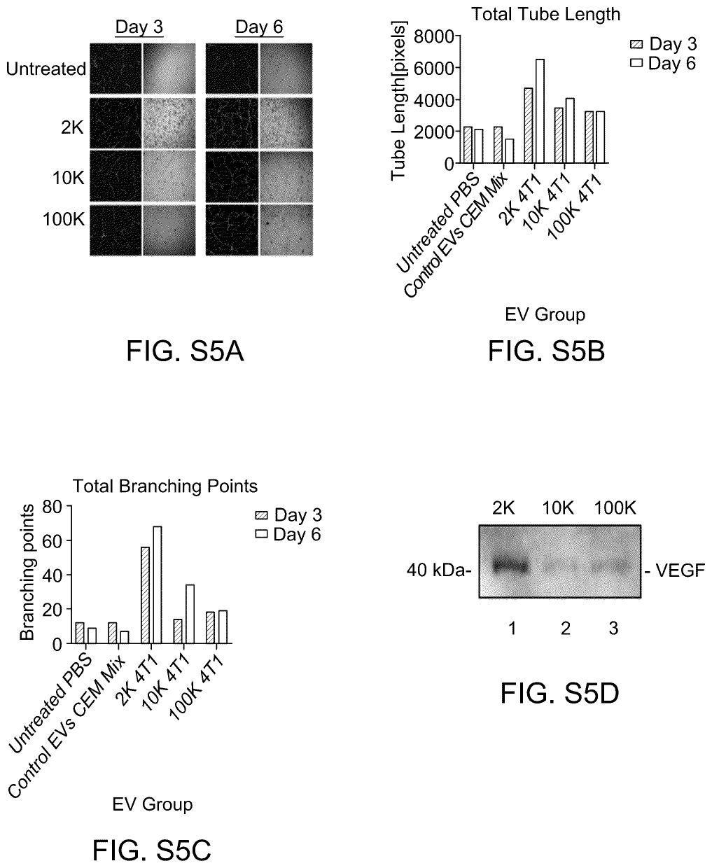

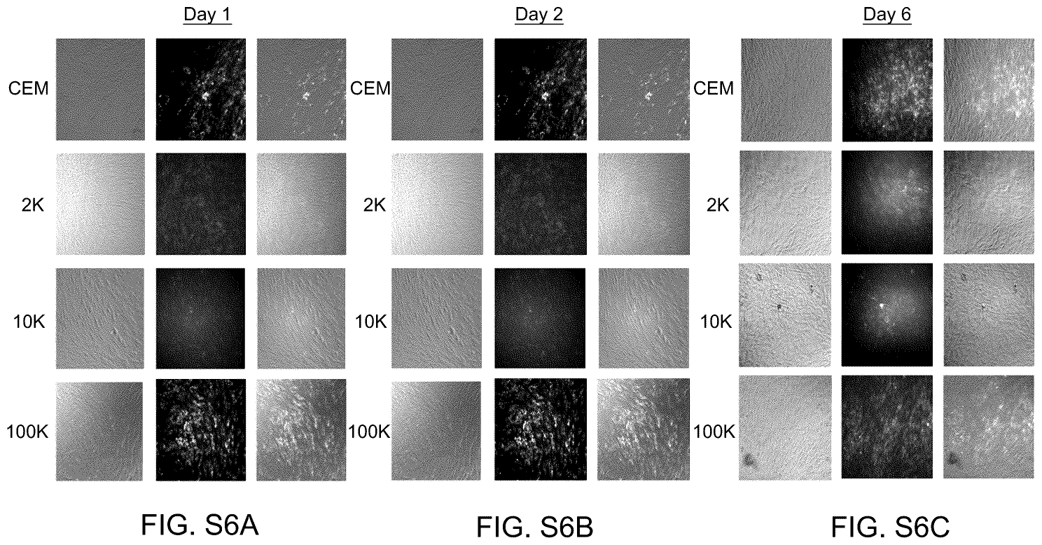

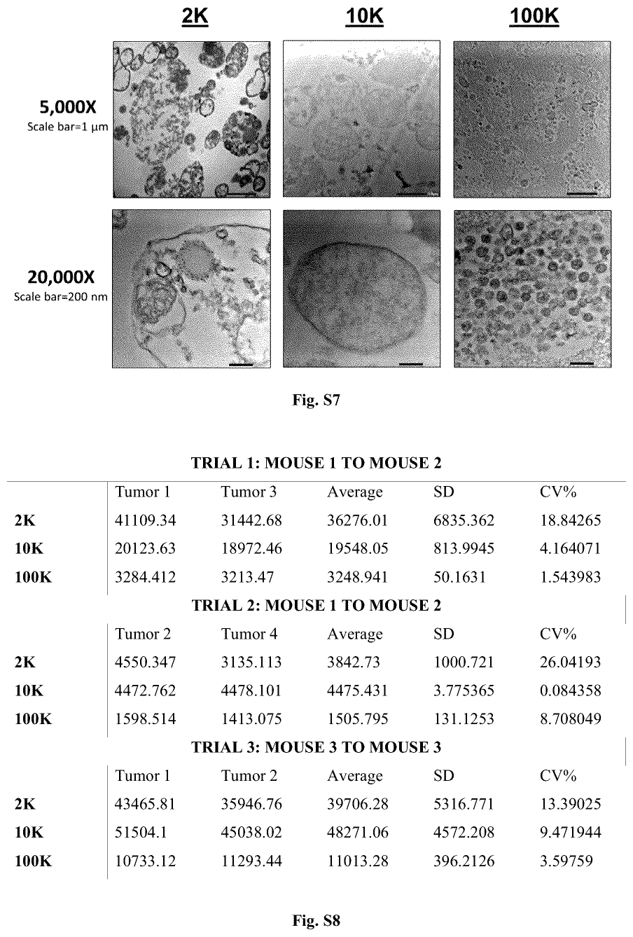

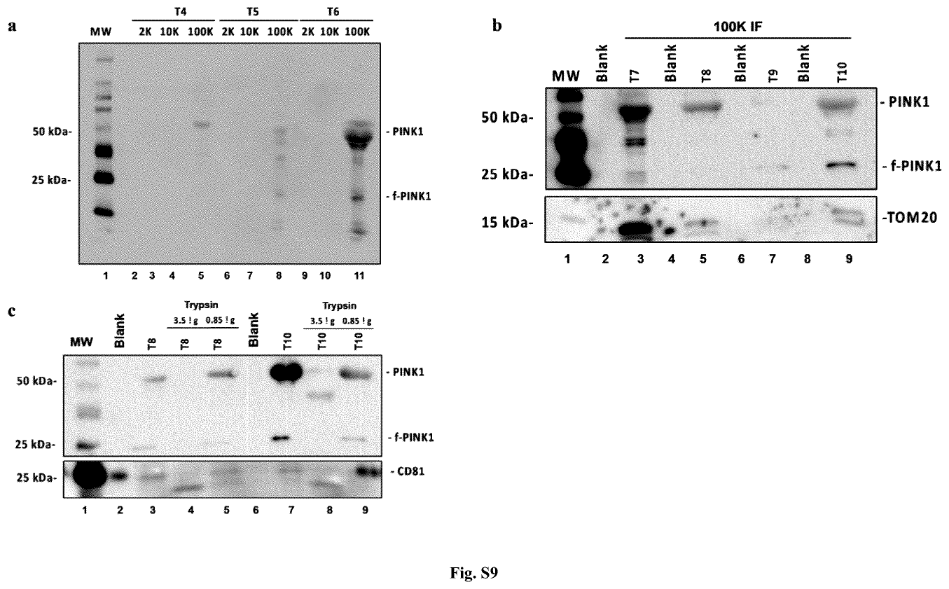

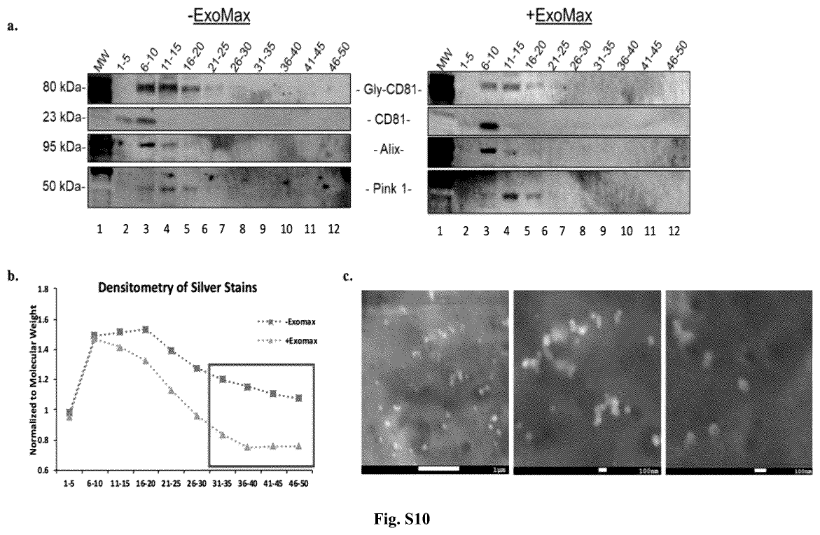

The PES matrix was compared to a hydrophilic bead-based column system. The bulk IF solution was applied to the upper chambers of each and the 10-minute flow through was collected. The upper chamber retentate for the PES membrane was collected. The hydrophilic matrix did not retain any fluid portion within the upper chamber. Therefore, an elution buffer was applied to determine how much EV portion retained in the hydrophilic bead matrix. It was found that the hydrophilic bead matrix did not retain CD81 positive EVs nor did it separate any fluid portion of the bulk raw IF. Therefore, a charged matrix with a pore barrier is required to retain the EV fluid portion of the raw IF as well as concentrate and preserve the IF EV portion, demonstrated in FIG. 27.

In an embodiment, zeta potential of the matrix is calculated by the Helmholtz-Smoluchowski equation as defined in Sabbatovskiy, K., Sobolev, V., Tsygurina, K., Kirichenko, E., & Kirichenko, K. (2022, February). In IOP Conference Series: Earth and Environmental Science (Vol. 979, No. 1, p. 012153). IOP Publishing.

### Discussion

EVs shed from cells embedded within the complex in vivo tissue microenvironment passively enter the interstitial space of the tissue where they are swept into the adjacent interstitial fluid (IF). The tissue IF samples from various tissue regions merge with the lymphatic drainage for downstream filtration and immune cell examination within the local sentinel lymph node (SLN)(FIG. 1a).

A portion of the EVs that enter the SLN continue on to be captured in the venous drainage and ultimately enter the general blood circulation. Despite the critically important biologic role of EVs shed into tissue IF, the molecular composition, function, and traffic of EVs from tissue into the lymphatic drainage through the IF are very poorly understood (Stacker et al., 2014).

In an embodiment, we set out to develop a model system for use by the general EV community to study tissue IF EVs in a murine immunocompetent model. We employed a transplantable tumour model because we could verify that the IF EVs were derived from the tumour cells in vivo by using GFP-labelled 4T1 murine syngeneic breast cancer cells. This metastatic tumour is a murine model of human triple-negative breast cancer. An advantage of this model is that we can readily culture the 4T1 tumour cells in vitro in order to compare in vivo IF versus in vitro molecular differences in EV composition.

In an embodiment, we can use insights from the EV molecular characterization of tumour tissue IF to pose experimental questions that employ the matched tumour cells in vitro first and can be subsequently reviewed by the in vivo model. Instead of attempting to dissociate and culture ex vivo tumour tissue to collect IF EVs, which could drastically alter the EV composition from that existing in situ, we gently harvested the resident IF EVs present in the tumour tissue immediately at the time of ex vivo tissue procurement by low speed centrifugation (FIG. 1b). This did not perturb the tumour cytomorphology and did not induce tumour cell death (FIG. S1). We then applied differential ultracentrifugation, density gradient separation, immunoprecipitation, and a variety of established methods to isolate and characterize the specific EVs (Demarino et al., 2018; Pinto et al., 2019, 2021).

To our knowledge, this is the first time a breast cancer model is successfully used to isolate IF EVs from an immunocompetent animal model. We have done pilot studies to estimate reproducibility of GFP expression between tumours isolated both inter- and intra-mouse (FIG. S8), and found that GFP expression is highly reproducible, indicating the EV extraction protocol is well-suited for further studies. Overall, this method is suitable for precise molecular characterization of the highly concentrated EVs shed at the tumour origin in the IF, compared to the diluted circulating tumour-derived EVs.

In an embodiment, after qualifying and characterizing the IF EVs in vivo, we compared the protein contents of the IF EVs to cell culture-derived EVs by mass spectrometry, and functionally characterized subpopulations by immunoblotting, TEM, and endothelial tube formation analyses. Our data confirms that IF EV populations and culture-derived EV populations have many differences, as expected. Nevertheless, they share a number of conserved protein biomarkers and multiple biologically relevant pathways that are represented consistently between both sources of EVs.

Proteomic characterization of IF fluid harvesting from different tumours, different healthy or diseased tissues, and the comparison with matched cell culture lines has to deal with vastly different total protein content. Moreover, while the proteins in IF are derived from tissue cell export and plasma filtrate, the total protein content of cell culture-derived material is dominated by FBS proteins added to the culture media. In the present study, we used EV-depleted media and adjusted culture cell densities and culture volumes for EV characterization to yield a similar range of EV particles/ml as found in the IF tumor tissue fluid.

In an embodiment, we chose to normalize the samples for proteomic analysis against total volume (10 μl) for multiple reasons. First, normalizing against total protein is inconsistent between EV subpopulations since EV subpopulations can contain different protein content per EV particle when measured by standard Bradford chemistry. Secondly, the cell culture contains FBS, which significantly alters measured protein concentration compared to tumour IF. Thirdly, the total number of tumour or host cells for a given solid tumour in contact with the IF is unknown, therefore making normalization by tumour tissue cell count difficult.

In an embodiment, the proteomic diversity of the culture compared to IF samples are vastly different due to the metabolic stress, cell cycle difference, and hypoxia placed upon the in vivo tumour microenvironment cells. For these reasons, important comparisons presented in FIGS. 3, 4 between the in vivo IF and the culture media are normalized by volume with the goal to elucidate specific types and classes of proteins that are differentially found in the two fluid sources. For general applications of the method, it is valuable to confirm the diversity of protein identities by MS using a normalization method.

In an embodiment, to further standardize our proteomic data, we normalized our tumour IF EVs to culture-derived EVs against the total protein peptide abundance, as shown in Figure S3(a-b). We also used this same method to normalize IF samples between different tumours (Figure S3c-d). Normalization demonstrates that a comparable dynamic range of protein identities, before versus after normalization, can be achieved by the well accepted MS normalization to total peptide abundance.

In an embodiment, as reported in FIG. S4, the 4T1 model can serve as a platform to evaluate hypotheses related to lymphatic delivery of EVs to the SLN. As shown in FIG. 2c we reported that IF 100K EVs are enriched for PD-L1. It has been postulated that EVs positive for PD-L1 in this same 4T1 model travel to the SLN to suppress host immune cell recognition of the upstream tumour (Poggio et al., 2019). Therefore, as a demonstration of the utility for the model, we sensitized the murine popliteal SLN by upstream injection of NPs containing immune cell chemoattractant CKs and 4T1 EV populations that accumulate in the SLN subcapsular sinus to release their cargo (Popova et al., 2015; Teunis et al., 2017).

The chemoattractant draws massive numbers of immune cells including dendritic cells into the SLN to potentially overcome the PD-L1 EV suppressor function. in vivo syngeneic orthotopic tumour transplantation studies show that SLN preexposure of larger 2K EVs stimulates primary tumour growth and metastasis compared to suppression of tumour grow and distant metastasis by SLN pretreatment with a matching number of 100K EVs (FIG. S4). A potential reason that the 2K fraction stimulates tumour growth is the content of VEGF (FIGS. S5 and S6) that induces vascular sprouts and angiogenesis. Further characterization of the molecular determinants for the functional differences between the pretreatment with the 2K subpopulation versus the 100K subpopulation is ongoing.

It is unclear if the immune cell priming was elicited by the presence of PD-L1 or by other 100K EV factors. Nevertheless, this model offers a potential strategy to study the role of EV subpopulations on the PD-L1+EV cancer suppression of the SLN immune recognition of the tumour. Further investigation of this model to explore EV based cancer immunotherapy is warranted for future studies.

In an embodiment, our results indicated that IF EVs are numerous and have a rich content of autophagy and mitophagy specific proteins. In recent years it has been documented that autophagy can function through an unconventional secretory pathway where instead of degradation at the lysosome, the autophagosomes are released through the cell membrane to enter the extracellular environment where they contribute to the repertoire of EVs.

Secreted autophagosomes have be found to contain inflammatory markers such as IL-1B and IL-6 as well as whole viruses and bacteria (Dupont et al., 2011; New & Thomas, 2019). Cancer cells can increase the autophagic pathway flux as a means of survival in the face of stress (Espina et al., 2010). When the autophagic flux is too high to accommodate the limits of cellular lysosomal fusion docking sites, unconventional secretion of the autophagosomes could be a compensatory mechanism. Unconventional protein secretion (UPS) mechanisms are known to be upregulated in cancer cells to export various factors that support cancer growth, progression, and eventual metastasis. Of the four known UPS systems, EVs are secreted by cells under Type III UPS mechanisms. Type III UPS mechanisms rely on leaderless molecular intermediates such as LC3-1/II and p62 to form autophagy-related secretory vesicles (New & Thomas, 2019).

Our molecular characterization of the IF EVs show the EV-associated export of autophagy and mitophagy specific molecules released through Type III UPS mechanisms (summarized in FIG. 8). Our data suggests that the EVs released by the breast cancer cells contain autophagy-related vesicular structures indicative of secretory autophagy.

Based on the literature and our evidence, we hypothesis that the 2K EVs contain secretory amphisomes, which are autophagosomes fused with late endosome/multivesicular bodies (MVB). This was supported by our TEM visualization (FIG. 5c, FIG. S7). The amphisome has partially degraded autophagosome structures within (Eskelinen, 2005; Patel et al., 2013; Sanchez-Wandelmer & Reggiori, 2013). In contrast, the 100K CD81+ EVs contain non-degraded autophagy-related vesicular structures with trafficking molecules such as SEC22B and Rab33B that are associated with protein secretion rather than degradation (Table 3) (Morgan et al., 2019; New & Thomas, 2019). We hypothesize that the 100K EVs may be trafficked to the plasma membrane for release or packaged into a MVB destined for secretion.

Within the 100K EV subpopulation, we found evidence of fission mitophagy, specifically the presence of PINK1, TOM20, and E3 ubiquitin ligases which work together to drive mitophagy (Table 4, FIG. 8). PINK1 is the major initiator of mitophagy and typically has been studied to initiate the E3 ubiquitin ligase Parkin to trigger the autophagy machinery to clear damaged mitochondria. Since Parkin is mainly present in neuronal cells, researchers have begun to uncover structurally similar E3 ubiquitin ligases involved in mitophagy for other cell types.

Our data supports the cellular function of Parkin-independent mitophagy pathways, which use other E3 ubiquitin ligases, such as ARIH1 (Di Rita et al., 2018; Villa et al., 2017, 2018). Furthermore, Parkin-dependent mitophagy has been implicated as a noted tumour suppressor (Cesari et al., 2003; Chourasia et al., 2015; Liu et al., 2017); therefore, our findings of several Parkin-independent mitophagy intermediates secreted within tumour IF EVs represent new mechanisms of mitochondrial regulation that can potentially promote tumour growth and survival.

Cancer cells are known to use autophagy as a means to survive under hypoxia, metabolic deprivation, and treatment assaults (Espina et al., 2010). Additionally, they adapt and rewire themselves metabolically to meet the high energy and high stress demands they need to survive (Macleod, 2020). During this transition, cancer cells may be required to cull defective or damaged mitochondria to meet these demands. In a hypoxic environment, the cell may be required to reduce the number of mitochondria within a cell to maintain oxygen levels and reduce reactive oxygen species (ROS) production. Further, the accumulation of damaged mitochondria can promote the creation of the inflammasome by the release of mitochondrial DNA which is highly deleterious to the cell (MacLeod, 2020). Under this elevated autophagic environment, the cancer cell may preferentially utilize the Type III UPS system to remove intracellular material, since endosomes, autophagosomes, and lysosomes have autophagy-related vesicular structures conserved between them (New & Thomas, 2019).

In an embodiment, consequently, under these high stress conditions, we posit that EVs are released via secretory autophagy rather than conventional MVB endosomal release mechanisms.

In an embodiment, we hypothesize that cancer cells utilize EVs released from the secretory autophagy and mitophagy pathways as a means to survive intracellular stresses while also aiding the tumour by the release pro-tumour markers extracellularly. This hypothesis is supported by our results that IF EVs contain the damage associated molecular pattern (DAMP) high motility box group 1 (HGMB1) and the immune suppressor molecule PD-L1 (Table 3—Inflammation, Angiogenesis, & Immune Activation) (Chen et al., 2018b; Zhao et al., 2018b; Othman et al., 2019; Poggio et al., 2019; Song et al., 2019; Tang et al., 2010).

The concept of cell metabolic or hypoxic stress as an inducer of Parkin-independent “secretory fission mitophagy” is supported by FIGS. 6e-g in which induction of mitophagy by CCCP treatment or cell crowding stress was associated with an increased export of PINK1 and MitoTracker preferentially into the CD81+ 100K EV population. The augmented content of PINK1 and MitoTracker in the CD81+ EVs was not simply due to an increased total protein or total GFP labelling of EVs (FIG. 7b) when the cells were under stress.

Under unstressed basal cellular conditions, PINK1 is imported into polarized mitochondria through the mitochondrial translocases of the outer and inner membranes (TOM and TIM), via the PINK1 amino-terminal mitochondrial targeting sequence (Onishi et al., 2021; Rasool et al., 2022). Following the import of PINK1, it is cleaved several times when it is in the inner mitochondrial membrane: first by the matrix processing peptidase (MPP) that removes the mitochondrial targeting sequence, with further cleavage by the inner membrane protease Presenilin-Associated Rhomboid-Like protein, generating a series of characteristic fragments (Becker et al., 2012; Choubey et al., 2022; Deas et al., 2011).

Further ubiquitination and degradation of cleaved PINK1 can follow to create additional PINK1 fragments (Choubey et al., 2022). In the face of mitochondrial stress, or metabolic challenge, the corrective response can be biogenesis of more mitochondria, or excision and mitophagy of functionally impaired regions of mitochondrial. Fission of mitochondria at midzone sites is triggered by mitochondrial fission factor (MFF) and precedes mitochondrial biogenesis (Kleele et al., 2021). In contrast, fission of mitochondria at peripheral sites precedes mitochondrial mitophagy, whereby the excised region is not re-fused with healthy mitochondria, and proceeds to mitophagic breakdown. Stress induced (e.g., hypoxia, nutrient deprivation, mitochondria DNA damage) mitophagy is preferentially associated with FIS1 triggered peripheral fission, but not midzone fission (Ihenacho et al., 2021., Kleele et al., 2021).

In an embodiment, In keeping with this specific class of peripheral fission leading to mitophagy, FIS1, but not MFF, was found in the 100K IF EVs (Table 4). Under stressed and depolarized conditions, mitochondrial damage results in full length PINK1 import arrest, and arrest of full length PINK1 on the TOM complex, causing the activation of its ubiquitin kinase activity (Choubey et al., 2022).

Our results (FIG. S9b) clearly show that the CD81+ tumour IF EV population contains both full length PINK1 and TOM20 with matching relative abundance in all examined tumour IF samples.

These findings support the conclusion that these 100K IF EVs contain mitochondria fragments derived from one or more stages of mitochondrial peripheral fission and downstream mitophagy. These experiments all point to the extracellular export of mitochondrial units of proteins associated with cellular stress and mitochondrial quality control, and that the relative abundances and fragment patterns could contain information about the metabolic state of the tumour. Although the data is highly consistent concerning their fission and mitophagy-associated content, we cannot know the exact type of EVs housing these proteins, and it is likely that there may be different types of EVs reflecting different stages of the peripheral fission triggered mitophagy cascade. It has recently been reported that whole mitochondria can be shed into the blood stream (Stephens et al., 2020).

Therefore, we cannot rule out that the export of mitophagy-associated EVs may potentially contain whole segments or fragments of mitochondria, with some fragments encased in EVs. The full definitive characterization of these EVs is ongoing. The cell export of mitochondria and mitochondrial-derived subfractions into EVs should provide a rich set of new information for future studies. These data support the hypothesis that IF tumour EVs with mitophagy specific contents are enriched because the tumour tissue is hypoxic and under metabolic stress, a known state of growing solid tumours (Onishi et al., 2021). As shown in FIG. 8, the multiple mechanisms of EV release employed by the tumour cells could create distinct populations of EVs, leading to the diverse biological effects reported herein, as well as clarifying the wide range of diverse and sometimes contradictory functions assigned to tumour EVs in the literature.

This study has potential limitations. eGFP labelling of the tumour cells is not identical to the native tumour and can theoretically cause some immune recognition of the tumour beyond that for unlabelled 4T1 (Bosiljcic et al., 2011). Nevertheless, as shown in FIG. S4, the lymph node priming with CD81+/PINK1+ eGFP-4T1 EVs suppressed tumour growth of native 4T1 tumours, compared to SLN priming with 2K EVs, thus demonstrating the lack of a requirement for GFP tumour content for this differential response. Alternatives to GFP-expressing cell lines, such as luciferase labelled cell lines, could be used to explore labelling of IF EVs, that can extended in future studies to the trafficking of the IF EVs through the SLN and into the peripheral vascular circulation (Baklaushev et al., 2017).

Our proteomic data results for specific IF EV-associated markers were observations made from one type of murine syngeneic tumour. Normalization methods for the EV IF MS data, support the reliability and reproducibility of the IF harvesting, and provide confidence that the method can be applied to any tissue or tumour type (FIG. S3). Although the harvesting of IF EVs and the lack of histologic impact of the IF harvesting method on the growing solid tumour was highly reproducible in our hands, we did not study large necrotic tumours or tumours post chemotherapy. Western blot results from the MDA-MB-231 human breast cancer lines indicate similar EV and mitophagy markers present within cell-cultured EVs (FIG. 6a), specifically, CD9, CD81, p62, and LC3-I/II. Density gradient separation followed by immunoprecipitation with anti-CD81 antibodies verified the presence of PINK1 in this EV gradient fraction.

We did not study the IF EVs using the MDA-MB-231 cells in a human xenograft tumour model using immunosuppressed mice. Thus, we cannot separate out the role of the host immune system on the IF EV molecular composition for human tumour cells. Additional functional analyses involving inducing and suppressing autophagy and mitophagy, both in vitro and in vivo, are needed to better understand the cellular dynamics of secretory autophagy and mitophagy. Future studies exploring the specific trafficking molecules, such as Sec22B or Rab33a, that target a vesicle for secretion rather than degradation is needed. Further research is required regarding the impact of Parkin-independent mitophagy pathways and the impact of secreted (exported from living cells) mitophagy-associated EVs on cancer progression, and the value of correlating mitophagy specific PINK1 cargo in tumour resident IF EVs as a surrogate marker for tumour responses to therapy-inducing mitochondrial stress.

In an embodiment, EVs harvested from the IF contain previously unrecognized autophagosome and mitophagy properties as well as a highly specific set of fission-mitophagy inducers and intermediates for Parkin-independent mitophagy. These secretory autophagosomes represent a potentially new biologic population of EVs that are external indicators of the metabolic state of the cell and may have a variety of important immunologic and non-immunologic functions in cancer that are associated with defective mitophagy.

Materials and Methods

Cells

The 4T1-eGFP-Puro (Imanis Life Sciences) mammary carcinoma cell line was cultured in RPMI-1640 media (Quality Biological) supplemented with 10% heat-inactivated exosome-free foetal bovine serum (FBS) (Peak Serum), 2 mM L-glutamine (Quality Biological), 100 ug/ml streptomycin (Quality Biological), 100 U/ml penicillin (Quality Biological), and 2 ug/ml puromycin (Invivogen). The MDA-MBA-231 (ATCC) adenocarcinoma cell line was cultured in Leibovitz's L-15 (ATCC) media supplemented with 10% heat-inactivated exosome-free foetal bovine serum (FBS) (Peak Serum), 2 mM L-glutamine (Quality Biological), 100 ug/ml streptomycin (Quality Biological), and 100 ug/ml penicillin (Quality Biological) for 5 days in a flask (at 37° C. and 5% CO2) before harvesting for downstream experiments. For the SLN EV immunogen priming model, the 4T1 (ATCC) mammary carcinoma cell line was cultured in RPMI-1640 (Quality Biological). When reaching confluency, cells were removed by scraping and resuspended in media after cell viability was determined by Trypan Blue exclusion.

Growth of GFP-4T1 Tumours and EV Isolation from Resident Tumour Interstitial Fluid (IF) Ex Vivo

4T1-GFP cells at 1×106/ml were injected into the mammary fat pads of 6-week-old BALB/c mice (Charles River Laboratory). The animals were sacrificed after 4 weeks, and the tumours were excised. Following removal, the tumours were placed in an EconoSpin column lined with glass wool and suspended in 300 μl of PBS. This assembly was then centrifuged at 8160×g for 10 min. The flow through was harvested and then centrifugated at 2000×g for 45 min at 4° C. The resulting “2K” pellet was harvested and the supernatant was centrifuged at 10,000×g for 45 min at 4° C. The “10K” pellet was harvested followed by the supernatant collected and treated with nanotrap particles. Nanotrap particles (NTs) Ceres #CN1035, and Ceres #CN2010 (Ceres Nanosciences, Inc.) were used to enrich EVs from low volume, cell-free supernatant samples as previously described (Ahsan et al., 2016; Demarino et al., 2018; Narayanan et al., 2013; Pleet et al., 2016, 2018; Sampey et al., 2016). One hundred microliters of the CN1035/2010 slurry (30% CN1035, 30% CN2010 and 30% PBS) were added into the 10,000×g depleted supernatant and allowed to rotate at 4° C. overnight. The NTs were then harvested by centrifugation at 20,800×g for 10 min at 4° C. resulting in the “100K*” pellet. These samples were later utilized in Western Blot and mass spectrometry.

EV Isolation by Differential Ultracentrifugation

The 4T1, MDA-MBA-231, and 4T1-eGFP-Puro cell cultures were grown for 5 days, after which supernatants were collected from the cultures. The supernatants were centrifugated at 500×g for 10 min, with the resulting pellet being discarded and the resulting supernatants being collected. The supernatants were then centrifugated sequentially in a 70Ti rotor (Beckman) with the resulting pellets being saved and the resulting supernatant being used in the following centrifugations at 2000×g for 45 min at 4° C., then 10,000×g for 45 min at 4° C., and finally 100,000×g for 90 min at 4° C. The resulting pellets were denoted as “2K”, “10K”, and “100K”, respectively, and were later utilized in downstream assays.

Western Blot Analysis

Whole-cell extracts (10 μg) or concentrated differential ultracentrifuge samples (5-10 μl) were resuspended in 10 μl of Laemmli buffer, heated at 95° C. for 3 min, and loaded onto a −20% Tris-Glycine SDS gel (Invitrogen). CN1035/2010 NT pellets were resuspended in 15-20 μl of Laemmli buffer then heated at 95° C. for 3 min and vortexed repeating these steps three times until fully resuspended. The eluted material was then loaded onto a 4-20% Tris-Glycine gel. Gels were run at 100 V and wet-transferred overnight at 50 mA onto polyvinylidene difluoride (PVDF) membranes. Membranes were blocked in 5% milk in PBS-1× containing 0.1% Tween 20 (PBST) for a 1 h at 4° C., then incubated overnight at 4° C. with appropriate primary antibody in PBST at recommended manufacturer dilutions. Antibodies used for these experiments included: anti-eGFP (Abcam), anti-CD63 (Santa Cruz), anti-CD81 (Santa Cruz), anti-CD9 (Abcam), anti-β-Actin (Abcam), anti-VEGF (Thermo Fisher), anti-LC3B (Cell Signalling), anti-p62 (Cell Signalling), anti-PINK1 (Novus Biologics), and anti-PD-L1 (Rockland). Membranes were then incubated with the appropriate horseradish peroxidase (HRP)-conjugated secondary antibody for 2 h at 4° C. or for 1 h at room temperature and developed using Clarity or Clarity Max Western ECL Substrate (Bio-Rad). Luminescence was visualized on a ChemiDoc Touch Imaging System (Bio-Rad).

Mass Spectrometry

The EV samples (10 μl) were mixed with 20 μl of 8 M urea and reduced with 10 mM dithiothreitol at 50° C. for 5 min. The mixture was alkylated with 50 mM iodoacetamide at room temperature for 15 min and digested with trypsin at 37° C. for 4 h. The sample was desalted by ZipTip, dried in SpeedVac, then reconstituted with 10 μl of 0.1% formic acid for mass spectrometry (MS) analysis. Liquid chromatography coupled tandem mass spectrometry (LC-MS/MS) experiments were performed on an Exploris 480 (ThermoFisher Scientific, Waltham, MA, USA) equipped with a nanospray EASY-nLC 1200 HPLC system. Peptides were separated using a reversed-phase PepMap RSLC 75 μm i.d.×15 cm long with 2 μm particle size C18 LC column from ThermoFisher Scientific. The mobile phase consisted of 0.1% aqueous formic acid (mobile phase A) and 0.1% formic acid in 80% acetonitrile (mobile phase B). After sample injection, the peptides were eluted by using a linear gradient from 5% to 40% B over 90 min and ramping to 100% B for an additional 2 min. The flow rate was set at 300 nl/min. The Exploris 480 was operated in a data-dependent mode in which one full MS scan (60,000 resolving power) from 300 m/z to 1500 m/z was followed by MS/MS scans in which the most abundant molecular ions were dynamically selected and fragmented by higher-energy collisional dissociation (HCD) using a collision energy of 27%. “EASY-Internal Calibration”, “Peptide Monoisotopic Precursor Selection” and “Dynamic Exclusion” (15 s duration), were enabled, as was the charge state dependency so that only peptide precursors with charge states from +2 to +4 were selected and fragmented. Tandem mass spectra were searched against the NCBI human and mouse database using Proteome Discover v 2.3 from ThermoFisher Scientific. The SEQUEST node parameters were set to use full tryptic cleavage constraints with dynamic methionine oxidation. Mass tolerance for precursor ions was 2 ppm, and mass tolerance for fragment ions was 0.02 Da. A 1% false discovery rate (FDR) was used as a cut-off value for reporting peptide spectrum matches (PSM) from the database search. For protein abundance normalization to eGFP, the mass tolerance for precursor ions was adjusted to 5 ppm, and mass tolerance for fragment ions was 0.6 Da to accommodate the post-translational modifications of eGFP.

To identify the shared proteins returned between each sample, the protein GI numbers were inputted into the web-based Venn diagram maker InteractiVenn (Heberle et al., 2015). Fold-enrichments of over-expressed protein pathways by EV sample were determined by using PANTHER pathway database (Mi & Thomas, 2009).

EV-CK Nanoparticle Delivery Synthesis

Sterile N-Isopropylacrylamide (NIPAm) particles with a ˜7% molar content of acrylic acid (AAc) were synthesized by precipitation polymerization and coupled with Cibracron Blue F3G triazine dye by direct reaction between the carboxylic acid groups of the particles (Tamburro et al., 2011; Teunis et al., 2017). Cibracron Blue dyed nanoparticles (NPs) were aseptically incubated with 1 μg/ml concentration of carrier-free human CCL18/PARC (RD Systems) and 1 μg/ml of carrier-free mouse CXCL9/MIG (Biolegend) at 4° C. overnight (CK/NPs). After overnight incubation, the CK/NP mix was washed and resuspended with sterile PBS-1×. Next, the NPs were diluted 1:10 in PBS-1× and mixed equi-volume with each EV subpopulation and incubated overnight at 4° C. overnight (EV/NPs). The EV/NP mix was washed and resuspended with sterile PBS-1×.

Sentinel Lymph Node Priming Animal Model

Six to ten-week-old BALB/c female mice were obtained from Jackson Labs. Mice were injected with 50 μl of a 1:1 ratio of CK/NPs and EV/NP subtype via subcutaneous injection in the right foot pad. Two days later, mice were challenged with 1×106 4T1 cells/mouse at the same location and monitored for spontaneous tumour growth. A control group of mice received just the 4T1 challenge. At the 4 week mark, animals were sacrificed and tissues were harvested for immunohistochemistry. Tumour growth was measured by calipers. A follow-up experiment was performed and repeated these procedures, except the treatment and tumour cell challenge were subcutaneously injected into the mammary fat pad. All animal experiments were approved by the George Mason Institutional Animal Care and Use Committee (IACUC; 1312869-1).

Angiogenesis Assay

To assess the effects of 4T1 EVs on tubular formation, the Angio-Ready Angiogenesis Assay System (ACS-2001-2; ATCC) was utilized. The assay was performed following the recommended protocol and experimental samples were assayed in triplicate. On day 0, the assay was initiated in fully supplemented Angio-Ready Angiogenesis Medium (ACS-2008; ATCC). On day 2, the medium was removed and replaced with a 1:2 dilution of the fully supplemented medium. Treatment with EVs was based on an approximate ratio of 1:2000 (recipient cell to EV ratio) while untreated cells received only PBS. Cells were re-treated again on day 4. A fluorescent microscope was used to image the cells at various time points and image analysis was performed using the Wimasis WimTube analysis platform.

EV Uptake Assay

The 4T1 EVs were labelled with BODIPY 493/503 (Thermo Fisher) as per the manufacturer's protocol. Briefly, various EV samples (50-100 μl) were labelled for 1 h at 37° C. (˜109-1010 EVs/5 μl of dye). Samples were then passed through a Sephadex G-50 column (1 ml syringe; 0.5 ml bed volume adjusted with PBS buffer) at 2000 rpm for 2 min. The columns were washed with 25 μl of PBS at the same speed for 2 min and the collected EVs were counted for further analysis. The unincorporated dyes were trapped at the upper ⅓ of the column and EVs were collected in the flow-through. Labelled EVs were then added to confluent cultures of ASC52telo hTERT-immortalized MSCs (SCRC-4000; ATCC). EVs were added at an approximate ratio of 1:2000 (recipient cell to EV ratio) and incubated at 37° C. for a period of 6 days. Images were captured using a fluorescent microscope.

Electron Microscopy

EV subpopulations were imaged via transmission electron microscopy (TEM) using the in-block preparation method by Jung and Mun, with minor modifications (Jung & Mun, 2018). Briefly, pelleted EV subpopulations were fixed in 1 ml of 2.5% glutaraldehyde in 0.1 M sodium cacodylate buffer overnight at 4° C. Pellets were washed in 0.1 M sodium cacodylate, then post-fixed in 1 ml of 2% osmium tetroxide in 0.1 M sodium cacodylate buffer for 1 h at 4° C. Pellets were washed again in 0.1 M sodium cacodylate buffer then dehydrated with a graded acetone series. Dehydrated pellets were resin embedded in Spurr Low Viscosity resin (Sigma-Aldrich EM0300-1KT) via a graded series of 3:1, 1:1, and 1:3 acetone:resin, then dried overnight at 65° C. in 100% resin in Beem capsules. For each EV subpopulation, 60-100 nm sections were cut on a ultramicrotome and collected on Formvar/carbon film copper TEM grids, then double stained in 2% uranyl acetate followed by Reynold's lead citrate. Sections were imaged at 200 kV on a JEOL JEM 2100-Plus.

Pooled Izon column fractions 11-15 (PINK+) were imaged via scanning electron microscopy. Sample (3 uL) was spotted on a silicon chip and dried under vacuum overnight. Samples were imaged uncoated on a JEOL JSM-7200F between 3 kV and 5 kV.

EV Isolation by Iodixanol Gradient Ultracentrifugation and Immunoprecipitation

Five-day-old supernatants were collected from 4T1-eGFP cells or MDA-MB-231 cells and centrifugated at 500×g for 10 min. The resulting supernatants were then incubated with ExoMax (System Biosciences) at a 1:1 ratio overnight at 4° C. After incubation, the samples were centrifugated at 2000×g for 30 min with the resulting pellet being resuspended in 300-400 μl of PBS. Eleven iodixanol gradient fractions (1 ml/fraction; 6.0-18.0% iodixanol with a 1.2% iodixanol increase between fractions) were created by adding iodixanol to PBS in the appropriate ratio. These fractions were then loaded into a centrifuge tube compatible with the Beckman SW41 rotor in order of highest density (18.0%) to lowest density (6.0%). This was followed by the resuspended pellet collected from the ExoMax preparation. These samples were then centrifugated at 100,000×g for 90 min at 4° C. The volume of the resuspended pellet and each 1 ml fraction were removed and then stored in microcentrifuge tubes. These fractions were used in various downstream assays.

Select iodixanol fractions, 250 μl, were rotated with 2 μg of antibody, either anti-CD81 or anti-ERCC1 (D-10) (Santa Cruz) overnight at 4° C. Samples were then incubated with 20 μl of Protein G Plus/Protein A Agarose Suspension (Millipore Sigma) for 2 h at 4° C. These samples were centrifuged at 15,000×g for 10 min at 4° C. The resulting pellet was then used in Western blots.

Mitochondrial Oxidative Stress Perturbation

Two 4T1-eGFP cell cultures were plated at 106 cells/ml. Untreated cells were left to grow for 5 days. Treated cells were stimulated with 10 nM of carbonyl cyanide 3-chlorophenylhydrazone (CCCP) (Sigma) and left to grow for 5 days. The supernatants were collected and EVs fractions were isolated by differential ultracentrifugation. The resulting 100K EVs were probed for anti-ARIH1 (Santa Cruz) by Western blot.

Mitochondrial EV Fluorescent Labelling

Two 4T1-eGFP-Puro cell cultures were plated at 106 cells/ml. Untreated cells were left to grow for 48 h. Treated cells were stimulated with 10 nM and 10 μM of CCCP and left to grow for 48 h. At 48 h, 50 nM of MitoTracker Deep Red (MTDR) (Thermo Fisher) was added to the media and left to sit overnight in the cell incubator. The supernatants were collected and EVs were isolated by differential ultracentrifugation. EVs subpopulations were plated at 30 μl and scanned for peak emission fluorescent intensity fluorescence at 450/490 nm (absorbance/emission) for GFP and 635/675 nm (absorbance/emission) for MTDR on a Tecan safire2 plate reader.

EV Protease Analysis

Tumour IF that has been depleted for 2K and 10K subpopulations by centrifugation and validated for PINK1 and CD81 positivity (10 μg) were incubated with dilutions of trypsin (Promega) for 30 min at 37° C. After incubation, 15 μl of Laemmli buffer was added to trypsin-treated and control samples. The samples were analysed via Western Blot.

EV Free Protein Separation Analysis

To understand the free protein content in the EV isolation we used our previously published protocol (Pinto et al., 2021). Five-day-old supernatants were collected from eGFP-4T1 cells and centrifugated at 500×g for 10 min, with the resulting pellet being discarded. The resulting supernatant was sequentially centrifugated to remove the 2K and 10K EV subpopulations. The resulting supernatant was then mixed with ExoMax™ (System Biosciences) at a 1:1 ratio overnight at 4° C. to enrich and precipitate the EVs to reduce the free protein. After incubation, the samples were centrifugated at 2000×g for 30 min with the resulting pellet being resuspended in 300-400 μl of PBS. Next, 200 μl of concentrated material was loaded onto a qEV/35 nm size exclusion column (IZON). The resulting flow through material was collected into forty 200 μl fractions. 100 μl of each fraction was pooled into groups of five for a total of 500 μl. Each fraction's protein content was analysed via Bradford assay. Next, the pooled fractions were incubated overnight at 4° C. with CN1035/2010 (Ceres Nanosciences). NTs were pelleted and prepared for Western blot analysis. Gel electrophoresis protein detection was performed by Silver Stain according to manufacturer instructions (Pierce Silver Stain Kit, Thermo Fisher). Densitometry analysis of the protein amount was quantified by ImageJ.

Statistical Analysis

Standard deviation was calculated in all quantitative experiments done in triplicate. All p-values were calculated using two-tailed Student's t-tests or one-way ANOVA as indicated. Differences were considered statistically significant when p<0.05. All statistical analyses were performed using GraphPad Prism Software version 8.3.1.

### INCORPORATION BY REFERENCE

All publications, patents and patent applications cited in this specification are incorporated herein by reference in their entireties as if each individual publication, patent or patent application were specifically and individually indicated to be incorporated by reference. While the foregoing has been described in terms of various embodiments, the skilled artisan will appreciate that various modifications, substitutions, omissions, and changes may be made without departing from the spirit thereof.

TABLE 3
Key functional roles of proteins returned by subtype from the T1 tumour

| | | | Presence | | | |
|---|---|---|---|---|---|---|
| | Protein | Function | 2K | 10K | 100K | Reference |
| Inflammation, | Fibroblast growth factor | Common Angiogenic activator | 0 | 0 | 1 | Pircher et al., 2011 |
| angiogenesis, | receptor 3 | | | | | |
| and immune | Hepatoma-derived | Pro-angiogenic factor | 0 | 1 | 1 | Leblanc et al., 2015 |
| activation | growth factor-related | | | | | |
| | protein 3 | | | | | |
| | Annexin II | Exosomal Annexin II has been associated with | 1 | 1 | 1 | Maji et al., 2017 |
| | | increased angiogenesis and ECM remodelling, | | | | |
| | | and increased metastasis | | | | |
| | Tumour necrosis factor | Exosomal Pro-inflammatory cytokine which | 0 | 0 | 1 | Othman et al., 2019 |
| | alpha-induced protein | increases immune cell activation to aid in cancer | | | | |
| | | growth, invasion, and metastasis | | | | |
| | CD44 | Important cancer cell signalling protein | 0 | 1 | 1 | Chen et al., 2018a |
| | | involved in stimulated angiogenesis, metabolic | | | | |
| | | shift, and cancer cell proliferation. | | | | |
| | High mobility group | Damage associated molecular pattern molecule | 0 | 1 | 1 | Tang et al., 2010 |
| | protein B2 | which has been noted to induce autophagy and | | | | |
| | | affect mitophagy | | | | |
| | Programmed death | Cancer cell surface expression check-point leads | 0 | 0 | 1 | Poggio et al., 2019 |
| | ligand 1 (PD-L1) | to immunosuppression of emerging cancer | | | | |
| | Mitogen-activated | Pro-cancer kinase which induces cell | 0 | 0 | 1 | Mashouri et al., 2019 |
| | protein kinase 1 (MAPK) | proliferation and growth | | | | |
| | E3 ubiquitin-protein | Ubiquitin ligase which assists LC3 in | 1 | 1 | 1 | Sun et al., 2017 |
| | ligase NEDD4 | autophagosome biogenesis | | | | |
| | Sequestosome-1 | Key autophagy substrate that targets molecules | 0 | 1 | 0 | Katsuragi et al., 2015 |
| | | for autophagy. | | | | |
| | Ras-related protein Rab- | Involved in autophagosome biogenesis | 0 | 0 | 1 | Morgan et al., 2019 |
| | 3D | | | | | |
| | Ras-related protein Rab- | Key autophagic trafficking GTPase. noted to | 0 | 1 | 1 | Morgan et al., 2019 |
| | 33B | regulate/control exophagic viral release in HBV | | | | |
| Autophagy | Ras-related protein Rab- | Overexpression of Rab-4a leads to formation of | 0 | 0 | 1 | Morgan et al., 2019 |
| | 4A | autophagosome | | | | |
| | Ras-related protein Rab- | Aids in ULK1 translocation | 1 | 1 | 1 | Morgan et al., 2019 |
| | 1A | | | | | |
| | Ras-related protein Rab- | Promotes ATG5-ATG12 conjugation and | 0 | 0 | 1 | Morgan et al., 2019 |
| | 5C | therefore autophagosomal elongation | | | | |
| | Ras-related protein Rab- | Aids in autophagosome clearance | 0 | 0 | 1 | Morgan et al., 2019 |
| | 2A | | | | | |
| | Vesicle-trafficking | Trafficking protein involved in IL-1B secretory | 0 | 1 | 1 | Dupont et al., 2011 |
| | protein SEC22B | autophagy | | | | |

TABLE 4
IF EVs contain markers for Parkin-independent mitophagy.

| | | | Sub- | | | Presence | | | |
|---|---|---|---|---|---|---|---|---|---|
| | | Pathway | pathway | Protein | Function | 2K | 10K | 100K | Reference |
| | | | | Voltage- | Outer membrane | 0 | 1 | 0 | Camara et al., 2017; |
| | | | | dependent | mitochondrial metabolic | | | | De Paepe, 2012 |
| | | | | anion-selective | porin that functions as a | | | | |
| | | | | channel protein | receptor for pro- and | | | | |
| | | | | 1 | anti-apoptotic proteins. | | | | |
| | | | | Mitochondrial | Translocase which | 0 | 0 | 1 | Rasool et al., 2022 |
| | | | | import receptor | imports porteins into the | | | | |
| | | | | subunit | intermembrane space as | | | | |
| | | | | TOM20 | well as dimerizes with | | | | |
| | | | | | PINK1 under | | | | |
| | | | | | mitochondria stress to | | | | |
| | | | | | facilitate mitophagy. | | | | |
| Mitochondria | Membrane | | | Mitochondrial | Co-chaperone of HSP70 | 0 | 0 | 1 | Faou & |
| | Proteins | | | import receptor | and HSP90 involved in | | | | Hoogenraad, 2012 |
| | | | | subunit | mitochondrial protein | | | | |
| | | | | TOM34 | import. | | | | |
| | | | | Hsp90 co- | Chaperones invovled in | 1 | 1 | 1 | Tang et al., 2010 |
| | | | | chaperone | ULK1 complex | | | | |
| | | | | Cdc37 | stabilization and | | | | |
| | | | | | activation | | | | |
| | | | | Vacuolar | Controller of retrograde | 0 | 1 | 1 | Braschi et al., 2010; |
| | | | | protein sorting- | trafficking of cargo | | | | Cutillo et al., 2020; |
| | | | | associated | proteins from the | | | | Roberts et al., 2016 |
| | | | | protein 35 | endosome to the trans- | | | | |
| | | | | | Golgi network. | | | | |
| | | | | | Specifically, invovled in | | | | |
| | | | | | mitochondria-derived | | | | |
| | | | | | vesicle formation and | | | | |
| | | | | | mitochondrial fission. | | | | |
| | | Parkin- | | PTEN-induced | Mitochondrial | 1 | 1 | 1 | Macleod, 2020 |
| | | dependent | | putative kinase | depolarization causes | | | | |
| | | | | 1 (PINK1) | PINK1 to accumulate on | | | | |
| | | | | | the outer membrane to | | | | |
| | | | | | phosphorylate ubiquitin | | | | |
| | | | | | to Ubiquitin ligases to | | | | |
| | | | | | the mitochondrial outer | | | | |
| | | | | | membrane | | | | |
| | | | Receptor- | N.F. | | | | | Villa et al., 2018 |
| | | | mediated | | | | | | |
| | | | mitophagy | | | | | | |
| | | | Lipid- | N.F. | | | | | Villa et al., 2018 |
| | | | mediated | | | | | | |
| | | | mitophagy | | | | | | |
| | | | | E3 ubiquitin- | Regulator of mitophagy | 0 | 0 | 1 | Melino et al., 2019 |
| | | | | protein ligase | which localizes on the | | | | |
| | | | | SMURF1 | outer membrane to | | | | |
| | | | | | trigger autophagy | | | | |
| | | | | | machinery to damaged | | | | |
| | | | | | mitochondria | | | | |
| | | Parkin- | E3 | E3 ubiquitin- | Inducer of AMBRA1 | 0 | 0 | 1 | Di Rita et al., 2018; |
| | | independent | ubiquitin | protein ligase | mediated mitophagy | | | | Melino et al., 2019 |
| | | | ligases | HUWE1 | | | | | |
| Mitochondria | Mitophagy | | | E3 ubiquitin- | Parkin-independent | 0 | 0 | 1 | Villa et al., 2017, |
| | | | | protein ligase | mitophagy regulator | | | | 2018 |
| | | | | ARIH1 | activated by PINK-1 | | | | |
| | | | | | phosphorylation to | | | | |
| | | | | | initiate p62 induced | | | | |
| | | | | | mitophagy | | | | |
| | | | | mitochondrial | Mitochondrial protein | 1 | 0 | 1 | De Paepe, 2012 |
| | | | | fission 1 | involved in | | | | |
| | | | | protein (FIS1) | fragmentation process | | | | |
| | | | | | associated with organelle | | | | |
| | | | | | fission or mitophagy | | | | |
| | | | Peripheral | dynamin-1-like | GTPase enzyme that | 1 | 0 | 1 | Roberts et al., 2016; |
| | | | Fission | protein (DLP1) | defines mitochondrial | | | | Yoon et al., 2003 |
| | | | | | fission location with | | | | |
| | | | | | FIS1 to form a | | | | |
| | | | | | constricting ring-like | | | | |
| | | | | | structure. | | | | |
| | | | | mitochondrial | Protein which initiates | 0 | 0 | 0 | Kleele et al., 2021 |
| | | | | fission factor | mid-zone mitochondrial | | | | |
| | | | | (MFF) | fission. | | | | |
| | | | Midzone | dynamin-1-like | GTPase enzyme that | 1 | 0 | 1 | Roberts et al., 2016; |
| | | | Fission | protein (DLP1) | defines mitochondrial | | | | Yoon et al., 2003 |
| | | | | | fission location with | | | | |
| | | | | | FIS1 to form a | | | | |
| | | | | | constricting ring-like | | | | |
| | | | | | structure. | | | | |

Abbreviation: N.F., not found.

### REFERENCES

Kibbe, A. H., Handbook of Pharmaceutical Excipients rd (2000)
Ahsan, N. A., Sampey, G. C., Lepene, B., Akpamagbo, Y., Barclay, R. A., Iordanskiy, S., Hakami, R. M., and Kashanchi, F. (2016). Presence of Viral RNA and Proteins in Exosomes from Cellular Clones Resistant to Rift Valley Fever Virus Infection. Front Microbiol 7.
Anstine, L. J., and Keri, R. (2019). A new view of the mammary epithelial hierarchy and its implications for breast cancer initiation and metastasis. Journal of Cancer Metastasis and Treatment 5, 50.
Baklaushev, V. P., Kilpelainen, A., Petkov, S., Abakumov, M. A., Grinenko, N. F., Yusubalieva, G. M., Latanova, A. A., Gubskiy, I. L., Zabozlaev, F. G., Starodubova, E. S., et al. (2017). Luciferase Expression Allows Bioluminescence Imaging But Imposes Limitations on the Orthotopic Mouse (4T1) Model of Breast Cancer. Scientific Reports 7, 7715.
Becker, D., Richter, J., Tocilescu, M. A., Przedborski, S., and Voos, W. (2012). Pink1 kinase and its membrane potential (Deltay)-dependent cleavage product both localize to outer mitochondrial membrane by unique targeting mode. J Biol Chem 287, 22969-22987.
Bosiljcic, M., Hamilton, M. J., Banath, J. P., LePard, N. E., McDougal, D. C., Jia, J. X., Krystal, G., and Bennewith, K. L. (2011). Myeloid Suppressor Cells Regulate the Lung Environment—Letter: FIG. 1. Cancer Res 71, 5050-5051.
Camara, A. K. S., Zhou, Y., Wen, P.-C., Tajkhorshid, E., and Kwok, W.-M. (2017). Mitochondrial VDAC1: A Key Gatekeeper as Potential Therapeutic Target. Front. Physiol. 8.
Cesari, R., Martin, E. S., Calin, G. A., Pentimalli, F., Bichi, R., McAdams, H., Trapasso, F., Drusco, A., Shimizu, M., Masciullo, V., et al. (2003). Parkin, a gene implicated in autosomal recessive juvenile parkinsonism, is a candidate tumor suppressor gene on chromosome 6q25-q27. Proc Natl Acad Sci USA 100, 5956-5961.
Chazotte, B. (2011). Labeling Mitochondria with MitoTracker Dyes. Cold Spring Harb Protoc 2011, pdb.prot5648.
Chen, C., Zhao, S., Karnad, A., and Freeman, J. W. (2018a). The biology and role of CD44 in cancer progression: therapeutic implications. J Hematol Oncol 11.
Chen, G., Wang, B., Sun, H., Xia, H., Man, Q., Zhong, W., Antelo, L. F., Wu, B., Xiong, X., Liu, X., et al. (2018b). Exosomal PD-L1 contributes to immunosuppression and is associated with anti-PD-1 response. Nature; London 560, 382-6,386A-386P.
Chiba, M., Kubota, S., Sato, K., and Monzen, S. (2018). Exosomes released from pancreatic cancer cells enhance angiogenic activities via dynamin-dependent endocytosis in endothelial cells in vitro. Sci Rep 8, 1-9.
Choubey, V., Zeb, A., and Kaasik, A. (2022). Molecular Mechanisms and Regulation of Mammalian Mitophagy. Cells 11, 38.
Chourasia, A. H., Tracy, K., Frankenberger, C., Boland, M. L., Sharifi, M. N., Drake, L. E., Sachleben, J. R., Asara, J. M., Locasale, J. W., Karczmar, G. S., et al. (2015). Mitophagy defects arising from BNip3 loss promote mammary tumor progression to metastasis. EMBO Rep 16, 1145-1163.
De Paepe, B. (2012). Mitochondrial Markers for Cancer: Relevance to Diagnosis, Therapy, and Prognosis and General Understanding of Malignant Disease Mechanisms (Hindawi).
Deas, E., Plun-Favreau, H., Gandhi, S., Desmond, H., Kjaer, S., Loh, S. H. Y., Renton, A. E. M., Harvey, R. J., Whitworth, A. J., Martins, L. M., et al. (2011). PINK1 cleavage at position A103 by the mitochondrial protease PARL. Human Molecular Genetics 20, 867-879.
DeMarino, C., Pleet, M. L., Cowen, M., Barclay, R. A., Akpamagbo, Y., Erickson, J., Ndembi, N., Charurat, M., Jumare, J., Bwala, S., et al. (2018). Antiretroviral Drugs Alter the Content of Extracellular Vesicles from HIV-1-Infected Cells. Scientific Reports 8, 7653.
DeMarino, C., Barclay, R. A., Pleet, M. L., Pinto, D. O., Branscome, H., Paul, S., Lepene, B., El-Hage, N., and Kashanchi, F. (2019). Purification of High Yield Extracellular Vesicle Preparations Away from Virus. J Vis Exp.
Di Rita, A., Peschiaroli, A., D'Acunzo, P., Strobbe, D., Hu, Z., Gruber, J., Nygaard, M., Lambrughi, M., Melino, G., Papaleo, E., et al. (2018). HUWE1 E3 ligase promotes PINK1/PARKIN-independent mitophagy by regulating AMBRA1 activation via IKKα. Nature Communications 9, 3755.
Dupont, N., Jiang, S., Pilli, M., Ornatowski, W., Bhattacharya, D., and Deretic, V. (2011). Autophagy-based unconventional secretory pathway for extracellular delivery of IL-10. EMBO J 30, 4701-4711.
Eskelinen, E.-L. (2005). Maturation of Autophagic Vacuoles in Mammalian Cells. Autophagy 1, 1-10.
Eskelinen, E.-L. (2008). To be or not to be? Examples of incorrect identification of autophagic compartments in conventional transmission electron microscopy of mammalian cells. Autophagy 4, 257-260.
Espina, V., Edmiston, K. H., Heiby, M., Pierobon, M., Sciro, M., Merritt, B., Banks, S., Deng, J., VanMeter, A. J., Geho, D. H., et al. (2008). A Portrait of Tissue Phosphoprotein Stability in the Clinical Tissue Procurement Process. Molecular & Cellular Proteomics 7, 1998-2018.
Espina, V., Mariani, B. D., Gallagher, R. I., Tran, K., Banks, S., Wiedemann, J., Huryk, H., Mueller, C., Adamo, L., Deng, J., et al. (2010). Malignant Precursor Cells Pre-Exist in Human Breast DCIS and Require Autophagy for Survival. PLoS One 5.
Faou, P., and Hoogenraad, N. J. (2012). Tom34: A cytosolic cochaperone of the Hsp90/Hsp70 protein complex involved in mitochondrial protein import. Biochimica et Biophysica Acta (BBA)—Molecular Cell Research 1823, 348-357.
Fleming, A., Sampey, G., Chung, M.-C., Bailey, C., van Hoek, M. L., Kashanchi, F., and Hakami, R. M. (2014). The carrying pigeons of the cell: exosomes and their role in infectious diseases caused by human pathogens. Pathog Dis 71, 109-120.
Gallart-Palau, X., Serra, A., and Sze, S. K. (2016). Enrichment of extracellular vesicles from tissues of the central nervous system by PROSPR. Mol Neurodegener 11.
Groza, M., Zimta, A.-A., Irimie, A., Achimas-Cadariu, P., Cenariu, D., Stanta, G., and Berindan-Neagoe, I. Recent advancements in the study of breast cancer exosomes as mediators of intratumoral communication. Journal of Cellular Physiology 0.
Heberle, H., Meirelles, G. V., da Silva, F. R., Telles, G. P., and Minghim, R. (2015). InteractiVenn: a web-based tool for the analysis of sets through Venn diagrams. BMC Bioinformatics 16, 169.
Ihenacho, U. K., Meacham, K. A., Harwig, M. C., Widlansky, M. E., and Hill, R. B. (2021). Mitochondrial Fission Protein 1: Emerging Roles in Organellar Form and Function in Health and Disease. Frontiers in Endocrinology 12.
Jang, S. C., Crescitelli, R., Cvjetkovic, A., Belgrano, V., Olofsson Bagge, R., Sundfeldt, K., Ochiya, T., Kalluri, R., and Lötvall, J. (2019). Mitochondrial protein enriched extracellular vesicles discovered in human melanoma tissues can be detected in patient plasma. J Extracell Vesicles 8.
Jung, M. K., and Mun, J. Y. (2018). Sample Preparation and Imaging of Exosomes by Transmission Electron Microscopy. JoVE (Journal of Visualized Experiments) e56482.
Katsuragi, Y., Ichimura, Y., and Komatsu, M. (2015). p62/SQSTM1 functions as a signaling hub and an autophagy adaptor. The FEBS Journal 282, 4672-4678.
Kleele, T., Rey, T., Winter, J., Zaganelli, S., Mahecic, D., Perreten Lambert, H., Ruberto, F. P., Nemir, M., Wai, T., Pedrazzini, T., et al. (2021). Distinct fission signatures predict mitochondrial degradation or biogenesis. Nature 593, 435-439.
LeBlanc, M. E., Wang, W., Caberoy, N. B., Chen, X., Guo, F., Alvarado, G., Shen, C., Wang, F., Wang, H., Chen, R., et al. (2015). Hepatoma-Derived Growth Factor-Related Protein-3 Is a Novel Angiogenic Factor. PLOS ONE 10, e0127904.
Liu, W., and Hajjar, K. A. (2016). The annexin A2 system and angiogenesis. Biological Chemistry 397, 1005-1016.
Liu, J., Zhang, C., Zhao, Y., Yue, X., Wu, H., Huang, S., Chen, J., Tomsky, K., Xie, H., Khella, C. A., et al. (2017). Parkin targets HIF-1α for ubiquitination and degradation to inhibit breast tumor progression. Nature Communications 8, 1823.
Liu, T., Han, C., Wang, S., Fang, P., Ma, Z., Xu, L., and Yin, R. (2019). Cancer-associated fibroblasts: an emerging target of anti-cancer immunotherapy. Journal of Hematology & Oncology 12, 86.
Longo, C., Patanarut, A., George, T., Bishop, B., Zhou, W., Fredolini, C., Ross, M. M., Espina, V., Pellacani, G., Petricoin, E. F., et al. (2009). Core-shell hydrogel particles harvest, concentrate and preserve labile low abundance biomarkers. PLoS ONE 4, e4763.
Lowry, M. C., Gallagher, W. M., and O'Driscoll, L. (2015). The Role of Exosomes in Breast Cancer. Clinical Chemistry 61, 1457-1465.
Luchini, A., Geho, D. H., Bishop, B., Tran, D., Xia, C., Dufour, R. L., Jones, C. D., Espina, V., Patanarut, A., Zhou, W., et al. (2008). Smart hydrogel particles: biomarker harvesting: one-step affinity purification, size exclusion, and protection against degradation. Nano Lett. 8, 350-361.
Macleod, K. F. (2020). Mitophagy and Mitochondrial Dysfunction in Cancer. Annual Review of Cancer Biology 4, 41-60.
Madu, C. O., Wang, S., Madu, C. O., and Lu, Y. (2020). Angiogenesis in Breast Cancer Progression, Diagnosis, and Treatment. J Cancer 11, 4474-4494.
Magni, R., Almofee, R., Yusuf, S., Mueller, C., Vuong, N., Almosuli, M., Hoang, M. T., Meade, K., Sethi, I., Mohammed, N., et al. (2020). Evaluation of pathogen specific urinary peptides in tick-borne illnesses. Sci Rep 10, 19340.
Maji, S., Chaudhary, P., Akopova, I., Nguyen, P. M., Hare, R. J., Gryczynski, I., and Vishwanatha, J. K. (2017). Exosomal Annexin II Promotes Angiogenesis and Breast Cancer Metastasis. Mol Cancer Res 15, 93-105.
Margolis, L., and Sadovsky, Y. (2019). The biology of extracellular vesicles: The known unknowns. PLOS Biology 17, e3000363.
Mashouri, L., Yousefi, H., Aref, A. R., Ahadi, A. mohammad, Molaei, F., and Alahari, S. K. (2019). Exosomes: composition, biogenesis, and mechanisms in cancer metastasis and drug resistance. Molecular Cancer 18, 75.
Melino, G., Cecconi, F., Pelicci, P. G., Mak, T. W., and Bernassola, F. (2019). Emerging roles of HECT-type E3 ubiquitin ligases in autophagy regulation. Molecular Oncology 13, 2033-2048.
Menz, A., Weitbrecht, T., Gorbokon, N., Buscheck, F., Luebke, A. M., Kluth, M., Hube-Magg, C., Hinsch, A., Höflmayer, D., Weidemann, S., et al. (2021). Diagnostic and prognostic impact of cytokeratin 18 expression in human tumors: a tissue microarray study on 11,952 tumors. Molecular Medicine 27, 16.
Mi, H., and Thomas, P. (2009). PANTHER Pathway: an ontology-based pathway database coupled with data analysis tools. Methods Mol Biol 563, 123-140.
Mincheva-Nilsson, L., Baranov, V., Nagaeva, O., and Dehlin, E. (2016). Isolation and Characterization of Exosomes from Cultures of Tissue Explants and Cell Lines. Current Protocols in Immunology 115, 14.42.1-14.42.21.
Mizushima, N., and Yoshimori, T. (2007). How to Interpret LC3 Immunoblotting. Autophagy 3, 542-545.
Morgan, N. E., Cutrona, M. B., and Simpson, J. C. (2019). Multitasking Rab Proteins in Autophagy and Membrane Trafficking: A Focus on Rab33b. Int J Mol Sci 20.
Muller, L., Mitsuhashi, M., Simms, P., Gooding, W. E., and Whiteside, T. L. (2016). Tumor-derived exosomes regulate expression of immune function-related genes in human T cell subsets. Sci Rep 6, 20254.
Narayanan, A., Iordanskiy, S., Das, R., Van Duyne, R., Santos, S., Jaworski, E., Guendel, I., Sampey, G., Dalby, E., Iglesias-Ussel, M., et al. (2013). Exosomes Derived from HIV-1-infected Cells Contain Trans-activation Response Element RNA. J Biol Chem 288, 20014-20033.
New, J., and Thomas, S. M. (2019). Autophagy-dependent secretion: mechanism, factors secreted, and disease implications. Autophagy 15, 1682-1693.
Onishi, M., Yamano, K., Sato, M., Matsuda, N., and Okamoto, K. (2021). Molecular mechanisms and physiological functions of mitophagy. EMBO J 40.
Othman, N., Jamal, R., and Abu, N. (2019). Cancer-Derived Exosomes as Effectors of Key Inflammation-Related Players. Front Immunol 10.
Patel, K. K., Miyoshi, H., Beatty, W. L., Head, R. D., Malvin, N. P., Cadwell, K., Guan, J.-L., Saitoh, T., Akira, S., Seglen, P. O., et al. (2013). Autophagy proteins control goblet cell function by potentiating reactive oxygen species production. EMBO J 32, 3130-3144.
Pinto, D. O., DeMarino, C., Pleet, M. L., Cowen, M., Branscome, H., Al Sharif, S., Jones, J., Dutartre, H., Lepene, B., Liotta, L. A., et al. (2019). HTLV-1 Extracellular Vesicles Promote Cell-to-Cell Contact. Front Microbiol 10, 2147.
Pinto, D. O., Al Sharif, S., Mensah, G., Cowen, M., Khatkar, P., Erickson, J., Branscome, H., Lattanze, T., DeMarino, C., Alem, F., et al. (2021). Extracellular vesicles from HTLV-1 infected cells modulate target cells and viral spread. Retrovirology 18, 6.
Pircher, A., Hilbe, W., Heidegger, I., Drevs, J., Tichelli, A., and Medinger, M. (2011). Biomarkers in Tumor Angiogenesis and Anti-Angiogenic Therapy. Int J Mol Sci 12, 7077-7099.
Pleet, M. L., Mathiesen, A., DeMarino, C., Akpamagbo, Y. A., Barclay, R. A., Schwab, A., Iordanskiy, S., Sampey, G. C., Lepene, B., Ilinykh, P. A., et al. (2016). Ebola VP40 in Exosomes Can Cause Immune Cell Dysfunction. Front Microbiol 7.
Pleet, M. L., Branscome, H., DeMarino, C., Pinto, D. O., Zadeh, M. A., Rodriguez, M., Sariyer, I. K., El-Hage, N., and Kashanchi, F. (2018). Autophagy, EVs, and Infections: A Perfect Question for a Perfect Time. Front Cell Infect Microbiol 8, 362.
Poggio, M., Hu, T., Pai, C.-C., Chu, B., Belair, C. D., Chang, A., Montabana, E., Lang, U. E., Fu, Q., Fong, L., et al. (2019). Suppression of Exosomal PD-L1 Induces Systemic Anti-tumor Immunity and Memory. Cell 177, 414-427.e13.
Popova, T. G., Teunis, A., Magni, R., Luchini, A., Espina, V., Liotta, L. A., and Popov, S. G. (2015). Chemokine-Releasing Nanoparticles for Manipulation of Lymph Node Microenvironment. Nanomaterials (Basel) 5, 298-320.
Pulaski, B. A., and Ostrand-Rosenberg, S. (2000). Mouse 4T1 Breast Tumor Model. Current Protocols in Immunology 39, 20.2.1-20.2.16.
Raposo, G., and Stoorvogel, W. (2013). Extracellular vesicles: Exosomes, microvesicles, and friends. J Cell Biol 200, 373-383.
Rasool, S., Veyron, S., Soya, N., Eldeeb, M. A., Lukacs, G. L., Fon, E. A., and Trempe, J.-F. (2022). Mechanism of PINK1 activation by autophosphorylation and insights into assembly on the TOM complex. Molecular Cell 82, 44-59.e6.
Roberts, R. F., Tang, M. Y., Fon, E. A., and Durcan, T. M. (2016). Defending the mitochondria: The pathways of mitophagy and mitochondrial-derived vesicles. The International Journal of Biochemistry & Cell Biology 79, 427-436.
Sampey, G. C., Saifuddin, M., Schwab, A., Barclay, R., Punya, S., Chung, M.-C., Hakami, R. M., Asad Zadeh, M., Lepene, B., Klase, Z. A., et al. (2016). Exosomes from HIV-1-infected Cells Stimulate Production of Pro-inflammatory Cytokines through Trans-activating Response (TAR) RNA. J Biol Chem 291, 1251-1266.
Sanchez-Wandelmer, J., and Reggiori, F. (2013). Amphisomes: out of the autophagosome shadow?EMBO J 32, 3116-3118.
Shimoda, M., and Khokha, R. (2017). Metalloproteinases in extracellular vesicles. Biochimica et Biophysica Acta (BBA)—Molecular Cell Research 1864, 1989-2000.
Song, Y., Wu, L., and Yang, C. Exosomal PD-L1: an effective liquid biopsy target to predict immunotherapy response. Natl Sci Rev.
Stacker, S. A., Williams, S. P., Karnezis, T., Shayan, R., Fox, S. B., and Achen, M. G. (2014). Lymphangiogenesis and lymphatic vessel remodelling in cancer. Nature Reviews Cancer 14, 159-172.
Stephens, O. R., Grant, D., Frimel, M., Wanner, N., Yin, M., Willard, B., Erzurum, S. C., and Asosingh, K. (2020). Characterization and origins of cell-free mitochondria in healthy murine and human blood. Mitochondrion 54, 102-112.
Sun, A., Wei, J., Childress, C., Shaw, J. H., Peng, K., Shao, G., Yang, W., and Lin, Q. (2017). The E3 ubiquitin ligase NEDD4 is an LC3-interactive protein and regulates autophagy. Autophagy 13, 522-537.
Tamburro, D., Fredolini, C., Espina, V., Douglas, T. A., Ranganathan, A., Ilag, L., Zhou, W., Russo, P., Espina, B. H., Muto, G., et al. (2011). Multifunctional core-shell nanoparticles: discovery of previously invisible biomarkers. J. Am. Chem. Soc. 133, 19178-19188.
Tang, D., Kang, R., Zeh, H. J., and Lotze, M. T. (2010). High-mobility Group Box 1 [HMGB1] and Cancer. Biochim Biophys Acta 1799, 131.
Teng, Y., Ren, Y., Hu, X., Mu, J., Samykutty, A., Zhuang, X., Deng, Z., Kumar, A., Zhang, L., Merchant, M. L., et al. (2017). MVP-mediated exosomal sorting of miR-193a promotes colon cancer progression. Nature Communications 8, 14448.
Teunis, A. L., Popova, T. G., Espina, V., Liotta, L. A., and Popov, S. G. (2017). Immune-modulating Activity of Hydrogel Microparticles Contributes to the Host Defense in a Murine Model of Cutaneous Anthrax. Front Mol Biosci 4, 62.
Thomas, S. N., Vokali, E., Lund, A. W., Hubbell, J. A., and Swartz, M. A. (2014). Targeting the tumor-draining lymph node with adjuvanted nanoparticles reshapes the anti-tumor immune response. Biomaterials 35, 814-824.
Vader, P., Breakefield, X. O., and Wood, M. J. A. (2014). Extracellular vesicles: Emerging targets for cancer therapy. Trends Mol Med 20, 385-393.
Vella, L. J., Scicluna, B. J., Cheng, L., Bawden, E. G., Masters, C. L., Ang, C.-S., Willamson, N., McLean, C., Barnham, K. J., and Hill, A. F. (2017). A rigorous method to enrich for exosomes from brain tissue. J Extracell Vesicles 6.
Villa, E., Proics, E., Rubio-Patiño, C., Obba, S., Zunino, B., Bossowski, J. P., Rozier, R. M., Chiche, J., Mondragón, L., Riley, J. S., et al. (2017). Parkin-Independent Mitophagy Controls Chemotherapeutic Response in Cancer Cells. Cell Reports 20, 2846-2859.
Villa, E., Marchetti, S., and Ricci, J.-E. (2018). No Parkin Zone: Mitophagy without Parkin. Trends in Cell Biology 28, 882-895.
Vlassov, A. V., Magdaleno, S., Setterquist, R., and Conrad, R. (2012). Exosomes: current knowledge of their composition, biological functions, and diagnostic and therapeutic potentials. Biochim. Biophys. Acta 1820, 940-948.
Wang, J., Davis, S., Zhu, M., Miller, E. A., and Ferro-Novick, S. (2017). Autophagosome formation: Where the secretory and autophagy pathways meet. Autophagy 13, 973-974.
Xi, L., Peng, M., Liu, S., Liu, Y., Wan, X., Hou, Y., Qin, Y., Yang, L., Chen, S., Zeng, H., et al. (2021). Hypoxia-stimulated ATM activation regulates autophagy-associated exosome release from cancer-associated fibroblasts to promote cancer cell invasion. Journal of Extracellular Vesicles 10.
Yadav, B. S., Chanana, P., and Jhamb, S. (2015). Biomarkers in triple negative breast cancer: A review. World J Clin Oncol 6, 252-263.
Yoon, Y., Krueger, E. W., Oswald, B. J., and McNiven, M. A. (2003). The Mitochondrial Protein hFis1 Regulates Mitochondrial Fission in Mammalian Cells through an Interaction with the Dynamin-Like Protein DLP1. Mol Cell Biol 23, 5409-5420.
Yoshii, S. R., and Mizushima, N. (2017). Monitoring and Measuring Autophagy. Int J Mol Sci 18. (2019). Using Confidence Intervals to Compare Means.

1. A method comprising: selecting a living tissue of a human or an animal subject, applying a negative pressure on the living tissue; and harvesting extracellular vesicles (EVs) from interstitial fluid (IF) of the tissue, wherein the EVs are in a size in a range of about 30 nm-5000 nm, wherein the method is configured to maintain integrity of the living tissue for subsequent histopathological examination.

2. The method of claim 1, further comprises piercing the living tissue using a needle to collect the IF from the living tissue, wherein the needle comprises a hollow needle that applies a negative pressure in a range between 10 mmHg to about 100 mmHg.

3-4. (canceled)

5. The method of claim 2, wherein a portion of periphery of the needle comprises one or more holes having a dimension less than a cell and configured to allow entry of EVs present in a space inside the needle.

6-10. (canceled)

11. The method of claim 2, wherein a portion of the needle comprises one or more prongs, wherein the one or more prongs is configured to cover one or more holes, wherein the needle is configured to move relatively to one or more prongs to expose or close the one or more holes.

12. (canceled)

13. The method of claim 1, wherein the IF from the living tissue is collected in a device comprising a first chamber, wherein the first chamber comprises a matrix having (a) zeta potential between about −60 mV to about −20 mV, (b) a surface contact wetting angle less than 50 degrees and (c) a pore size in a range of 5 nm and less than 5000 nm.

14. The method of claim 13, further comprising centrifuging the device to allow passage of macromolecules comprising proteins from the first chamber to a second chamber of the device while retaining the EVs in the first chamber.

15. The method of claim 13, wherein a needle is connected to a syringe, which is further connected to the device.

16. (canceled)

17. The method of claim 1, wherein one or more biomarkers measured on the EVs determine (a) spatial origin of the EVs in the living tissue, (b) an information about a state of the living tissue, and/or (c) indicator of a cellular metabolic state of the living tissue.

18. The method of claim 17, wherein the living tissue comprises an ex-vivo tissue.

19. The method of claim 18, further comprising forming a section of the ex-vivo tissue, harvesting EVs on a solid phase comprising an affinity capture surface, and labelling the EVs.

20. (canceled)

21. The method of claim 18, wherein the ex-vivo tissue is placed on a matrix comprising a negatively charged net material and subjected to a negative pressure to collect the IF in a chamber.

22. (canceled)

23. The method of claim 16, wherein one or more biomarkers comprises CD9, CD63 and/or CD81.

24-25. (canceled)

26. The method of claim 1, wherein the method is configured not to physically rupture cells present within the living tissue.

27-36. (canceled)

37. The method of claim 17, wherein the EVs comprise CD81 and PINK1.

38. (canceled)

41. A composition comprising extracellular vesicles (EVs) harvested from a tissue, comprising proteins comprising mitophagy associated mitochondrial proteins produced during a cellular mitophagy of one or more cells of the tissue, wherein the tissue is subjected to a stress, wherein the EVs are configured to provide an indicator on a cellular metabolic state of the tissue.

42. The method of claim 1744, wherein the stress on the living tissue comprises an oxidative stress or a nutritive stress.

43-45. (canceled)

46. The method of claim 1744, wherein the EVs comprises a mitophagy associated mitochondrial proteins comprising at least one of PTEN-induced kinase 1 (PINK1), dynamin-related protein 1 (DRP1), mitochondrial fission 1 protein (FIS1), Ariadne RBR E3 Ubiquitin Protein Ligase 1 (ARIH1), HUWE1, HECT, UBA, WWE Domain Containing E3 Ubiquitin Protein Ligase 1, Smad ubiquitin regulatory factor 1 (SMURF1), p62, sequestome 1, LC3, Microtubule-associated protein 1A/1B-light chain 3, phosphatidylethanolamine (PE), Sec22B, SEC22 Homolog B, Vesicle Trafficking Protein, Ras-Related Protein Rab-33B (Rab33B), HSP90/CDC37, 90 kDa heat shock protein, Cell Division Cycle 37, Voltage-dependent anion-selective channel 1 (VDAC), Translocase of Outer Mitochondrial Membrane 34 (TOM34) or Vacuolar protein sorting ortholog 35 (VPS-35).

47-53. (canceled)

54. The method of claim 17, wherein one or more biomarkers present on the EVs determine cellular or spatial origin of the EVs.

55-56. (canceled)

57. The method of claim 17, wherein the living tissue is subjected to a therapy comprising a radiation therapy, immunotherapy, molecular targeted therapy, and/or a chemotherapy prior to biopsy of the tissue.

58. A method of treating, comprising: taking a composition comprising extracellular vesicles (EVs) comprising proteins comprising mitophagy associated mitochondrial proteins, wherein the proteins are modified by exposure of the cell under a stress, and administrating the composition in a lymph node of an animal or a human subject, wherein the method is configured to treat a disease.

59-70. (canceled)
